(19) United States
(12) Patent Application Publication
(10) Pub. No.: US 2026/0258471 A1
(43) Pub. Date: Sep. 3, 2026

(54) SYSTEMS AND METHODS FOR MICROCOLONY GROWTH AND MICROBIAL CELL CHARACTERIZATION

(71) Applicants: SAMAD TALEBPOUR, RICHMOND HILL (CA); ROBERT MAASKANT, KING CITY (CA); AYE AYE KHINE, THORNHILL (CA); STEPHEN WESLEY LEONARD, MARKHAM (CA); VILCY PARMAR, NORTH YORK (CA); ANNA KHIMCHENKO, THORNHILL (CA); MARYAM ASADISHEKARI, NORTH YORK (CA); SUKHDEV MANKU, TORONTO (CA); SANJESH YASOTHARAN, TORONTO (CA); TINO ALAVIE, THORNHILL (CA); ALALEH SAMIEI, RICHMOND HILL (CA)

(72) Inventors: SAMAD TALEBPOUR, RICHMOND HILL (CA); ROBERT MAASKANT, KING CITY (CA); AYE AYE KHINE, THORNHILL (CA); STEPHEN WESLEY LEONARD, MARKHAM (CA); VILCY PARMAR, NORTH YORK (CA); ANNA KHIMCHENKO, THORNHILL (CA); MARYAM ASADISHEKARI, NORTH YORK (CA); SUKHDEV MANKU, TORONTO (CA); SANJESH YASOTHARAN, TORONTO (CA); TINO ALAVIE, THORNHILL (CA); ALALEH SAMIEI, RICHMOND HILL (CA)

(21) Appl. No.: 19/289,247

(22) Filed: Aug. 4, 2025

Related U.S. Application Data
(63) Continuation of application No. 17/416,725, filed on Dec. 20, 2019, now abandoned.
(60) Provisional application No. 62/784,234, filed on Dec. 21, 2018.
(60) Provisional application No. 62/928,935, filed on Oct. 31, 2019.

(51) Int. Cl. C12Q 1/06; C12Q 1/18

(52) U.S. Cl. CPC C12Q 1/06; C12Q 1/18

(57) ABSTRACT

An integrated fluidic device is employed to perform microbial cell separation, in situ microcolony growth, and optional identification and antimicrobial susceptibility testing. While the integrated fluidic device is maintained in a closed state, microbial cell separation is performed to provide a microbial cell suspension that is contacted with a solid phase growth medium. A liquid component of the suspension is removed, thereby retaining microbial cells on the growth medium for incubation, growth, and subsequent harvesting and characterization. In some embodiments, antimicrobial susceptibility testing is performed by contacting growth media with a solid support having an antimicrobial agent provided thereon, such that the antimicrobial agent diffuses into a subregion of the growth medium that is accessible through an aperture surrounded, at least in part, by the solid support. Microbial cells retained on the surface of the subregion may be assessed for growth or inhibition in the presence of the antimicrobial agent.

### CROSS-REFERENCE TO RELATED APPLICATION

This application claims priority to U.S. Provisional Patent Application No. 62/784,234, titled “SYSTEMS AND METHODS FOR PERFORMING AND MONITORING RAPID MICROBIAL COLONY GROWTH” and filed on Dec. 21, 2018, the entire contents of which is incorporated herein by reference, and to U.S. Provisional Patent Application No. 62/928,935, titled “SYSTEMS AND METHODS FOR MICROCOLONY GROWTH AND MICROBIAL CELL CHARACTERIZATION” and filed on Oct. 31, 2019, the entire contents of which is incorporated herein by reference.

### BACKGROUND

The present disclosure relates to the growth, detection and characterization of microbial cells. More particularly, the present disclosure relates to microcolony growth and characterization and antimicrobial susceptibility testing.

Identifying causative organisms of microbial infection and determining their antimicrobial susceptibility profile is the main goal of diagnostic routing in clinical microbiology laboratories. As a common practice, this task is currently performed by drawing patient blood into culture bottles containing antibiotic absorbing agents, incubating the bottle in an environment that promotes growth of the blood microbial cell content, performing Gram stain to classify bacterial cells in terms of cell wall characteristic and morphology, sub-culturing the cells on solid phase growth media such as agar plates to obtain pure microbial colonies, partially or fully identifying the microbial cells, suspending the colony content in a media in a manner by which the cell concentration falls in a desired range, incubating aliquots of the cell-suspension in contact with different doses of selected antimicrobials in appropriate medium, and determining the minimum inhibitory concentration (MIC) from the growth profiles of the cell aliquots. The major shortcomings of this diagnostic routing are long time to result (of order of few days) and the possibility of preferential growth in the case of polymicrobial samples.

### SUMMARY

An integrated fluidic device is employed to perform microbial cell separation, in situ microcolony growth, and optional identification and antimicrobial susceptibility testing. While the integrated fluidic device is maintained in a closed state, microbial cell separation is performed to provide a microbial cell suspension that is contacted with a solid phase growth medium. A liquid component of the suspension is removed, thereby retaining microbial cells on the growth medium for incubation, growth, and subsequent harvesting and characterization. In some embodiments, antimicrobial susceptibility testing is performed by contacting growth media with a solid support having an antimicrobial agent provided thereon, such that the antimicrobial agent diffuses into a subregion of the growth medium that is accessible through an aperture surrounded, at least in part, by the solid support. Microbial cells retained on the surface of the subregion may be assessed for growth or inhibition in the presence of the antimicrobial agent.

Accordingly, in a first aspect, there is provided a method of processing a sample containing microbial cells, the method comprising:
introducing the sample into an integrated fluidic device, the integrated fluidic device comprising a sample processing module and a growth module;
while maintaining the integrated fluidic device in a closed state to prevent ingress of external microbial cells:
   processing the sample within the sample processing module to separate the microbial cells from the sample and obtain a microbial cell suspension, the microbial cell suspension comprising the microbial cells suspended within a liquid;
   transporting the microbial cell suspension from the sample processing module to the growth module such that the microbial cell suspension contacts a solid phase growth medium residing within the growth module, the solid phase growth medium being configured to promote microbial cell growth;
   removing at least a portion of the liquid from the microbial cell suspension such that at least one microbial cell is retained on a surface of the solid phase growth medium; and
   incubating at least the growth module under conditions suitable for promoting colony growth.

In some implementations of the method, at least a portion of the liquid is removed by absorption of the at least a portion of the liquid by the solid phase growth medium.

In some implementations of the method, the liquid is a first liquid and the solid phase growth medium is a gel-based medium, the method further comprising, prior to contacting the microbial cell suspension with the solid phase growth medium, subjecting the integrated fluidic device to a centrifugal force to remove a second liquid from the solid phase growth medium, thereby obtaining a partially dehydrated solid phase growth medium, such that when the microbial cell suspension is contacted with the partially dehydrated solid phase growth medium, the at least a portion of the first liquid is removed via absorption by the partially dehydrated solid phase growth medium. The centrifugal force may range between 1,000 g and 10,000 g. The sample may be processed within the sample processing module to separate the microbial cells from the sample according to a centrifugal-based separation method, and wherein the centrifugal force is applied to the solid phase growth medium during the centrifugal-based separation method. The centrifugal force may be applied in a direction that is less than 30 degrees from a surface normal associated with the surface of the solid phase growth medium. The centrifugal force may be applied in a direction that is perpendicular to the surface of the solid phase growth medium.

In some implementations of the method, the surface contacting the microbial cell suspension is a first surface, the solid phase growth medium further comprises a second surface opposing the first surface, the centrifugal force being applied such that the second liquid is removed from a region proximal to the second surface.

In some implementations of the method, the surface contacting the microbial cell suspension is a first surface, the solid phase growth medium further comprises a second surface opposing the first surface, the centrifugal force being applied such that a first region of the solid phase growth medium that is proximal to the first surface is more dehydrated than a second region of the solid phase growth medium that is proximal to the second surface.

In some implementations of the method, the second liquid is absorbed by an absorbent material in flow communication with the solid phase growth medium.

In some implementations of the method, a porous membrane resides between the solid phase growth medium and the absorbent material.

In some implementations of the method, the centrifugal force is a first centrifugal force and the method further comprises, after having contacted the partially dehydrated solid phase growth medium with the microbial cell suspension, subjecting the integrated fluidic device to a second centrifugal force to promote absorption of the at least a portion of the liquid from the microbial cell suspension by the partially dehydrated solid phase growth medium and retention of the at least one microbial cell on the surface. The second centrifugal force may range between 500 g and 4000 g.

In some implementations of the method, the solid phase growth medium is configured to passively absorb the at least a portion of the liquid. The solid phase growth medium may comprise a porous network and resides in at least a partially dehydrated state prior to contact with the microbial cell suspension. The solid phase growth medium may be provided as a partially dehydrated hydrogel.

In some implementations of the method, at least a portion of the liquid is evaporatively removed through a gas-permeable membrane.

In some implementations of the method, at least a portion of the liquid is evaporatively removed via active circulation of air.

In some implementations of the method, the microbial cells are separated via a separation method selected from the group consisting of filtration, immunomagnetic separation and microfluidic separation.

In some implementations of the method, at least one microbial cell retained on the surface of the solid phase growth medium is a Streptococcus pneumoniae microbial cell and wherein colony growth associated with the Streptococcus pneumoniae microbial cell is achieved in an absence of control of a carbon dioxide environment within the growth module.

In some implementations of the method, the sample is a whole blood sample and the microbial cells are separated from the sample in the sample processing module by: (i) mixing the whole blood sample and a blood lysis reagent, the blood lysis reagent comprising saponin, sodium polyanethole sulfonate and an alkaline buffer, to obtain a mixture having a concentration of saponin between 0.75 and 60 mg/ml, a concentration of sodium polyanethole sulfonate between 0.35 and 50 mg/ml and a pH between 7.8 and 10; and (ii) separating microbial cells from the mixture.

In some implementations, the method further comprises: (i) detecting a presence of a colony on the solid phase growth medium, the colony having a diameter of less than 100 microns; and (ii) harvesting microbial cells from the colony. In some implementations of the method, detecting the presence of the colony on the solid phase growth medium comprises: (i) obtaining a first image of the solid phase growth medium; (ii) obtaining a second image of the solid phase growth medium, wherein the second image is obtained after a time delay during incubation of the growth module; (iii) registering the first image to the second image using surface artefacts present in the image; (iv) performing image subtraction on the registered first and second images to remove surface artefacts from the second image, thereby obtaining a subtracted image; and (v) processing the subtracted image to identify a location of the colony. At least a subset of the surface artefacts may be inhomogeneities in the surface of the solid phase growth medium, and/or at least a subset of the surface artefacts may be lysis debris particles residing on the surface of the solid phase growth medium, the lysis debris particles having been generated by lysis of the sample with the sample processing module, where the lysis debris particles may be blood lysis debris particles, and where a mean particle diameter of the blood lysis debris particles may be less than 10 microns. The sample processing module may be configured such that an areal fraction of coverage of the solid phase growth medium by the lysis debris particles is less than 20 percent, 50 percent or 90 percent.

In some implementations, the method further comprises employing the harvested microbial cells to perform antimicrobial susceptibility testing. Prior to harvesting the microbial cells from the colony, the method may include interrogating the colony, without compromising a viability of the colony, to classify the microbial cells as belonging to a microbial cell class selected from a set of microbial classes.

The selected microbial cell class may be determined, at least in part, based on a measured growth rate of the colony. The selected microbial cell class of the microbial cells may be selected from the set of microbial cell classes comprising bacterial cells and fungal cells. The selected microbial cell class of the microbial cells may be selected from the set of microbial cell classes consisting of bacterial cells and fungal cells. The selected microbial cell class of the microbial cells may be selected from the set of microbial cell classes comprising gram positive bacterial cells, gram negative bacterial cells, and fungal cells. The selected microbial cell class of the microbial cells may be selected from the set of microbial cell classes consisting of gram positive bacterial cells, gram negative bacterial cells, and fungal cells.

The antimicrobial susceptibility testing may be performed using one or more antibiotics, wherein the one or more antibiotics are selected according to the selected microbial cell class. In some implementations the method further comprises employing the selected microbial cell class to determine when the colony is expected to contain a sufficient quantity of microbial cells to perform antimicrobial susceptibility testing; wherein the harvested microbial cells are harvested after a determination is made that the colony contains a sufficient quantity of microbial cells. The determination that the colony contains the sufficient quantity of microbial cells may be made based on the selected microbial cell class and an optically detected colony size measure associated with a size of the colony. The determination of when the colony contains the sufficient quantity of microbial cells may be based on a pre-determined relationship between the selected microbial cell class and the colony size measure.

The determination of when the colony contains the sufficient quantity of microbial cells may be based on a pre-determined relationship between the selected microbial cell class and a growth time duration. The determination of when the colony contains the sufficient quantity of microbial cells may be based, at least in part, on a measured growth rate of the colony. The determination that the colony contains the sufficient quantity of microbial cells may be further made based on an optically detected colony size measure associated with a size of the colony.

The solid phase growth media may be a chromogenic growth media, and wherein the selected microbial cell class is determined based on a detected spectral feature of the colony, and the spectral feature may be detected via Raman microscopy, via Fourier transform infrared spectroscopy microscopy, or via fluorescence microscopy.

In some implementations of the method, interrogating the colony to determine the selected microbial cell class comprises: (i) directing an optical beam onto the colony; (ii) obtaining an image scattered light from the colony; and (iii) processing the image to determine the selected microbial cell class.

In some implementations of the method, microbial cells are harvested from the colony prior to the colony being detectable by the naked eye. In some implementations of the method, microbial cells are harvested from the colony when the colony has a diameter between 70 microns and 100 microns. In some implementations of the method, microbial cells are harvested from the colony prior to the colony reaching a diameter of 100 microns. In some implementations of the method, microbial cells are harvested from the colony prior to the colony reaching a diameter of 50 microns. In some implementations of the method, microbial cells are harvested from the colony prior to the colony reaching a diameter of 70 microns.

In some implementations of the method, the colony is a first colony, the microbial cells harvested from the first colony are first microbial cells, and the method further comprises: (i) detecting a presence of a second colony on the solid phase growth medium; and (ii) harvesting second microbial cells from the second colony. In some implementations of the method, the antimicrobial susceptibility testing is performed using microbial cells harvested from both the first colony and the second colony.

In some implementations, the method further comprises, prior to performing the antimicrobial susceptibility testing, interrogating the first colony and the second colony to determine a presence or absence of a phenotypic correspondence between the first colony and the second colony. The presence or absence of the phenotypic correspondence between the first colony and the second colony may be determined by comparing first optical signals detected from the first colony with second optical signals detected from the second colony. The presence or absence of the phenotypic correspondence between the first colony and the second colony may be determined by comparing a first optical image of the first colony with a second optical image of the second colony.

In some implementations of the method, the selected microbial cell class is a first selected microbial cell class associated with a first type of the first microbial cells within the first colony, and wherein the presence or absence of the phenotypic correspondence between the first colony and the second colony may be determined by: (i) interrogating the second colony, without compromising a viability of the second colony, to determine a second selected microbial cell class associated with a second type of the second microbial cells within the second colony, wherein the second selected microbial cell class is selected from the set of microbial cell classes; and (ii) determining whether or not the first microbial cell class is the same as the second microbial cell class.

In some implementations of the method, the first microbial cell class is associated with a first species of the first microbial cells of the first colony, and wherein the second microbial cell class is associated with a second species of the second microbial cells of the second colony, and wherein a presence of the phenotypic correspondence may be established when the first species is determined to be the same as the second species.

The antimicrobial susceptibility testing may be performed using microbial cells from both the first microbial cells and the second microbial cells after having determined the presence of the phenotypic correspondence between the first colony and the second colony.

The phenotypic correspondence may be determined to be absent between the first microbial cells and the second microbial cells, and antimicrobial susceptibility testing may be performed separately using the first microbial cells and the second microbial cells to determine separate antimicrobial susceptibility measures for the first microbial cells and the second microbial cells.

In some implementations of the method, the selected microbial cell class is a preliminary selected microbial cell class, and the preliminary selected microbial cell class is determined according to a first classification method, and wherein the set of microbial cell classes is a first set of microbial cell classes, the method may further comprise, after having determined the phenotypic correspondence between the first colony and the second colony: interrogating the second microbial cells harvested from the second colony to determine a supplementary microbial cell class associated with the type of the second microbial cells, wherein the supplementary microbial cell class is selected from a second set of microbial cell classes, wherein the supplementary microbial cell class is determined according to a second classification method. The second set of microbial cell classes may include a greater number of microbial cell classes than the first set of microbial cell classes. The supplementary microbial cell class may be absent from the first set of microbial cell classes. The supplementary microbial cell class may be a species-level microbial cell class. The first set of microbial cell classes may be absent of species-level microbial cell classes, and wherein the second set of microbial cell classes may comprise a plurality of species-level microbial cell classes. The second classification method may be capable of determining a given microbial cell class with greater confidence than the first classification method. The supplementary microbial cell class may be determined using matrix assisted laser desorption/ionization mass spectrometry, Raman detection and/or Fourier transform infrared spectroscopy.

In some implementations of the method, the second microbial cells from the second colony are harvested after harvesting the first microbial cells from the first colony, and wherein the second colony may be incubated for a longer time duration than the first colony, such that the second colony, when harvested, is larger than the first colony, when harvested.

In some implementations, the method further comprises determining when the second colony is expected to contain a sufficient quantity of microbial cells to facilitate the determination of the supplementary microbial cell class by the second classification method; wherein the second microbial cells are harvested from the second colony after a determination is made that the second colony contains the sufficient quantity of microbial cells. The determination that the second colony contains a sufficient number of microbial cells may be made after having initiated the antimicrobial susceptibility testing on the first microbial cells from the first colony, and wherein the determination of the supplementary microbial cell class associated with the second microbial cells is made prior to the completion of the antimicrobial susceptibility testing. The second colony may be incubated to facilitate further colony growth after the first microbial cells are harvested and before the second microbial cells are harvested.

In some implementations, the method further comprises reporting the supplementary microbial cell class associated with the second microbial cells and a minimum inhibitory concentration associated with the first microbial cells.

In some implementations of the method, the solid phase growth medium is a first solid phase growth medium and the microbial cell suspension is a first microbial suspension, and wherein the antimicrobial susceptibility testing is performed by: (i) resuspending the harvested microbial cells, thereby obtaining a second microbial cell suspension; (ii) dispensing the second microbial cell suspension onto additional solid phase growth media at a plurality of locations, each location having a different local antibiotic concentration; and (iii) monitoring the plurality of locations to infer an antimicrobial susceptibility of the microbial cells. The additional solid phase growth media may have a hydrophobic layer provided thereon and with plurality of apertures formed in the hydrophobic layer, wherein each aperture is formed over a respective location, and wherein the liquid is dispensed at each location through a respective aperture.

In some implementations of the method, the solid phase growth medium is a first solid phase growth medium and the microbial cell suspension is a first microbial suspension, and wherein the antimicrobial susceptibility testing may be performed by: (i) resuspending the harvested microbial cells, thereby obtaining a second microbial cell suspension; (ii) providing a solid support that at least partially surrounds an aperture, the solid support comprising a contact surface, wherein a chemical agent is provided on the contact surface and/or impregnated beneath the contact surface; (iii) contacting a second phase growth medium with the contact surface of the solid support such that a subregion of the second solid phase growth medium is accessible through the aperture, and such that at least a portion of the chemical agent diffuses inwardly into the subregion; (iv) depositing a volume of the second microbial cell suspension onto a surface of the subregion, such that microbial cells within the second microbial cell suspension are retained on the surface of the subregion; (v) incubating the second solid phase growth medium over a time duration that is sufficiently long to permit exposure of the retained microbial cells to the chemical agent; and (vi) detecting a presence or absence of microbial cell growth within the subregion.

In another aspect, there is provided a method of processing a sample suspected of containing microbial cells, the method comprising:
contacting a suspension of viable microbial cells with a solid phase growth medium under conditions suitable for promoting growth of the viable microbial cells;
detecting a presence of a colony on the solid phase growth medium, the colony having a diameter of less than 100 microns; optically interrogating the colony to identify a microbial cell class associated with the colony;
employing the microbial cell class to determine when the colony is expected to contain a sufficient quantity of microbial cells to perform antimicrobial susceptibility testing;
after the colony has grown to contain the sufficient quantity of microbial cells for antimicrobial susceptibility testing, harvesting microbial cells from the colony; and
employing the harvested microbial cells to perform antimicrobial susceptibility testing.

In some implementations of the method, the colony is a first colony, the microbial cells harvested from the first colony are first microbial cells, and the method further comprises: (i) detecting a presence of a second colony on the solid phase growth medium; and (ii) harvesting second microbial cells from the second colony.

The antimicrobial susceptibility testing may be performed using microbial cells harvested from both the first colony and the second colony.

In some implementations, the method further comprises, prior to performing the antimicrobial susceptibility testing, interrogating the first colony and the second colony to determine a presence or absence of a phenotypic correspondence between the first colony and the second colony. The presence or absence of the phenotypic correspondence between the first colony and the second colony may be determined by comparing first optical signals detected from the first colony with second optical signals detected from the second colony. The presence or absence of the phenotypic correspondence between the first colony and the second colony may be determined by comparing a first optical image of the first colony with a second optical image of the second colony.

In some implementations of the method, the selected microbial cell class is a first selected microbial cell class associated with a first type of the first microbial cells within the first colony, and wherein the presence or absence of the phenotypic correspondence between the first colony and the second colony may be determined by: (i) interrogating the second colony, without compromising a viability of the second colony, to determine a second selected microbial cell class associated with a second type of the second microbial cells within the second colony, wherein the second selected microbial cell class is selected from the set of microbial cell classes; and (ii) determining whether or not the first microbial cell class is the same as the second microbial cell class. The first microbial cell class may be associated with a first species of the first microbial cells of the first colony, and wherein the second microbial cell class is associated with a second species of the second microbial cells of the second colony, and wherein a presence of the phenotypic correspondence may be established when the first species is determined to be the same as the second species.

In some implementations of the method, the antimicrobial susceptibility testing is performed using microbial cells from both the first microbial cells and the second microbial cells after having determined the phenotypic correspondence between the first colony and the second colony.

In some implementations of the method, the phenotypic correspondence is determined to be absent between the first microbial cells and the second microbial cells, and antimicrobial susceptibility testing is performed separately using the first microbial cells and the second microbial cells to determine separate antimicrobial susceptibility measures for the first microbial cells and the second microbial cells.

In some implementations of the method, the selected microbial cell class is a preliminary selected microbial cell class, and wherein the preliminary selected microbial cell class is determined according to a first classification method, and wherein the set of microbial cell classes is a first set of microbial cell classes, the method further comprising, after having determined the correspondence between the first colony and the second colony: interrogating the second microbial cells harvested from the second colony to determine a supplementary microbial cell class associated with the type of the second microbial cells, wherein the supplementary microbial cell class is selected from a second set of microbial cell classes, wherein the supplementary microbial cell class is determined according to a second classification method. The second set of microbial cell classes may include a greater number of microbial cell classes than the first set of microbial cell classes. The supplementary microbial cell class may be absent from the first set of microbial cell classes. The supplementary microbial cell class may be a species-level microbial cell class. The first set of microbial cell classes may be absent of species-level microbial cell classes, and wherein the second set of microbial cell classes comprises a plurality of species-level microbial cell classes. The second classification method may be capable of determining a given microbial cell class with greater confidence than the first classification method. The supplementary microbial cell class may be determined using matrix assisted laser desorption/ionization mass spectrometry. The supplementary microbial cell class may be determined using Raman detection and/or Fourier transform infrared spectroscopy.

In some implementations of the method, the second microbial cells from the second colony are harvested after harvesting the first microbial cells from the first colony, and wherein the second colony is incubated for a longer time duration than the first colony, such that the second colony, when harvested, is larger than the first colony, when harvested.

In some implementations, the method further comprises: determining when the second colony is expected to contain a sufficient quantity of microbial cells to facilitate the determination of the supplementary microbial cell class by the second classification method; wherein the second microbial cells are harvested from the second colony after a determination is made that the second colony contains the sufficient quantity of microbial cells. The determination that the second colony contains a sufficient number of microbial cells may be made after having initiated the antimicrobial susceptibility testing on the first microbial cells from the first colony, and wherein the determination of the supplementary microbial cell class associated with the second microbial cells is made prior to the completion of the antimicrobial susceptibility testing. The second colony may be incubated to facilitate further colony growth after the first microbial cells are harvested and before the second microbial cells are harvested.

In some implementations, the method further comprises reporting the supplementary microbial cell class associated with the second microbial cells and a minimum inhibitory concentration associated with the first microbial cells.

In some implementations of the method, the suspension of viable microbial cells is obtained from a whole blood sample.

In another aspect, there is provided an integrated fluidic device for separating and growing viable microbial cells, the integrated fluidic device comprising:
a separation region configured to facilitate separation of microbial cells from a sample under suitable actuation of the integrated fluidic device; and
a colony growth region comprising a solid phase growth medium, wherein the colony growth region is configured to receive, under suitable actuation of the integrated fluidic device, separated microbial cells from an output of the separation region, such that the separated microbial cells are contacted with the solid phase growth medium, while maintaining an internal flow path of the integrated fluidic device in a closed state, thereby preventing ingress of external microbial cells.

In some implementations of the device, the colony growth region may be configured to facilitate monitoring of growth of the separated microbial cells residing on the solid phase growth medium during incubation under conditions suitable for promoting growth of the separated microbial cells. The solid phase growth medium may be configured to passively absorb a liquid in which the separated microbial cells are delivered from the separation region.

The solid phase growth medium may comprise a porous network and is provided is in a partially-hydrated state. The solid phase growth medium may be provided as a partially hydrated hydrogel.

In some implementations of the device, the colony growth region is detachably removable from a remainder of the integrated fluidic device.

In another aspect, there is provided a method of determining an effect of a chemical agent on growth of microbial cells, the method comprising:
providing a microbial cell suspension containing the microbial cells;
providing a solid support that at least partially surrounds an aperture, the solid support comprising a contact surface, wherein a chemical agent is provided on the contact surface and/or impregnated beneath the contact surface;
contacting a solid phase growth medium with the contact surface of the solid support such that a subregion of the solid phase growth medium is accessible through the aperture, and such that at least a portion of the chemical agent diffuses inwardly into the subregion;
depositing a volume of the microbial cell suspension onto a surface of the subregion, such that microbial cells within the microbial cell suspension are retained on the surface of the subregion;
incubating the solid phase growth medium over a time duration that is sufficiently long to permit exposure of the retained microbial cells to the chemical agent; and
detecting a presence or absence of microbial cell growth within the subregion.

In some implementations of the method, the contact surface may comprise a planar contact surface, and wherein the solid support is contacted with the solid phase growth medium such that the planar contact surface contacts a surface of the solid phase growth medium and at least partially surrounds the subregion, and such that a portion of the chemical agent diffuses from the planar contact surface into the subregion. The solid support may fully surround the aperture. The solid support may further comprise a flashing feature residing adjacent to the aperture, the flashing feature being configured such that when the planar contact surface is contacted with the solid phase growth medium, the flashing feature is submerged beneath the surface of the solid phase growth medium, thereby preventing or reducing ingress of the microbial cell suspension between the contact surface and the surface of the solid phase growth medium. The flashing feature may be configured to penetrate the solid phase growth medium to a depth of less than 250 microns. The flashing feature may be configured to penetrate the solid phase growth medium to a depth of less than 100 microns.

In some implementations of the method, at least a portion of the solid support may have an annular shape.

In some implementations of the method, the solid support may comprise a lateral confinement component located further from the aperture than the planar contact surface, the lateral confinement component being configured such that when the planar contact surface is contacted with the solid phase growth medium, the lateral confinement component is submerged within the solid phase growth medium. The lateral confinement component may fully surround the aperture.

In some implementations of the method, the contact surface may comprise a lateral contact surface located further from the aperture than the planar contact surface, the lateral contact surface being configured such that when the planar contact surface is contacted with the solid phase growth medium, the lateral contact surface is submerged within the solid phase growth medium with the lateral contact surface facing toward the subregion, such that chemical agent diffuses from both the planar contact surface and the lateral contact surface into the subregion. The lateral contact surface may fully surround the aperture. The lateral contact surface may be configured such that when the planar contact surface is contacted with the solid phase growth medium, the lateral contact surface is inserted into the solid phase growth medium to a depth exceeding 1 mm. The lateral contact surface may be configured such that when the planar contact surface is contacted with the solid phase growth medium, the lateral contact surface is inserted into the solid phase growth medium to a depth exceeding 2 mm.

In some implementations of the method, the solid support may comprise a tubular component, and wherein at least a distal surface region of an inner surface of the tubular component is coated with and/or impregnated with the chemical agent, and wherein the tubular component is contacted with the solid phase growth medium such that at least a portion of the distal surface region is submerged within the solid phase growth medium, and such that the chemical agent diffuses inwardly within the subregion of the solid phase growth medium that resides within a lumen of the tubular component. The tubular component may be inserted into the solid phase growth medium such that a proximal portion of the tubular component extends outwardly from the solid phase growth medium, and wherein the volume of the microbial cell suspension is dispensed into the proximal portion of the tubular component. The tubular component may be inserted such that a distal end of the tubular component contacts a support surface that supports the solid phase growth medium, thereby enclosing the subregion and confining diffusion of the chemical agent within the tubular component. The support surface may comprise one or more mating features provided therein or thereon, the one or more mating features being configured to contact the distal end of the tubular component. The one or more mating features may comprise one or both of a projection and a recess. The one or more mating features may fully surround the distal end of the tubular component. The tubular component may be a cylindrical component. A wall thickness of a distal portion of the tubular component may be less than 500 microns.

In some implementations of the method, the chemical agent is uniformly distributed on the contact surface.

In some implementations of the method, the chemical agent is provided at a plurality of separated regions on the contact surface.

In some implementations of the method, one or more of an area density and a subsurface density of the chemical agent spatially varies along the contact surface.

The chemical agent may be provided on the contact surface according to a gradient in one or more of the local area density and the subsurface density. The gradient may be provided such that the one or more of the local area density and the subsurface density of the chemical agent is lowest in a surface region that is closest to the aperture.

In some implementations of the method, the chemical agent may be provided on the contact surface with a suitable quantity and a suitable spatial distribution such that a concentration of the chemical agent immediately below a central portion of the surface of the subregion varies by less than 10% between one hour and three hours after contacting the contact surface with the solid phase growth medium.

In some implementations of the method, the chemical agent may be provided on the contact surface with a suitable quantity and a suitable spatial distribution such that a concentration of the chemical agent immediately below a central portion of the surface of the subregion varies by less than 5% between one hour and three hours after contacting the contact surface with the solid phase growth medium.

In some implementations of the method, the chemical agent may be provided on the contact surface with a suitable quantity and a suitable spatial distribution such that a concentration of the chemical agent immediately below a central portion of the surface of the subregion varies by less than 10% between two hours and four hours after contacting the contact surface with the solid phase growth medium.

In some implementations of the method, the chemical agent may be provided on the contact surface with a suitable quantity and a suitable spatial distribution such that a concentration of the chemical agent immediately below a central portion of the surface of the subregion may vary by less than 5% between two hours and four hours after contacting the contact surface with the solid phase growth medium.

In some implementations of the method, the solid phase growth medium may be contacted with the contact surface such that a concentration of the chemical agent immediately below a central portion of the surface of the subregion reaches a maximum concentration within 30 minutes of contact between the solid phase growth medium and the contact surface.

In some implementations of the method, the solid support may comprise a hydrophobic upper surface configured to facilitate retention of the volume of the microbial cell suspension over the subregion. The hydrophobic upper surface may be beveled toward the aperture to assist in retention of the volume of the microbial cell suspension over the subregion.

In some implementations of the method, a minimum width of the aperture may be less than 5 mm, less than 2 mm, or less than 1 mm.

In some implementations of the method, the number of microbial cells within the volume of the microbial cell suspension deposited onto the surface of the subregion may be less than 50, less than 20, or less than 10.

In some implementations of the method, the volume of the microbial cell suspension deposited onto the surface of the subregion may be less than 5 microliters or less than 2 microliters.

In some implementations of the method, the solid phase growth medium is retained within a microwell, and wherein a volume of the solid phase growth medium may be less than 100 microliters or less than 50 microliters.

In some implementations of the method, a thickness of the solid phase growth medium may be less than 2 mm or less than 1 mm.

In some implementations of the method, the chemical agent may be an antimicrobial agent.

In some implementations of the method, the microbial cell suspension may be obtained by processing a whole blood sample in an absence of blood culture.

In some implementations of the method, the microbial cell suspension may be obtained from a blood culture bottle in an absence of performing subculture. The microbial cell suspension may be obtained by diluting a blood culture sample.

In some implementations of the method, detecting the presence or absence of microbial cell growth within the subregion may be performed by obtaining one or more images of the surface of the subregion and processing the one or more image. In some implementations, the method further comprises: (i) providing one or more additional solid supports, each additional solid support at least partially surrounding a respective additional aperture, each additional solid support comprising a respective additional contact surface, wherein each additional contact surface has a different amount of the chemical agent provided thereon and/or impregnated therebeneath; (ii) contacting the solid phase growth medium with each additional contact surface such that additional subregions of the solid phase growth medium are accessible through the respective additional apertures, and such that at least a portion of the chemical agent diffuses inwardly into each respective additional subregions from each respective additional contact surface; (iii) depositing additional volumes of the microbial cell suspension onto a respective surface of each additional subregion, such that microbial cells within the microbial cell suspension are retained on the respective surfaces of the additional subregions; and (iv) after incubating the solid phase growth medium, detecting a presence or absence of microbial cell growth within each subregion.

In some implementations of the method, may further comprise determining a minimum inhibitory concentration of the chemical agent based on the assessment of the presence or absence of microbial cell growth within the subregions. The minimum inhibitory concentration may be determined according to an estimated concentration or concentration range of the chemical agent below the surface of each subregion during incubation of the solid phase growth medium. The solid support and the additional solid supports may be mechanically coupled and form an array of solid supports. The solid phase growth medium may be supported by a solid phase growth medium support structure, the support structure comprising a plurality of microwells, each microwell comprising a respective volume of the solid phase growth medium, and wherein the array of solid supports is contacted with the solid phase growth medium such that each contact surface of the array of solid supports is contacted with a different respective volume of the solid phase growth medium in a different respective microwell. The array of solid supports and the solid phase growth medium support structure may comprise a keyed feature that facilitates alignment between the respective contact surfaces and the respective microwells. The keyed feature may facilitate alignment of one or more of a lateral position and a depth of each contact surface relative to the respective microwells.

In another aspect, there is provided a method of determining an effect of a chemical agent on growth of microbial cells, the method comprising:
providing a microbial cell suspension containing the microbial cells;
contacting a solid phase growth medium with the chemical agent at one or more contact regions that at least partially surround and reside adjacent to a subregion of the solid phase growth medium, such that at least a portion of the chemical agent diffuses into the subregion from the one or more contact regions, wherein the one or more contact regions are provided such that a spatial extent of the subregion, when measured in at least one direction parallel to a surface of the solid phase growth medium, is less than 5 mm;
depositing a volume of the microbial cell suspension onto a surface of the subregion, such that microbial cells within the microbial cell suspension are retained on the surface of the subregion;
incubating the solid phase growth medium over a time duration that is sufficiently long to permit exposure of the retained microbial cells to the chemical agent; and
detecting a presence or absence of microbial cell growth within the subregion.

In another aspect, there is provided a method of introducing a chemical agent into a solid phase growth medium, the method comprising:
providing a solid support that at least partially surrounds an aperture, the solid support comprising a contact surface, wherein a chemical agent is provided on the contact surface and/or impregnated beneath the contact surface;
contacting the solid phase growth medium with the contact surface of the solid support such that a subregion of the solid phase growth medium is accessible through the aperture, and such that at least a portion of the chemical agent diffuses inwardly into the subregion.

In another aspect, there is provided a device for assessing an effect of a chemical agent on microbial cells, the device comprising:
a solid support at least partially surrounding an aperture, the solid support comprising a contact surface having the chemical agent provided thereon and/or impregnated thereunder, such that after contact of the contact surface of the solid support with a solid phase growth medium, the chemical agent diffuses inwardly, at least in part, from the contact surface into a subregion of the solid phase growth medium that is accessible through the aperture, thereby permitting exposure of microbial cells to the antimicrobial agent when a microbial cell suspension containing the microbial cells is inoculated onto the subregion.

In some implementations of the device, the contact surface may comprise a planar contact surface. The solid support may fully surround the aperture. The solid support may further comprise a flashing feature residing adjacent to the aperture, the flashing feature being configured such that when the planar contact surface is contacted with the solid phase growth medium, the flashing feature is submerged beneath the surface of the solid phase growth medium, thereby preventing or reducing ingress of the microbial cell suspension between the contact surface and the surface of the solid phase growth medium. The flashing feature may be configured to penetrate the solid phase growth medium to a depth of less than 250 microns when the planar contact surface contacts the surface of the solid phase growth medium. The flashing feature may be configured to penetrate the solid phase growth medium to a depth of less than 100 microns when the planar contact surface contacts the surface of the solid phase growth medium.

In some implementations of the device, at least a portion of the solid support has an annular shape.

In some implementations of the device, the solid support may comprise a lateral confinement component located further from the aperture than the planar contact surface, the lateral confinement component being configured such that when the planar contact surface is contacted with the solid phase growth medium, the lateral confinement component is submerged within the solid phase growth medium. The lateral confinement component may fully surround the aperture.

In some implementations of the device, the contact surface may comprise a lateral contact surface located further from the aperture than the planar contact surface, the lateral contact surface being configured such that when the planar contact surface is contacted with the solid phase growth medium, the lateral contact surface is submerged within the solid phase growth medium with the lateral contact surface facing toward the subregion, such that chemical agent diffuses from both the planar contact surface and the lateral contact surface into the subregion. The lateral contact surface may fully surround the aperture. The lateral contact surface may be configured such that when the planar contact surface is contacted with the solid phase growth medium, the lateral contact surface is inserted into the solid phase growth medium to a depth exceeding 1 mm. The lateral contact surface may be configured such that when the planar contact surface is contacted with the solid phase growth medium, the lateral contact surface is inserted into the solid phase growth medium to a depth exceeding 2 mm.

In some implementations of the device, the solid support comprises a tubular component, and wherein at least a distal surface region of an inner surface of the tubular component is coated with and/or impregnated with the chemical agent, such that when at least a portion of the distal surface region is submerged within the solid phase growth medium, the chemical agent diffuses inwardly within the subregion of the solid phase growth medium that resides within a lumen of the tubular component. The tubular component may be inserted into the solid phase growth medium such that a proximal portion of the tubular component extends outwardly from the solid phase growth medium, and wherein the volume of the microbial cell suspension is dispensed into the proximal portion of the tubular component. The tubular component may be inserted such that a distal end of the tubular component contacts a support surface that supports the solid phase growth medium, thereby enclosing the subregion and confining diffusion of the chemical agent within the tubular component. The tubular component may be a cylindrical component. A wall thickness of a distal portion of the tubular component may be less than 500 microns.

In some implementations of the device, the chemical agent may be uniformly distributed on the contact surface.

In some implementations of the device, the chemical agent may be provided at a plurality of separated regions on the contact surface.

In some implementations of the device, one or more of a local area density and the subsurface density of the chemical agent spatially varies along the contact surface. The chemical agent may be provided on the contact surface according to a gradient in one or more of the local area density and the subsurface density. The area density gradient may be provided such that the one or more of the local area density and the subsurface density of the chemical agent is lowest in a surface region that is closest to the aperture.

In some implementations of the device, the solid support may comprise a hydrophobic upper surface. The hydrophobic upper surface may be beveled toward the aperture to assist in retention of the volume of the microbial cell suspension over the subregion.

In some implementations of the device, a minimum width of the aperture may be less than 5 mm, less than 2 mm, or less than 1 mm.

In some implementations of the device, the chemical agent is an antimicrobial agent.

In some implementations, the device further comprises one or more additional solid supports, each additional solid support at least partially surrounding a respective additional aperture, each additional solid support comprising a respective additional contact surface, wherein each additional contact surface has a different amount of the chemical agent provided thereon and/or impregnated therebeneath.

The solid support and the additional solid supports may be mechanically coupled and form an array of solid supports.

In another aspect, there is provided a kit comprising: (i) a device as described above, further comprises one or more additional solid supports, each additional solid support at least partially surrounding a respective additional aperture, each additional solid support comprising a respective additional contact surface, wherein each additional contact surface has a different amount of the chemical agent provided thereon and/or impregnated therebeneath; and (ii) a solid phase growth medium support structure, the support structure comprising a plurality of microwells, each microwell comprising a respective volume of the solid phase growth medium, the solid phase growth medium support structure being configured to be contactable with the array of solid supports, each contact surface of the array of solid supports is contacted with a different respective volume of the solid phase growth medium in a different respective microwell. One or more of the array of solid supports and the solid phase growth medium support structure may comprise a keyed feature that facilitates alignment between the respective contact surfaces and the respective microwells. The keyed feature may facilitate alignment of one or more of a lateral position and a depth of each contact surface relative to the respective microwells.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 schematically illustrates two example functional modules of an example integrated fluidic cartridge intended for the separation of microbial cells from a sample and the subsequent seeding of the separated microbial cells onto a solid phase growth media in a closed cartridge configuration.

FIGS. 2A and 2B show top and side views, respectively, of an example growth module of an integrated sample processing and growth fluidic cartridge for receiving and seeding a microbial cell suspension for the subsequent growth of microcolonies.

FIG. 3A illustrates a section of a blood agar plate imaged by an upright reflected-illumination (epi) bright-field (BF) metallurgical microscope with 5× infinite plan objective. One μL of microbial cell suspension, obtained from whole blood which was treated by selectively lysing with a blood lysis reagent composed of saponin and sodium polyanethole sulfonate (SPS), followed by two centrifugal wash cycles, was dispensed on the plate and allowed to air dry before obtaining the microscopic image. The region over which the sample had spread is indicated by 312.

FIG. 3B illustrates a section of a blood agar plate imaged by an upright reflected-illumination (epi) bright-field (BF) metallurgical microscope with 5× infinite plan objective. One μL of microbial cell suspension, obtained from whole blood which was treated by selectively lysing with an alkaline blood lysis reagent including saponin, SPS, Triton-X100, and carbonate-bicarbonate buffer, followed by 2 wash cycles, was dispensed on the plate and allowed to air dry before taking the microscopic image.

FIG. 3C illustrates the blood debris size distribution obtained using the blood lysis reagent employed when processing the sample according to the method described with reference to FIG. 3B. One μL of microbial cell suspension, obtained from whole blood, which was treated by selectively lysing with an alkaline blood lysis reagent including saponin, SPS, Triton-X100, and carbonate-bicarbonate buffer followed by 2 or 4 wash cycles was dispensed on the plate and was allowed to air dry before taking microscopic image by 10× infinite plan objective. The image was analyzed for particle size distribution and the histogram of the particle size distributions was plotted for both 2 wash cycle (left) and 4 wash cycle (right).

FIGS. 4A-C schematically illustrate an example growth module that is optionally detachable from the integrated fluidic cartridge for separate incubation and monitoring after microbial cell seeding (under suitable environmental conditions for growth of microbial cell microcolonies).

FIGS. 5A-D schematically illustrate an example centrifugal method for contacting the microbial suspension of a sample on the gel-based solid phase growth medium within a growth chamber of the growth module. As the gel is centrifuged in shown in FIG. 5A and subjected to a centrifugal force, a portion of its liquid (e.g. water) component is forced outward (relative to the centrifugation axis) and such that after centrifugation, as shown in FIG. 5B, the gel surface is partially dewatered. In FIG. 5C, the microbial cell suspension is contacted with the gel surface and its liquid component is absorbed by the dewatered gel surface (e.g. optionally assisted gravitationally or via further centrifugation), thereby retaining the microbial cells on the gel surface, as shown in FIG. 5D.

FIG. 5E plots the fractional water loss of gels of various compositions after centrifugation. Each gel was placed on a membrane with pore size of 0.45 μm and centrifugated for 8 minutes at 3200 g.

FIG. 5F plots the fractional water loss of gels of various compositions after centrifugation. Each gel was placed on a membrane with pore size of 5 nm and centrifugated for 8 minutes at 3200 g.

FIG. 5G plots the dewatering (water loss) and rehydration (water gain) levels of gels with different compositions after centrifugation on a membrane with pore size of 5 nm and centrifugated for 8 minutes at 3200 g and followed by 20 minutes of soaking in water.

FIG. 5H plots the level of partial-dehydration of various gels through evaporation followed by rehydration via soaking in water.

FIG. 51 schematically illustrates one example embodiment for removing the centrifugally exuded liquid from the gel through an enforced membrane, during the steps shown in FIGS. 5A and 5B.

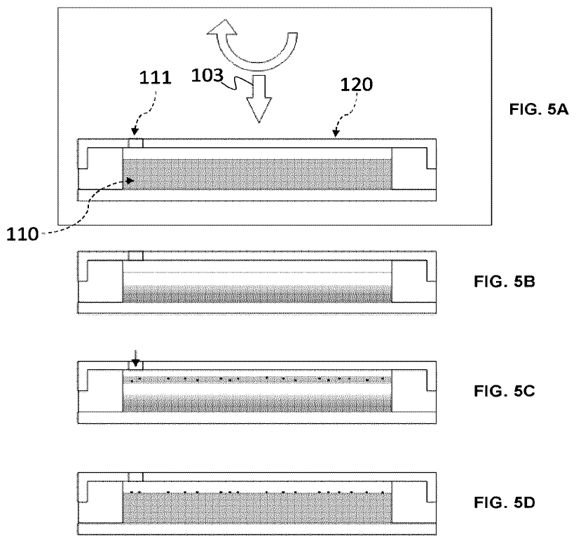

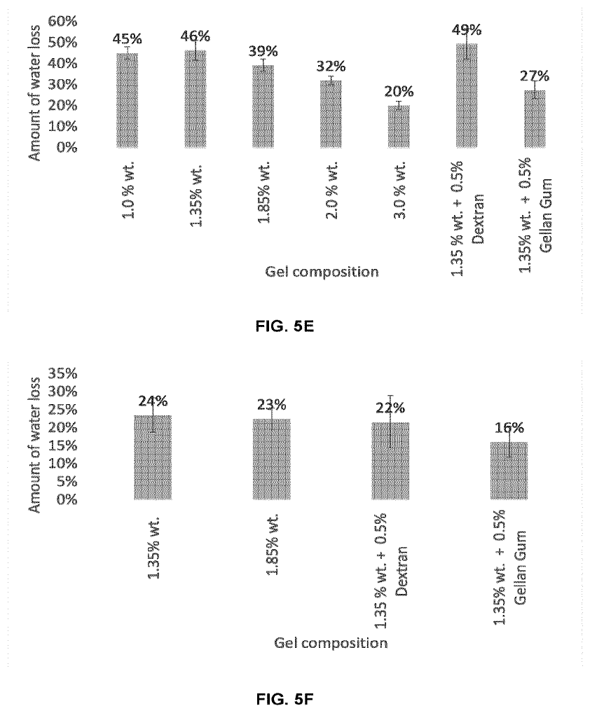

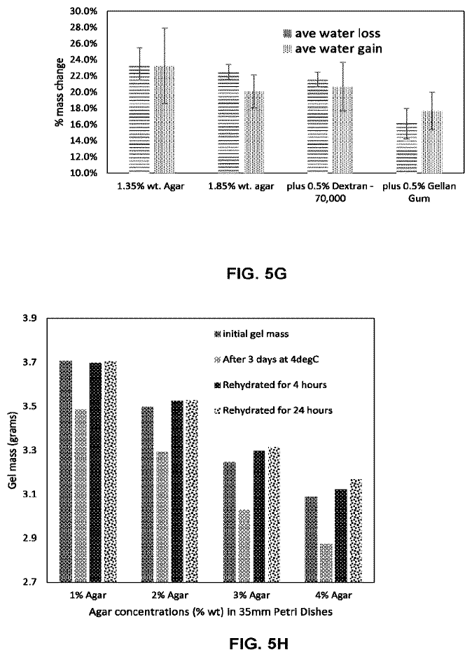

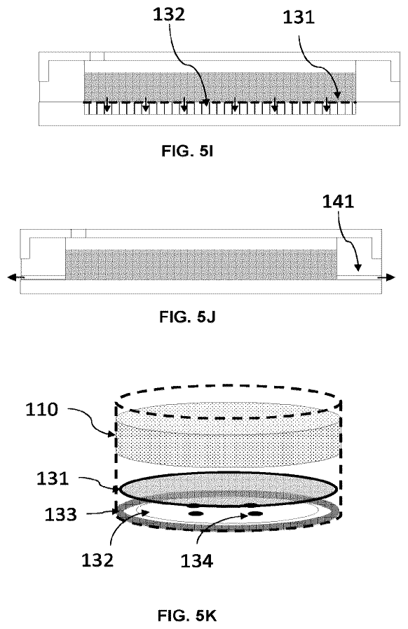

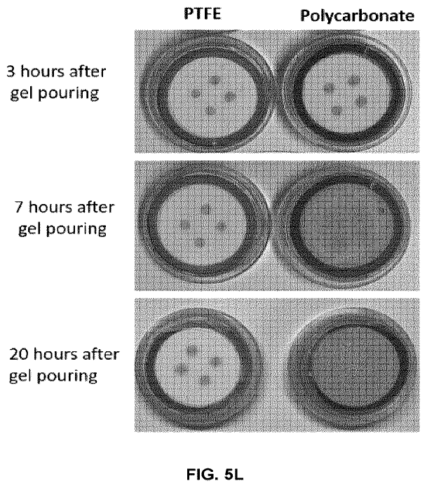

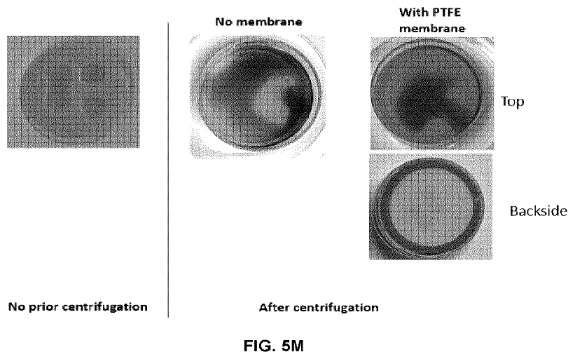

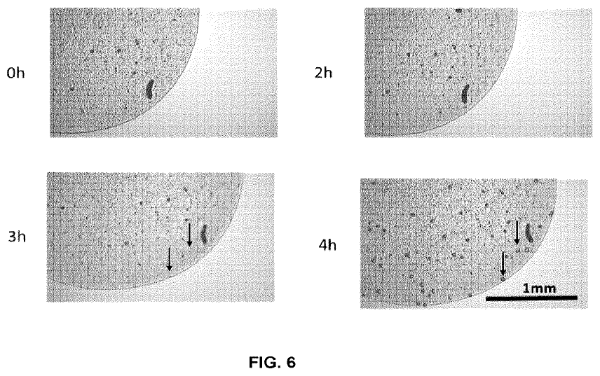

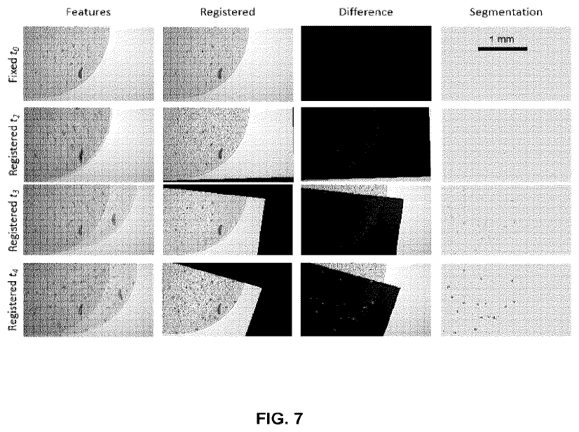

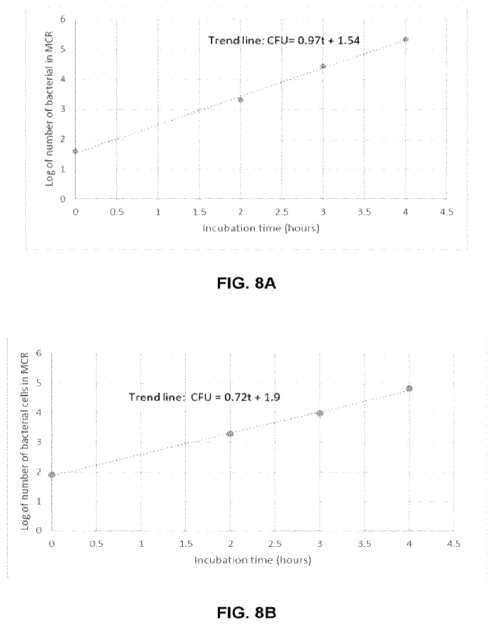

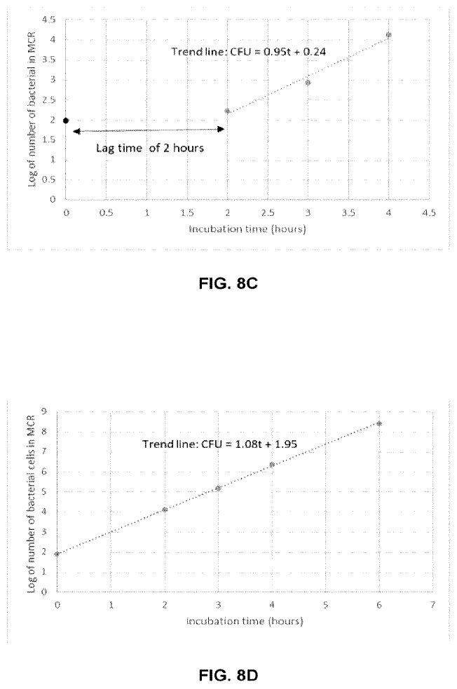

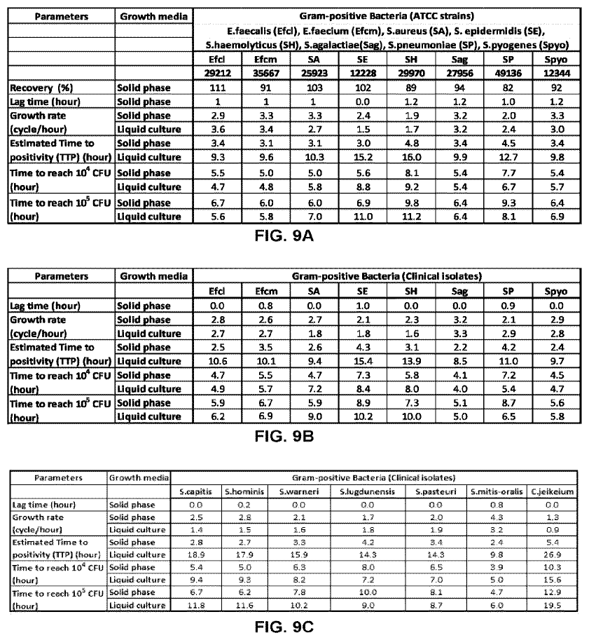

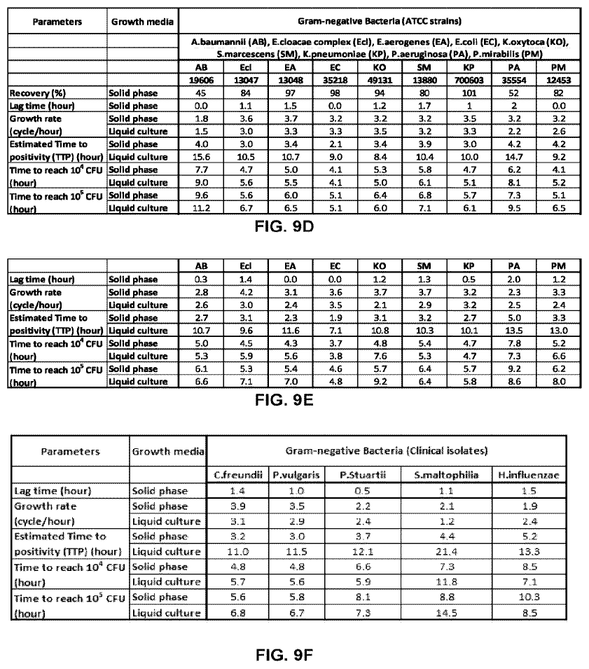

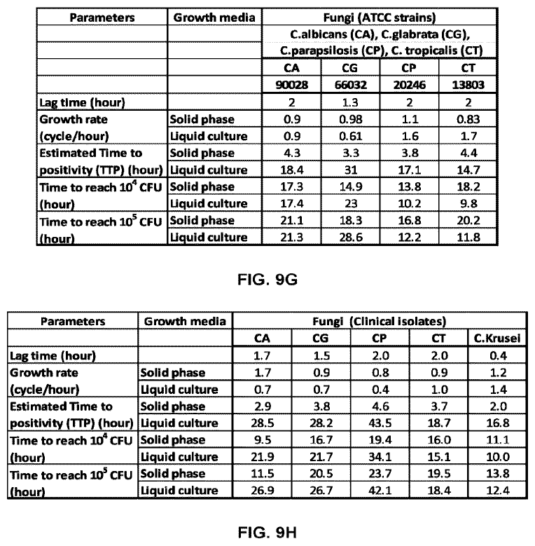

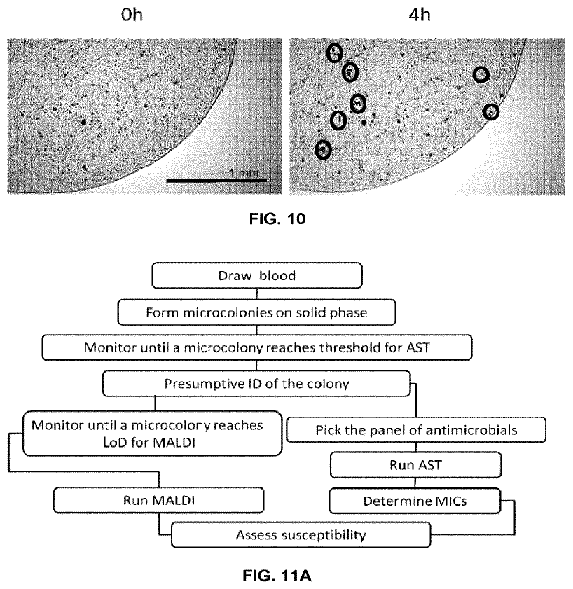

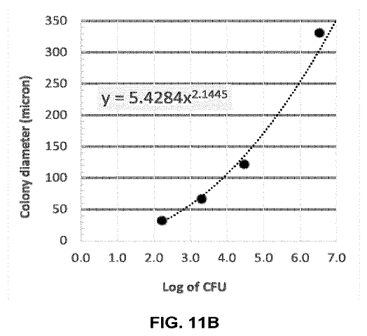

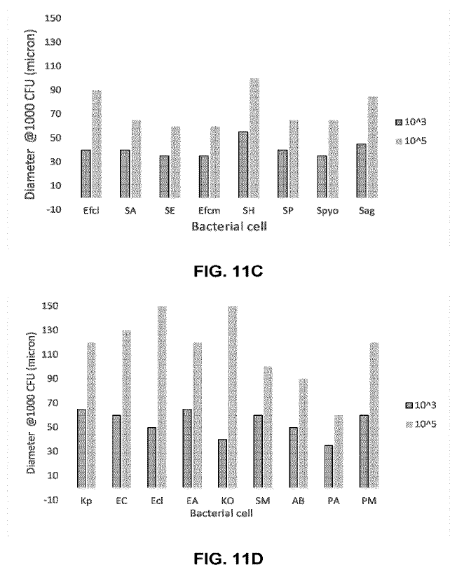

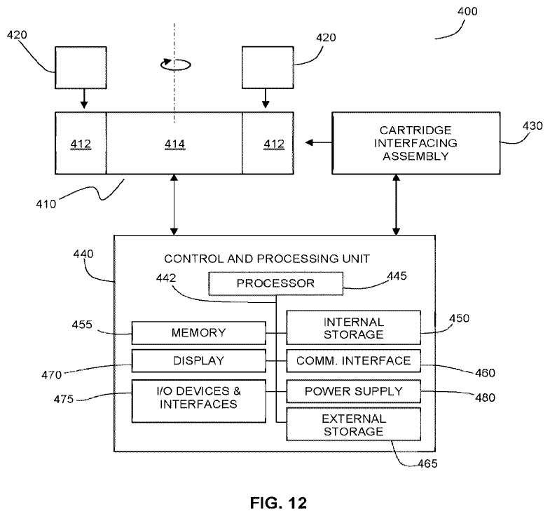

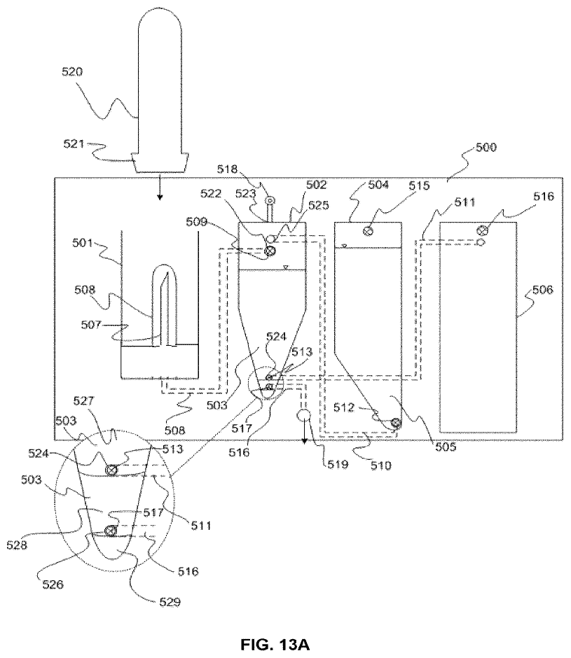

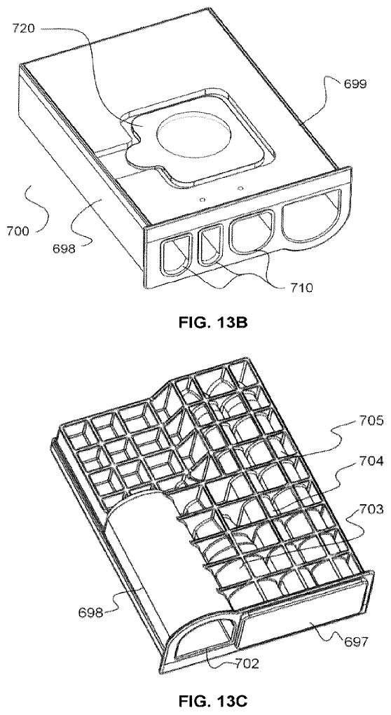

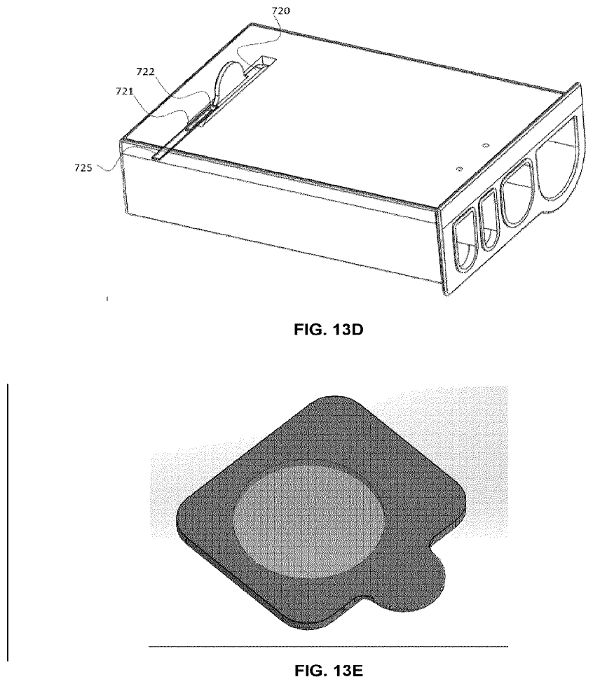

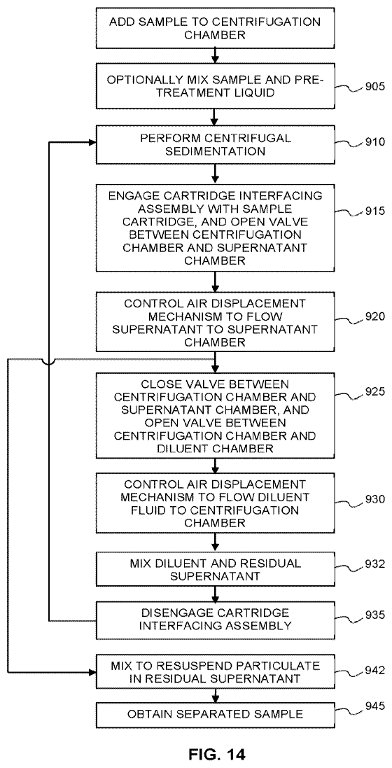

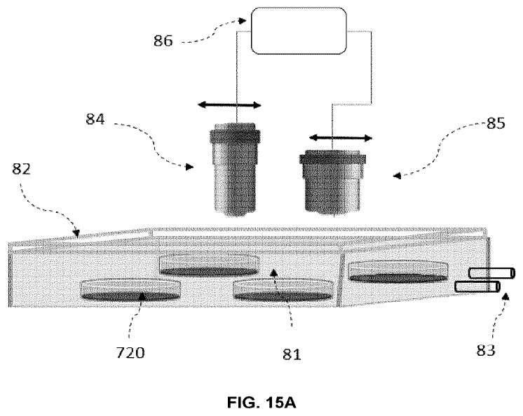

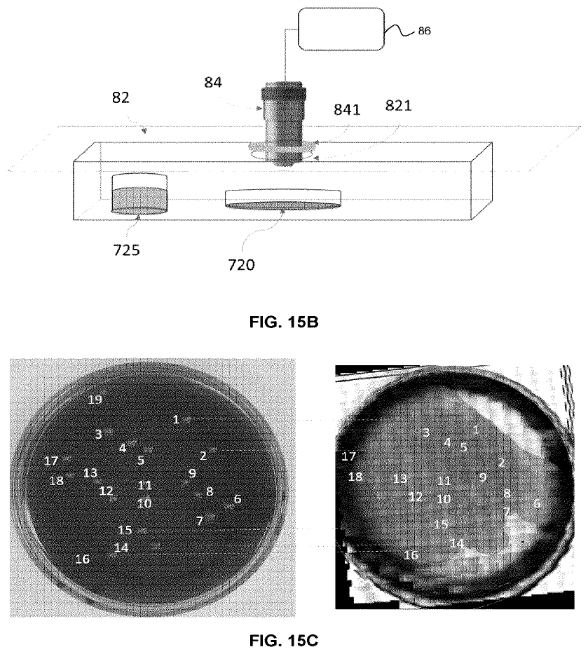

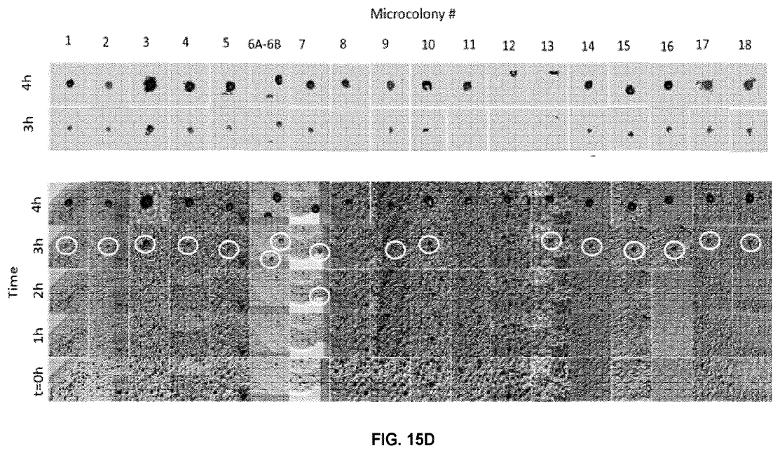

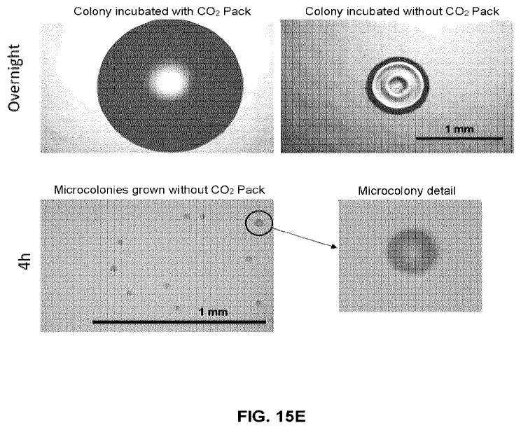

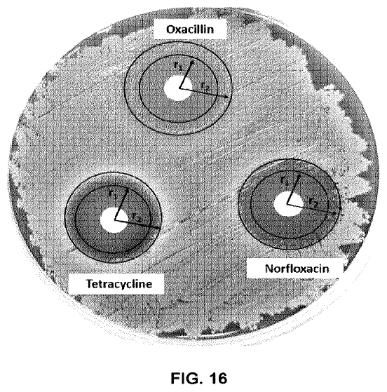

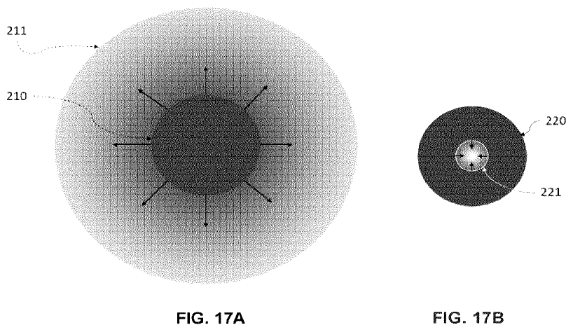

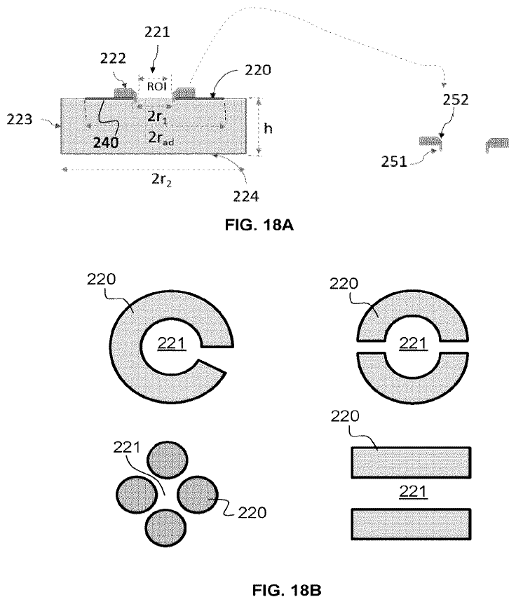

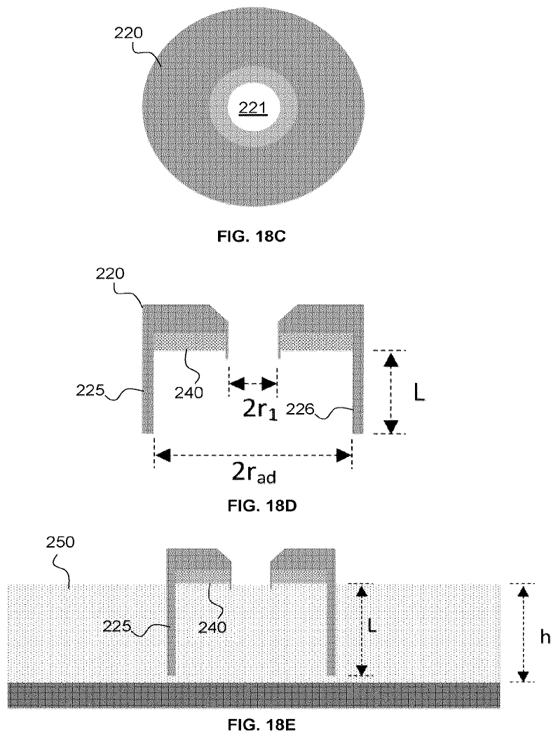

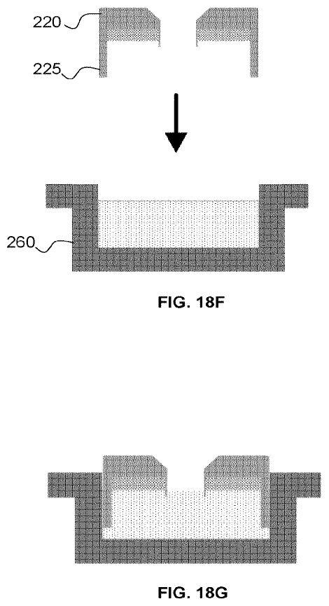

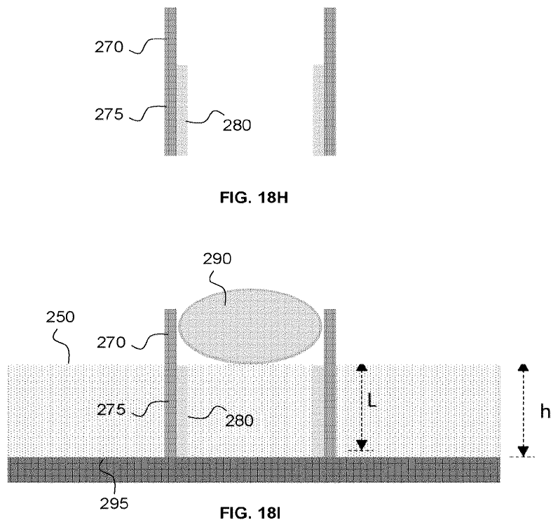

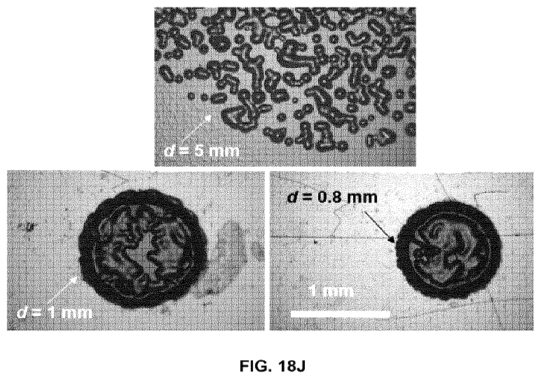

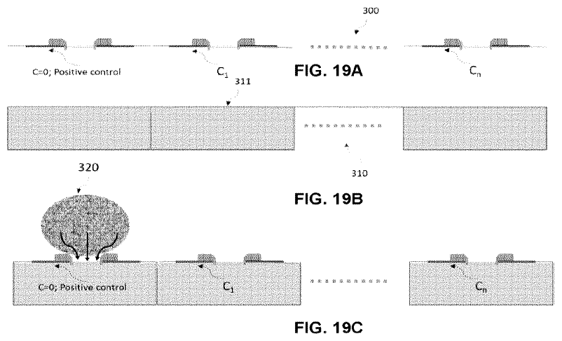

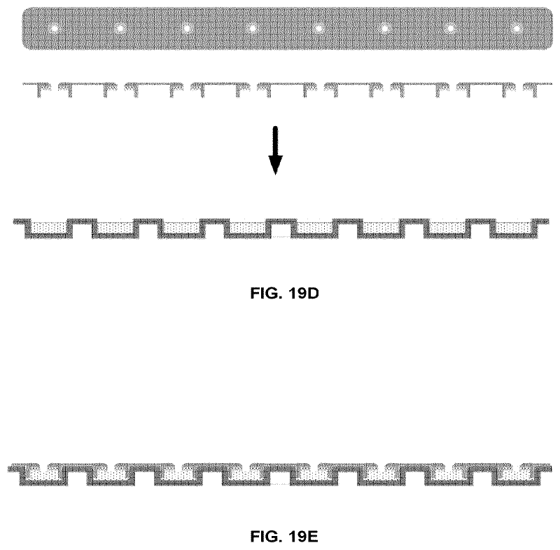

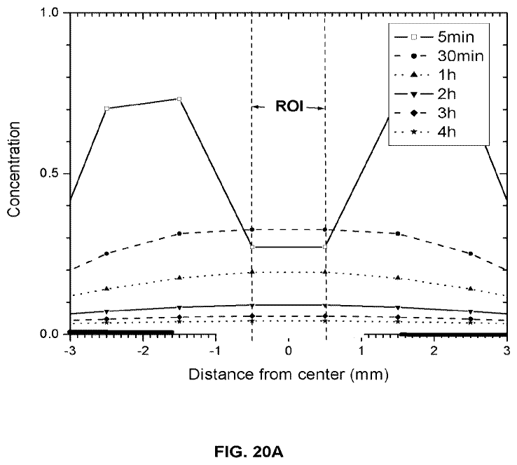

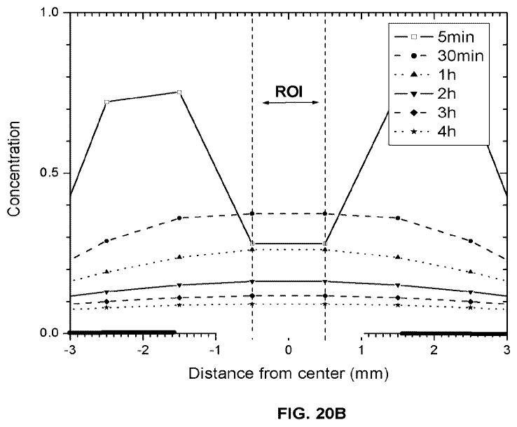

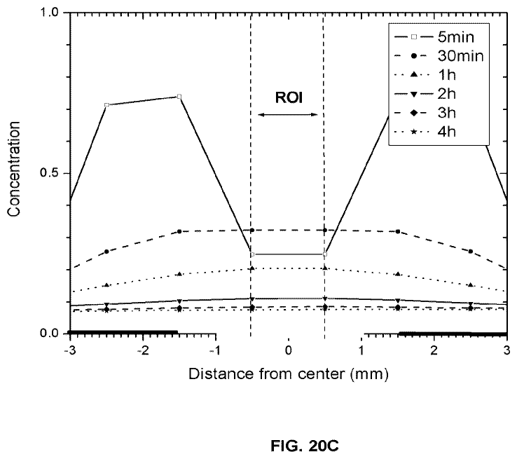

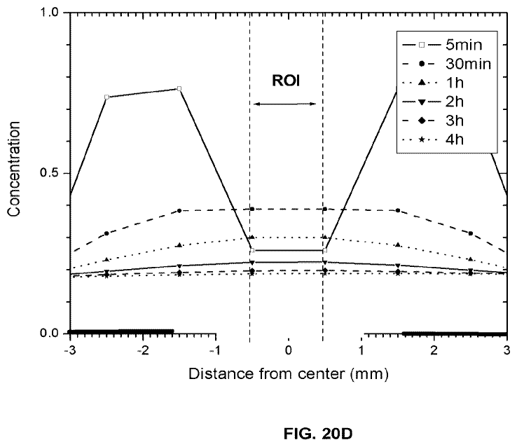

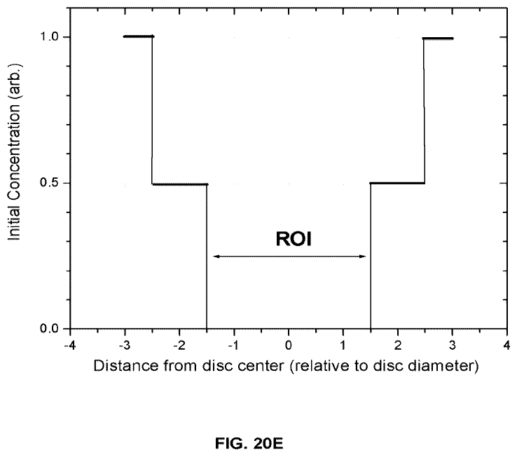

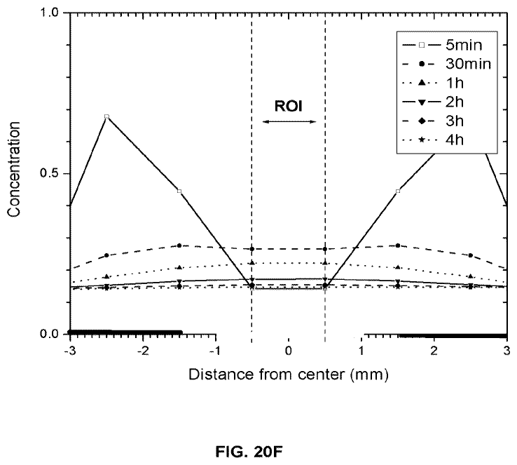

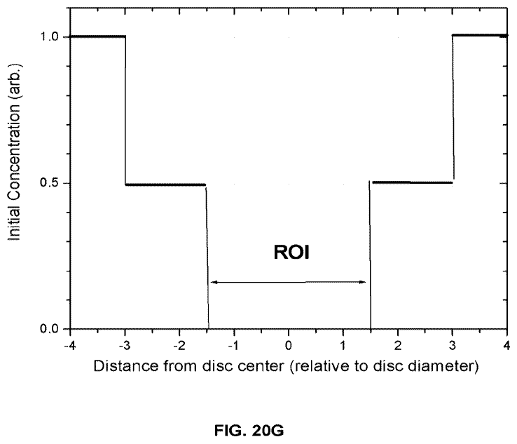

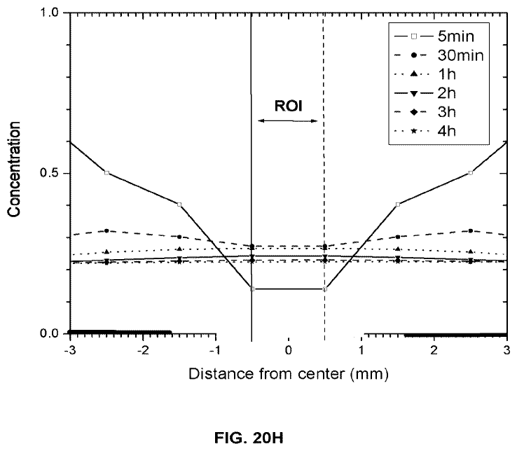

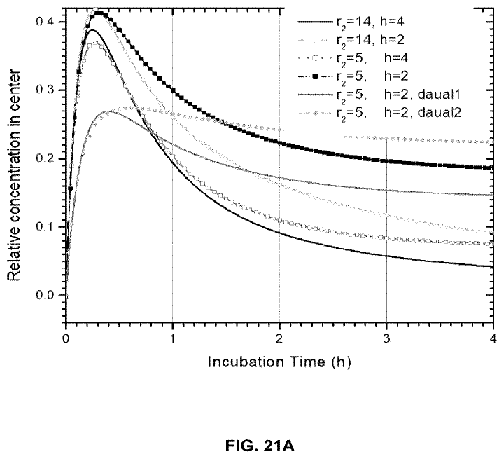

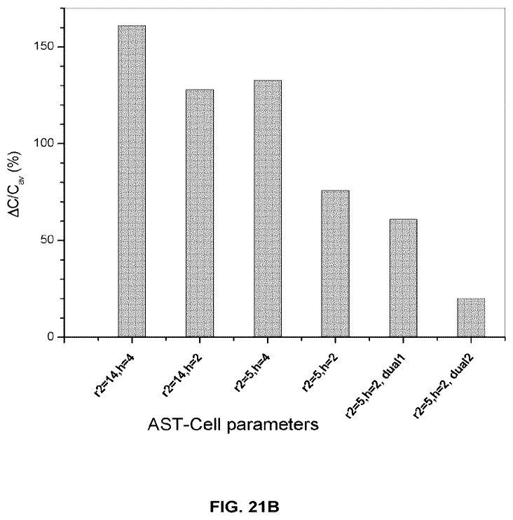

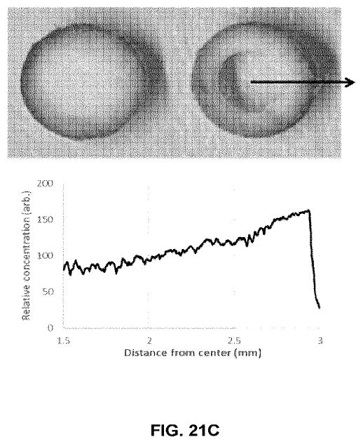

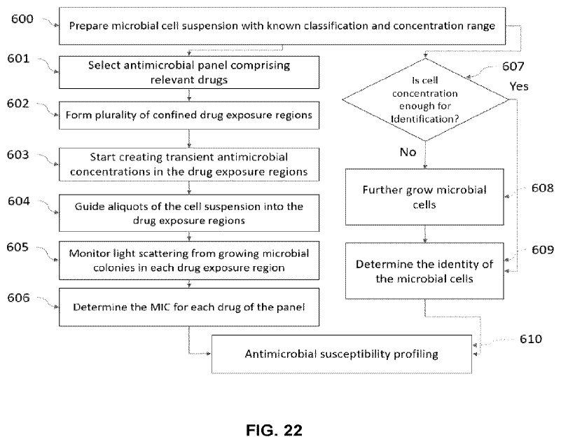

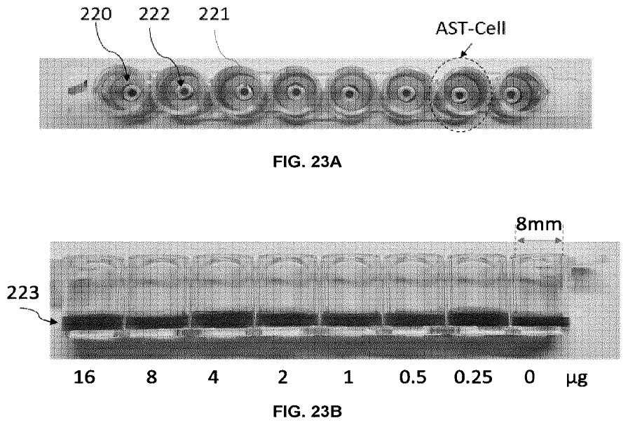

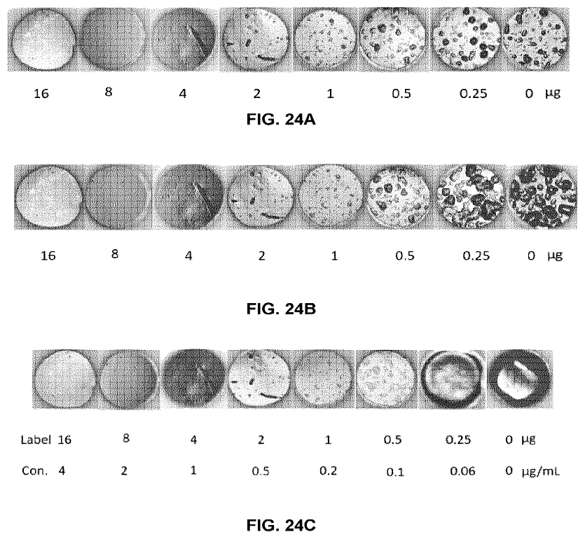

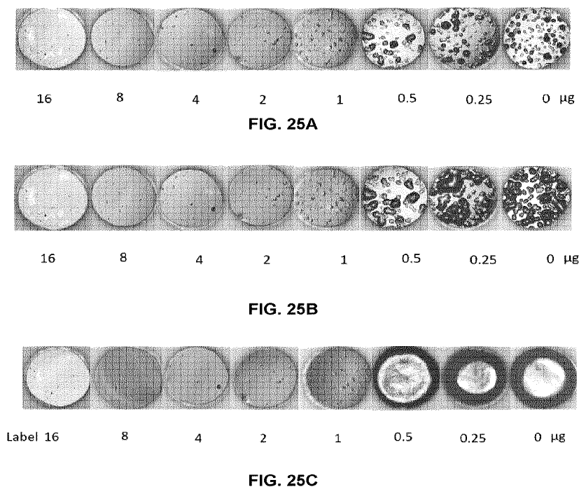

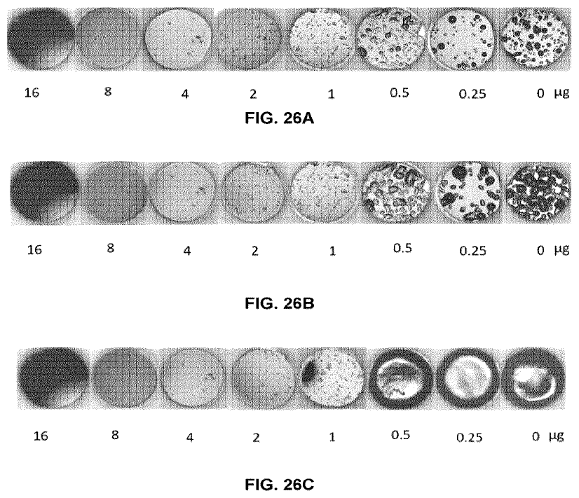

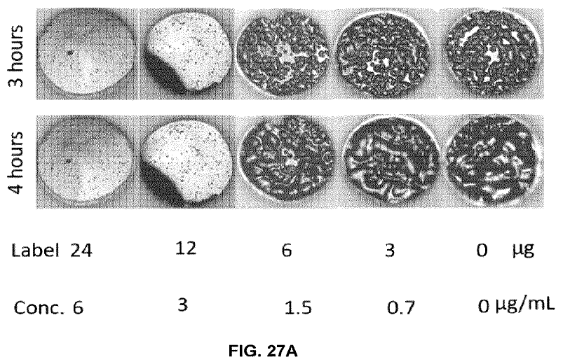

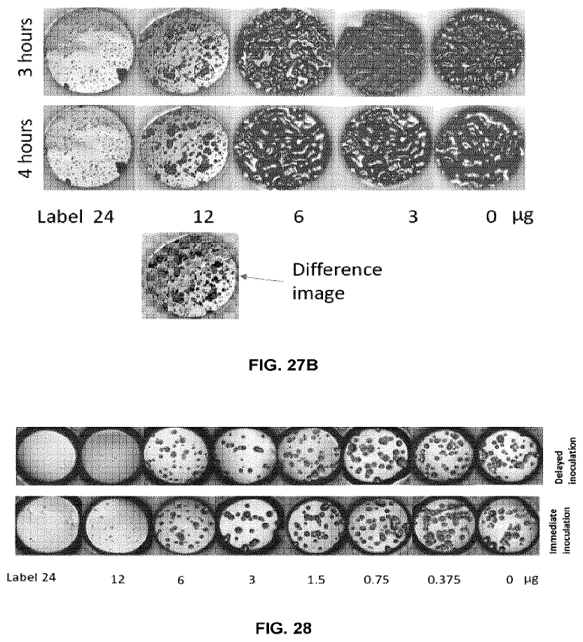

FIG. 5J schematically illustrates another example embodiment for removing the centrifugally exuded liquid from the gel, through a channel, during the steps shown in FIGS. 5A and 5B.

FIG. 5K schematically illustrates a growth chamber for testing an implementation of the embodiment presented in FIG. 5J.

FIG. 5L shows a photo of an experimental realization of the growth chamber of FIG. 5K at different time points after pouring the gel.

FIG. 5M shows a photo of an experimental realization of the growth chamber of FIG. 5K at different time points after centrifugation for 8 minutes at 3200 g and dispensing 100 μL of dye solution at 4 spots and allowing the liquid to settle for 5 minutes.

FIG. 6 illustrates a section of mini-culture regions (MCRs) formed on agar plates after dispensing of 1 μL of microbial cell suspension obtained by centrifugally separating a whole blood sample spiked with Proteus mirabilis (PM), imaged by a bright-field (BF) metallurgical microscope with 5× infinite plan objectives at time points of 0 hour, 2 hours, 3 hours, and 4 hours following incubation. The arrows indicate some of the PM microcolonies which can be visually discerned relative to the blood lysis debris.

FIG. 7 illustrates example steps for differentiating microbial colonies on the MCRs of FIG. 6 from the blood lysis debris via time-lapse image analysis. Imaging data acquired at different time points (0, 2, 3 and 4 hours after seeding) was spatially aligned (registered) with respect to 0 hour image, followed by a subtraction of the 0 hour image. Intensity features present within the 0 hour image were classified as background (blood lysis debris) while intensity features appearing in the subtracted images were classified as foreground microcolonies).

FIG. 8A plots the number of colony-forming units (CFU) of PM bacterial cells recovered after the centrifugal separation and subsequent seeding onto agar of microbial cells from a spiked whole blood sample (as employed in the experiment of FIG. 6 at different time points following seeding the final cell suspension and incubating at 37° C. for 4 hours.

FIG. 8B plots the number of CFU of Staphylococcus epidermidis bacterial cells recovered after the centrifugal separation and subsequent seeding onto agar of microbial cells from a spiked whole blood sample at different time points following seeding the final cell suspension and incubating at 37° C. for 4 hours.

FIG. 8C plots the number of CFU of Pseudomonas aeruginosa bacterial cell recovered after the centrifugal separation and subsequent seeding onto agar of microbial cells from a spiked whole blood sample at different time points following seeding the final cell suspension and incubating at 37° C. for 4 hours.

FIG. 8D plots the number of CFU of Escherichia coli bacterial cell recovered after the centrifugal separation and subsequent seeding onto agar of microbial cells from a spiked whole blood sample at different time points following seeding the final cell suspension and incubating at 37° C. for 6 hours.

FIG. 9A is a table presenting the measured growth parameters of seeded ATCC strains of Gram-positive bacteria, recovered from spiked blood sample via centrifugal separation and subsequent seeding onto agar. The lag time before growth, growth rate, estimated time to positivity and the average time required for the number of cells in a microcolony to reach 104 and 105 CFU are presented for seeded cells growth vs reference growth inside blood culture bottles (liquid culture).

FIG. 9B is a table presenting the measured growth parameters of seeded clinical isolates of Gram-positive bacteria, recovered from spiked blood sample via centrifugal separation and subsequent seeding onto agar. The lag time before growth, growth rate, estimated time to positivity and the average time required for the number of cells in a microcolony to reach 104 and 105 CFU are presented for seeded cells growth vs reference growth inside blood culture bottles (liquid culture).

FIG. 9C is a table presenting the measured growth parameters of additional seeded clinical isolates of Gram-positive bacteria, recovered from spiked blood sample via centrifugal separation and subsequent seeding onto agar. The lag time before growth, growth rate, estimated time to positivity and the average time required for the number of cells in a microcolony to reach 104 and 105 CFU are presented for seeded cells growth vs reference growth inside blood culture bottles (liquid culture).

FIG. 9D is a table presenting the measured growth parameters of seeded ATCC strains of Gram-negative bacteria, recovered from spiked blood sample via centrifugal separation and subsequent seeding onto agar. The lag time before growth, growth rate, estimated time to positivity and the average time required for the number of cells in a microcolony to reach 104 and 105 CFU are presented for seeded cells growth vs reference growth inside blood culture bottles (liquid culture). FIG. 9E is a table presenting the measured growth parameters of seeded clinical isolates of Gram-negative bacteria, recovered from spiked blood sample via centrifugal separation and subsequent seeding onto agar. The lag time before growth, growth rate, estimated time to positivity and the average time required for the number of cells in a microcolony to reach 104 and 105 CFU are presented for seeded cells growth vs reference growth inside blood culture bottles (liquid culture).

FIG. 9F is a table presenting the measured growth parameters of additional seeded clinical isolates of Gram-negative bacteria, recovered from spiked blood sample via centrifugal separation and subsequent seeding onto agar. The lag time before growth, growth rate, estimated time to positivity and the average time required for the number of cells in a microcolony to reach 104 and 105 CFU are presented for seeded cells growth vs reference growth inside blood culture bottles (liquid culture).

FIG. 9G is a table presenting the measured growth parameters of seeded ATCC strains of fungal cells, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 9H is a table presenting the measured growth parameters of seeded clinical isolates of fungi, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 10 illustrates the determination, via optical microscopy, of the positivity of a spiked blood sample for Candida albicans cells (visible inside ovals) after separation from whole blood sample and incubating for 4 hours.

FIG. 11A provides a flow chart illustrating an example method for performing rapid antimicrobial susceptibility testing (AST) on microcolonies.

FIG. 11B illustrates the average diameter versus microcolony cell content plot for E. coli. The plot has been fitted with a power law trendline for enabling the estimation of average microcolony diameters, for example, at 10³ and 10⁵ cell content levels.

FIG. 11C plots the average microcolony diameters at 10³ and 10⁵ cell content levels for various pathogenic gram-positive bacteria prevalent in blood stream infection.

FIG. 11D illustrates the average microcolony diameters at 10³ and 10⁵ cell content levels for various pathogenic gram-negative bacteria prevalent in blood stream infection.

FIG. 12 schematic of a system for performing automated centrifugation and washing with an integrated fluidic processing cartridge.

FIGS. 13A to 13E illustrate an example integrated fluidic processing cartridge configured for extraction of a sample directly from a collection tube, subsequent centrifugation and washing, to obtain a concentrated and purified suspension of microbial cells.

FIG. 14 provides a flow chart illustrating an example method for performing automated centrifugation and washing.

FIG. 15A shows a schematic of an example system for incubating the growth chamber, monitoring growth of microbial cells for the detection of viable microbial microcolonies and classifying the microcolony cells as belonging to a given microbial cell class. Objective lenses with low magnification are employed to increase the observed area and can increase temporal resolution for time-lapse imaging.

FIG. 15B shows a schematic of another example system for incubating the growth chamber, monitoring growth of microbial cells for the detection of viable microbial microcolonies.

FIG. 15C illustrates the comparison of E. coli microcolonies/colonies detected on a solid phase growth media 4 hours (right) and 20 hours (left) after sample seeding and incubation at 35 C. The image on the right is the result of mosaic stitching of 448 aligned/registered microscopic images taken by 5× brightfield objective in an automated system similar to the one schematically presented in FIG. 15B. The image on the left was taken by a conventional camera.

FIG. 15D illustrates the 18 microcolonies identified on the plate shown in FIG. 15C, as detected during 4 hours of incubation via time-lapse image processing. As can be seen in the figure, 15 out of 18 microcolonies, shown by white circles, were detected at 3 hours, and one microcolony (microcolony 7) was detected at 2 hours.

FIG. 15E shows the morphology of Streptococcus pneumoniae colonies respectively grown in the presence (up-left) and absence (up-right) of a CO₂ pack for overnight. The image is taken with 5× brightfield objective. A typical microcolony of Streptococcus pneumoniae formed after 4 hours of incubation in the absence of CO₂ pack is presented at the bottom-left corner. The image has been taken by 10× brightfield objective. The zoomed image of the microcolony, presented in bottom right corner, is more similar to the overnight colony formed at the presence of CO₂ pack.

FIG. 16 is a photograph of a blood agar gel on which Staphylococcus aureus cells have been streaked. Commercial paper disks (Hardydisk™), respectively, impregnated with Oxacillin 1 ug/mL, Tetracycline 30 μg/mL and Norfloxacin 10 ug/mL, have been placed on the solid phase growth media and photo has been taken after overnight incubation. The measure r1 shows visible absence of microbial lane up to a distance from the center and represents zone of inhibition. Beyond this distance, sparse lane is observed up to a distance r2, beyond which the lane is full.

FIGS. 17A and 17B schematically compare the diffusion of an antimicrobial agent from an impregnated disk which has been placed on the gel surface. The cases of conventional disk diffusion AST is shown in FIG. 17A, while an example annular disk diffusion AST embodiment is shown in FIG. 17B. The arrows represent the directions of diffusion of the antimicrobial agent that are respectively relevant for disk diffusion antimicrobial susceptibility testing and annular-disk diffusion antimicrobial susceptibility testing.

FIG. 18A schematically illustrates an example annular disk diffusion device (a “LD-AST unit”), indicating the parameters r₁, r_{ad} and h.

FIGS. 18B-181 illustrate example embodiments of solid support structures for performing lateral diffusion AST.

FIG. 18J shows a set of images demonstrating capability of a guiding ring, cut from a 100 μm thick Thermoplastic polyurethane (TPU) sheet, to localize and concentrate microbial cells within the inner aperture of the ring, when the ring is placed on a gel surface and a microbial cell suspension is dispensed over the aperture.

FIGS. 19A-19E schematically represents the assembly of annular disk diffusion “LD-AST units” into an array. The annular disks impregnated with an antimicrobial at different concentration levels are supplied with interaction rings and assembled in a strip (FIG. 19A). A complementary strip (FIG. 19B) includes assembly of sealed microwells having agar gels. During the assay two components are attached and aliquots of the sample are dispensed inside the interaction rings (FIG. 19C).

FIG. 20A plots the simulated concentration on the surface of the gel across a centerline passing through an annular diffusion disk (the disk is indicated by the black strip). The gel thickness is taken to be h=4 mm and its radius is r₂=14 mm. The cross-section of the annular disk, having the inner radius r₁=1.5 mm and the outer radius r_{ad}=3 mm, is illustrated by thick line.

FIG. 20B plots the evolution of the simulated concentration on the surface of the gel across a centerline passing through the annular disk, indicated by the black strip. The gel thickness is taken to be h=2 mm and its radius r₂=14 mm.

FIG. 20C plots the evolution of the simulated concentration on the surface of the gel across a centerline passing through the annular disk, indicated by the black strip. The gel thickness is taken to be h=4 mm and its radius r₂=5 mm.

FIG. 20D plots the evolution of the simulated concentration on the surface of the gel across a line passing through the annular disk, indicated by the black strip. The gel thickness is taken to be h=2 mm and its diameter r₂=5 mm.

FIG. 20E plots an example impregnated concentration profile on annular disk, referred to below as dual1.

FIG. 20F plots the evolution of the simulated concentration on the surface of the gel across a centerline passing through the annular disk according to the impregnated concentration profile shown in FIG. 20E. The gel thickness is taken to be h=2mm and its radius r₂=5 mm.

FIG. 20G plots an example impregnated concentration profile on annular disk, referred to below as dual2.

FIG. 20H plots the evolution of the simulated concentration on the surface of the gel across a centerline passing through the annular disk according to the impregnated concentration profile shown in FIG. 20G. The gel thickness is taken to be h=2 mm and its radius r₂=5 mm.

FIG. 21A represents the simulated temporal behavior of drug concentration at the center of the region of interest for the LD-AST units of FIGS. 20A to 20H. The plots labeled with (r₂=14 mm, h=4 mm), (r₂=14 mm, h=2 mm), (r₂=5 mm, h=4 mm), (r₂=5 mm, h=2 mm), (r₂=5 mm, h=2 dual1) and (r₂=5 mm, h=2 dual2), respectively correspond to FIGS. 20A, 20B, 20C, 20D, 20F, and 20G. r-horizontal dimension of gel; h-gel thickness.

FIG. 21B plots the relative change in the concentration at the center of the region of interest for the curves of FIG. 21A between during the period of reaching max concentration at ~0.5 h and 4 h after placing the annular disk on the gel. The plots labeled with (r₂=14 mm, h=4 mm), (r₂=14 mm, h=2 mm), (r₂=5 mm, h=4 mm), (r₂=5 mm, h=2 mm), (r₂=5 mm, h=2 dual1) and (r₂=5 mm, h=2 dual2), respectively correspond to FIGS. 20A, 20B, 20C, 20D, 20F, and 20G.

FIG. 21C presents the qualitative and quantitative concentration profile of a dye solution deposited on an annular disk according to the method of Example 9B.

FIG. 22 represents a flowchart for rapidly performing annular-disk diffusion AST.

FIG. 23A is an overhead photograph of a strip for running LD-AST at 8 drug concentration levels. The photo corresponds to the case of thin strips, i.e. 50 μL of gel in each microwell.

FIG. 23B is a photograph showing a side view of the strip shown in FIG. 23A. The photo corresponds to the case of thin strips, i.e. 50 μL of gel in each microwell.

FIGS. 24A-24C show overhead images of the regions of interest (ROIs) of the strip shown in FIGS. 23A and 23B (low gel volume of ~50 μL; gel thickness of ~1 mm) after incubating for 3 hours, 4 hours, and overnight incubation, respectively. The antibiotic is Norfloxacin and the microbial cell is Escherichia coli.

FIGS. 25A-25C show overhead images of the ROIs of the mid-thick (gel volume ~150 μL; gel thickness ~3 mm) strip after incubating for 3 hours, 4 hours, and overnight incubation, respectively. The antibiotic is Norfloxacin and the microbial cell is Escherichia coli.

FIGS. 26A-26C show overhead images of the ROIs of the thick (gel volume=350 μL; gel thickness ~7 mm) strip after incubating for 3 hours, 4 hours, and overnight incubation, respectively. The antibiotic is Norfloxacin and the microbial cell is Escherichia coli.

FIG. 27A shows overhead images of the ROIs of the “thin strip” type LD-AST (shown in FIGS. 23A and 23B) after incubating for 3 and 4 hours. The antibiotic is Vancomycin and the microbial cell is Staphylococcus aureus.

FIG. 27B shows overhead images of the ROIs of the “mid-thick strip” type LD-AST after incubating for 3 and 4 hours. The antibiotic is Vancomycin and the microbial cell is Staphylococcus aureus.

FIG. 28 shows overhead images of the ROIs of the “mid-thick strip” type LD-AST after incubating for 4 hours following the inoculation. The antibiotic is Vancomycin and the microbial cell is Staphylococcus aureus.

FIG. 29 shows overhead images of the regions of interest (ROIs) of the “thin strip” type LD-AST after incubating for 3 hours 4 hours and overnight while testing the susceptibility of Amphotoricin B against Candida albicans.

FIG. 30 show overhead images of the regions of interest (ROIs) of the “thin strip” type LD-AST after incubating for 3 hours and 4 hours, respectively, for clean cell suspension (top) and the positive blood culture diluted by 1000-fold (bottom).

The antibiotic is Oxacillin and the microbial cell is Methicillin-resistant Staphylococcus aureus (MRSA 111 with not strong resistance).

FIG. 31A shows the growth pattern of MRSA-110 in the wells of a commercial broth microdilution AST plate. The indicated concentrations are in μg/mL. The abbreviations are as the following: CHL=Chloramphenicol, DAP=Daptomycin, GEN=Gentamicin, LZD=Linezolid, RIF=Rifampin, SXT=Trimethoprim/Sulfamethoxazole, SYN=Quinupristin/Dalfopristin, TET=Tetracycline, ERY=Erythromycin, OXA+=Oxacillin+2% NaCl, AMP=Ampicillin, PEN=Penicillin, VAN=Vancomycin, LEVO=Levofloxacin, TGC=Tigecycline, MXF=Moxifloxacin, CLI=Clindamycin, STR=Streptomycin, CIP=Ciprofloxacin, NIT=Nitrofurantoin, DT1=D Test 1, DT2=D Test 2, FOXS=Cefoxitin screen, NEG=Negative control and POS=Positive control.

FIG. 31B shows the growth pattern of MRSA-110 in the wells of a 96 well microplate where the LD-AST assay is performed. The abbreviations are the same as FIG. 31A.

FIG. 32 shows a comparison between the results of LD-AST and standard broth microdilution AST for selected drug-bug combinations. The abbreviations for the microbial cells are the following: Staphylococcus aureus (SA), Methicillin-resistant Staphylococcus aureus (MRSA), Acinetobacter baumannii (AB), Escherichia coli (EC), Pseudomonas aeruginosa (PA), Proteus mirabilis (PM), Klebsiella pneumoniae (KP), carbapenem-resistant Enterobacteriaceae Klebsiella pneumoniae (CRE).

### DETAILED DESCRIPTION

Various embodiments and aspects of the disclosure will be described with reference to details discussed below. The following description and drawings are illustrative of the disclosure and are not to be construed as limiting the disclosure.

Numerous specific details are described to provide a thorough understanding of various embodiments of the present disclosure. However, in certain instances, well-known or conventional details are not described in order to provide a concise discussion of embodiments of the present disclosure.

As used herein, the terms “comprise” and “comprising” are to be construed as being inclusive and open ended, and not exclusive. Specifically, when used in the specification and claims, the terms “comprise” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As used herein, the phrase “intact cell” refers to a microbial cell containing nucleic acids, proteins or intracellular contents of interest, where the microbial cell is separable via a separation method such as, but not limited to, centrifugal separation, filtration, microfluidic separation, or immunomagnetic separation.

As used herein, the phrase “sample” refers to a liquid or suspension that contains, may contain, or is suspected of containing one or more microbial cells. Non-limiting examples of samples include body fluids such as urine, lymph fluid, cerebrospinal fluid, blood (e.g. whole blood, blood culture, and plasma), sputum and saliva. Other examples of samples include homogenized tissue suspensions, including, but not limited to, stool, homogenized suspensions of muscle tissue, brain tissue and liver tissue. A sample may be processed or unprocessed and may optionally include one or more reagents or growth media. In the case of a blood culture sample (a sample containing growth media and whole blood), the blood culture sample may be a blood culture sample having been deemed positive for the presence of microbial cells via a detection modality (e.g. via an automated blood culture system), a mid-culture blood culture sample for which the presence of microbial cells is suspected based on measurements made via one or more mid-culture detection modalities, or mid-culture blood culture sample for which no initial detection results are available.

As used herein, the phrase “blood cells” refers to mammalian cells present in blood, including, but not limited to, red blood cells (erythrocytes), white blood cells (leukocytes) and blood platelets (thrombocytes).

As used herein, the phrase “blood sample” refers to any sample comprising one or more blood cells. Non-limiting examples of blood samples include whole blood samples, blood culture samples, buffy coat samples and platelet samples.

As used herein, the phrase “whole blood” or “whole blood sample” refers to mammalian blood comprising blood plasma and blood cells. “Whole blood” or “a whole blood sample” may include one or more reagents, such as anticoagulation reagents. For example, whole blood may be collected in a sample bottle that may include one or more reagents such as, but not limited to, anticoagulants including SPS (sodium polyanethole sulfonate), EDTA (ethylenediaminetetraacetic acid), sodium citrate and heparin.

As used herein, the phrase “selective lysis” refers to a blood lysis reagent or lysis process whereby the fraction of microbial cells that remain viable following lysis exceeds the fraction of eukaryotic cells that remain viable following lysis, where the eukaryotic cells are associated with the subject from which the sample was collected. As used herein, the phrases “microbial cell” and “micro-organism” comprises bacteria (e.g. Gram-positive and Gram-negative bacteria, as well as bacterial spores) and unicellular fungi (such as yeast and molds).

As used herein, the phrase “eukaryotic cell” refers to cells originating from an eukaryotic organism excluding fungi, such as animals, in particular animals containing blood, comprising invertebrate animals such as crustaceans and vertebrates. As used herein, “vertebrates” comprise both cold-blooded animals (fish, reptiles, amphibians) and warm-blooded animals (birds and mammals).

As used herein, the phrase “effective buffer concentration”, when used with reference to a mixture formed by mixing a volume of a sample with a volume of a blood lysis reagent, where the blood lysis reagent includes a buffer system, refers to the product of the buffer concentration of the blood lysis reagent and a ratio formed by dividing the volume of the blood lysis reagent by the sum of the volume of the blood lysis reagent and the volume of the sample. The effective buffer concentration represents the contribution of the blood lysis reagent to the buffer system in the final mixture (i.e. the dilution factor applied to the buffer concentration of the blood lysis reagent) and may be different than the actual buffer concentration in the final mixture due to buffering components present in the sample.

As used herein, the phrase “separation process” refers to a process suitable for separating and optionally concentrating microbial cells. Non-limiting examples of separation processes include centrifugation, filtration, immunomagnetic separation and microfluidic separation.

As used herein, the phrase “cell suspension” refers to an aqueous medium that contains microbial cells.

As used herein, the terms “colony” and “microcolony” refer to a multiplicity or population of microorganisms that lie in close proximity to each other, that lie on a surface, and that are the clonal descendants, by in situ replication, of a single ancestral microorganism. In general, a “colony” is visible to the human eye and is typically greater than about 50 μm, 60 μm, 80 μm, or 100 μm, in diameter. However, as used herein, unless otherwise stated, the term “colony” is meant to include both colonies having a diameter of 100 μm or more, and the term “microcolony” is meant to refer to a colony having a diameter less than 100 μm.

Various example embodiments of the present disclosure address the aforementioned shortcomings with conventional approaches to microbial growth and antimicrobial susceptibility testing (AST). As explained in detail below, many of the example embodiments of the present disclosure employ an integrated fluidic cartridge to facilitate the separation of microbial cells and the subsequent growth of microbial colonies in situ within the integrated fluidic cartridge. In many example embodiments, the microbial cells are separated and contacted with a solid phase growth media for subsequent colony growth while maintaining at least a portion of the integrated fluidic cartridge in a closed configuration.

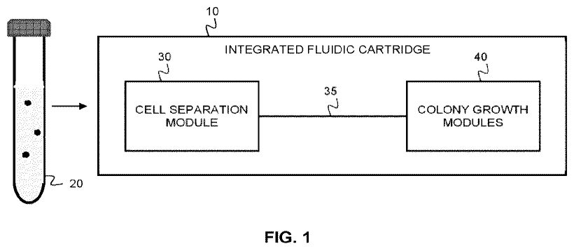

Referring now to FIG. 1, an example integrated fluidic cartridge (device) for performing microbial colony growth is schematically illustrated. A sample 20 may be introduced into the integrated fluidic cartridge 10, which includes a cell separation module (a portion or region of the integrated fluidic cartridge) 30 and a colony growth module (another portion or region of the integrated fluidic cartridge) 40. The integrated fluidic cartridge 10 facilitates the separation of microbial cells from other components of a sample, such as eukaryotic cells (e.g. host cells from a host subject) such that the separated microbial cells are separated in an intact and viable form (capable of cell division). The separated microbial cells may be provided, an optionally concentrated, in a liquid medium (e.g. saline or another buffer suitable for maintaining microbial cell viability), thereby providing a microbial cell suspension.

The cell suspension is subsequently introduced, e.g. via one or more fluidic conduits 35, into the colony growth module 40 and contacted therein with (e.g. seeded on) a solid phase growth media, examples of which are described in detail below. The colony growth module 40 is configured to permit the monitoring (e.g. via optical or electrical modalities) of colonies that grow on the solid phase growth media. The integrated fluidic cartridge 10 may subsequently be incubated at an appropriate temperature and environment for promoting growth (e.g. 35-38° C.) while permitting monitoring of the growth of microbial colonies.

As noted above, the microbial cells may be separated and contacted with a solid phase growth media for subsequent colony growth while maintaining at least a portion of the integrated fluidic cartridge in a closed configuration. It will be understood that the phrases “closed” and “closed state” refer to a capability or configuration of an internal region of the integrated fluidic cartridge to be at least temporarily brought into a state that prohibits the ingress (entrance or introduction) of external microbial cells within an interior region of the integrated fluidic device, thereby avoiding contamination of microbial cells that are separated from the sample and grown in colonies within the colony growth module. An inner region of an integrated fluidic cartridge may be brought into a closed state by actuation of suitably located valves.

In some example embodiments, an internal region of an integrated fluidic cartridge may be in a closed state while permitting gas communication of the internal region with an external gas source or external environment, for example, through a filter that prohibits that passage of microbial cells. One internal region of a fluidic cartridge may be configured in a closed state while other internal regions of the fluidic cartridge reside in a non-closed state. As explained in some example embodiments below, at least a portion of an internal region of an integrated fluidic cartridge that resides in a closed state may be opened to provide external access to microbial cells (e.g. microbial colonies) residing therein, for example, after sufficient microbial growth for further testing has been detected.

It will be understood that the integrated fluidic cartridge 10 may be configured such that the cell separation module performs separation of viable microbial cells, prior to the contact of the viable microbial cells with the solid phase growth media, according to a manual method, a semi-automated method, or a fully-automated method, such that the resulting microbial suspension may be transported to the colony growth module while maintaining the microbial cell suspension in a closed environment within the integrated fluidic cartridge.

For example, in some example embodiment, the microbial cells may be separated via an automated lysis-centrifugation process, using an integrated fluidic cartridge such as the cartridge shown in FIGS. 13A-13E (described in detail below as providing an example of the cell separation module of an integrated fluidic device) or a variation thereof, such that the suspension automatically to contact the solid phase growth media. In an alternative example implementation, the process may be fully automated within a closed integrated cartridge, which may be beneficial in avoiding the introduction of contaminants when transferring the separated microbial cells to the solid phase growth media. In another example embodiment, the microbial cells may be separated using a filter, where the integrated fluidic cartridge includes a plunger that is either manually or robotically actuated.

It will be understood that although the example integrated cartridge shown in FIGS. 13A-13E employs automated lysis-centrifugation for performing separation of viable microbial cells, such an integrated cartridge may be modified to include an alternative separation modality, such as, but not limited to, filtration, immunomagnetic separation, or other separation modalities including, but not limited to, cell sorting techniques, such as flow cytometric cell sorting, electrical cell sorting or microfluidic cell sorting. Furthermore, while some of the example embodiments disclosed herein involve the separation of viable microbial cells from a blood sample, it will be understood that a wide variety of sample types may be employed, as per the definition of the phase “sample” provided above.

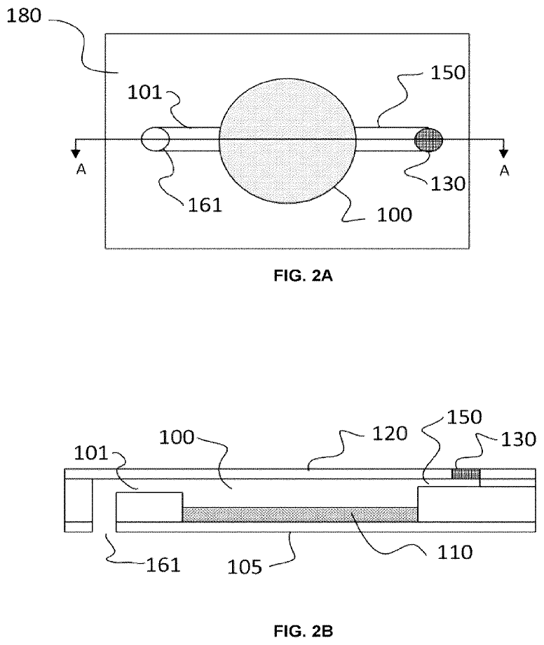

The colony growth module includes a growth chamber that facilitates both the growth and monitoring of microbial colonies on the solid phase growth media. An example embodiment of such a growth module with an internal growth chamber is illustrated in FIGS. 2A-2B. Referring first to FIG. 2A (plan view) and 2B (section view A-A), the colony growth module 180 includes a growth chamber 100 with a lower inner wall 105 having a layer of solid phase growth media 110 formed thereon, an upper wall 120, a conduit 101 fluidically connected, for example, to the cell separation module via path 161 (or 35 in FIG. 1). The growth chamber 100 may also be fluidically connected via conduit 150 to an air vent 130 or such an air vent may be formed in upper wall 120 of the chamber.

In some example embodiments, the upper wall 120 may contain a gas permeable section for allowing air exchange between the chamber and the external environment during the microbial cell growth. The gas permeable section may be, for example, a gas permeable membrane. An example of a suitable gas permeable membrane includes, but is not limited to, a polyurethane membrane. These example membranes enable a sufficient rate of gas exchange with ambient environment to facilitate cell growth on the solid phase, while sustaining a contaminant-free environment within the chamber.

The solid phase growth media 110 is suitable for providing the microbial cells with an appropriate source of growth media. Non-limiting examples of solid phase growth media include conventional agar, gelatin, guar gum, Xanthan gum, having suitable growth nutrients. In some example implementations, the solid phase growth media may be chromogenic according to the type of microbial cell. In some embodiments chromogenic or fluorogenic substrate may be added to the agar media for identifying the microorganism by specific or non-specific staining of the colonies, as described, for example, in European Patent Application No. EP1088896A2.

As explained in further detail below, the solid phase growth media may be in a dry or partially dry format such that that a liquid component from the cell suspension is absorbed (at least partially) upon contact.

It will be understood that the microbial cell suspension can be introduced into the growth chamber according to a number of different example implementations. For example, the cell suspension may be flowed into the chamber and over the solid phase growth media by, for example, positive pressure from an upstream location or negative pressure from a downstream location (example via path 150 of FIG. 2B) and via the actuation of appropriately placed valves and pumps. In some example implementations, physical structures such as barriers that impede or reduce fluid flow may be provided within the growth chamber in order to facilitate a suitable spreading of the cell suspension to distribute the microbial cells over the surface of the solid phase growth media.

It will be understood that in some example implementations involving the growth of a small number of colonies and/or the growth of colonies to a limited size, the growth chamber may be closed in the absence of a gas-permeable region. For example, in some of the example embodiments described below, a sufficient volume of required atmosphere such as ambient air or 5% CO₂ may be enclosed within the colony chamber for microbial respiration in the absence of a gas-permeable region. In alternative example embodiments, the growth chamber may be in gas communication, through a filter that serves as a barrier to external microbial cells, with an oxygen source (e.g. the external environment) to facilitate the provision of continuous or intermitted oxygen to the chamber. For example, the growth chamber may be in gas communication, through an intermediate filter that prohibits the entry of external microbial cells, with an external port that is vented to the atmosphere or to a pressurized or pneumatic gas source. The gas communication with the external oxygen source may be controlled via an intermediate valve.

In one example embodiment, the upper wall 120 may include an optically transparent or transmissive region (e.g. a window) through which the growth of colonies can be inspected (e.g. visually or via an imaging device such as a camera). In another embodiment, the upper wall 120 is in the form of a separable lid, which can be occasionally removed for imaging or visually interrogating the gel surface for the growth of microbial colonies.

The growth chamber 100 may optionally include one or more indicators that are capable of detecting metabolic activity of the growing colonies via the colorimetric sensor detection of volatile organic compounds produced by the microorganism.

Non-limiting examples of suitable indicators has been disclosed in US Patent Publication No. 20150099694. Such indicators may permit the identification of the cells in the colony with some level of taxonomic granularity (e.g. Gram status, family, genus, species, strain). In one example embodiment, the one or more indicators may provide information similar to that of a Gram-stain test, permitting the selection of a suitable antibiotic susceptibility testing panel in a non-destructive manner without perturbing or sacrificing any of growing colonies.

In some example embodiments, the colonies may be identified in a non-destructive manner by providing the solid phase growth media with indicators. Non-limiting examples of suitable indicators include chromogenic or fluorogenic substrates, biochemical dyes, pH indicators, as described, for example, in United States Patent Publication No. 2012/0295299A1.

In some example implementations, the gas permeable membrane or another portion of the structure enclosing the growth chamber may be formed from or may include a transparent material (such as, for example, polyurethane) and may extend over the spatial region associated with the growth media, as illustrated in the example embodiment shown in FIG. 2B, thereby permitting the observation of colony growth. The observation can be performed, for example, visually or using an optical detection (e.g. imaging) system, such as phase-contrast or dark-field microscopy. Alternatively, the colonies may be illuminated from backside by the collimated beam of a monochromatic light source, such as laser, and the optical scattering pattern may be inspected or processed to perform microbial cell identification or to determine a class of microbial cells of a given colony, as described further below.

It will be understood that different detection modalities will have different limits of detection for colony growth on solid phase growth media. For example, in the direct microscopic monitoring method, the limit of detection in terms of number of cells per colony can be in the order of 10³. For example, Yoshiakiet al. [Colony fingerprint for discrimination of microbial species based on lensless imaging of microcolonies.” PloS one 12.4(2017 ): e0174723] were able to detect microcolonies with diameters in 10-500 μm range.

An important factor which may limit the detection of microcolonies is the size and density of surface artefacts (the background) that are observable via microscopy after contacting the treated sample with the solid phase growth medium, where such surface artefacts are not representative of microbial cells or microbial cell colonies. Two example types of such surface artefacts that contribute to a background include (i) surface inhomogeneities of the gel surface and (i) lysis debris particles that remain after the lysis of the sample, such as lysis debris particles resulting from the digestion of blood cells, which persist in the sample after centrifugal washing.

The surface density of the first type of artefact, namely gel surface inhomogeneities, can be reduced through controlled fabrication of the gel. An example non-limiting method for preparing gels with a low density of surface inhomogeneities is described Example 6 below.

The size and amount of the second type of the artefacts, namely lysis debris particles may vary from one whole blood sample to another and has been found to depend on the composition of the blood lysis reagent (BLR).

International Patent Application No. PCT/CA2013/000992, titled “APPARATUS AND METHOD FOR EXTRACTING MICROBIAL CELLS” discloses a number of different blood lysis reagent compositions that may be employed for the digestion of blood components prior to centrifugation. As noted above, the presence of the blood lysis reagent causes the selective lysis of blood cells. In one example implementation taught by International Patent Application No. PCT/CA2013/000992, the blood lysis reagent may be an aqueous liquid including saponin and sodium polyanetholesulfonate (a sodium salt of polyanetholesulfonic acid, known as SPS), and a blood lysis reagent having such a composition is henceforth referred to as a “type 1” blood lysis reagent. The blood lysis reagent may also include an antifoaming agent, such as poly (propylene glycol) (PPG, e.g. with a molecular weight of approximately 2000). International Patent Application No. PCT/CA 2013/000992 teaches example concentration ranges of saponin and SPS for a type 1 blood lysis reagent, upon mixing whole blood and the blood lysis reagent, of approximately 1.5 to 80 mg/mL and 0.5 to 20 mg/mL, respectively.

As taught in International Patent Application No. PCT/CA2013/000992, SPS is an anti-coagulant and anti-phagocytosis agent and is known to inhibit antimicrobial agents (Sullivan, N. M., Sutter, V. L., & Finegold, S. M. (1975). Practical aerobic membrane filtration blood culture technique: development of procedure. Journal of clinical microbiology, 1(1), 30-36). The mechanism by which SPS assists in blood cell lysis is not well understood. Without intended to be limited by theory, it is believed that SPS may offer some level of protection to the microorganisms during blood cell lysis, reduce the incidence of entrapment of bacteria in cell debris, and/or reduce the quantity of lysis debris components that may otherwise be present in the sediment.

In another example implementation of a blood lysis reagent composition taught by International Patent Application No. PCT/CA2013/000992, a blood lysis reagent may be an aqueous liquid including Triton X-100 and SPS in a buffer having a pH ranging from 9 to 11, and a blood lysis reagent having such a composition is henceforth referred to as a “type 2” blood lysis reagent. The blood lysis reagent may also include an antifoaming agent, such as poly (propylene glycol) (PPG, e.g. with a molecular weight of approximately 2000). International Patent Application No. PCT/CA2013/000992 teaches example concentration ranges of Triton X-100 and SPS for a type 2 blood lysis reagent, upon mixing whole blood and the blood lysis reagent, of approximately 0.5 to 1.5% w/v and 5 to 10 mg/mL, respectively.

As noted above, the type 1 blood lysis reagent composition described above was found to be suitable for manual and semi-automated separation and concentration of microbial cells from whole blood as per the teachings of International Patent Application No. PCT/CA2013/000992. However, when adapting the reagent formulations disclosed in International Patent Application No. PCT/CA2013/000992 to the automated separation and concentration, and subsequent identification, of microbial cells from whole blood as per the automated methods of International Patent Application No. PCT/CA2015/050449, titled “Apparatus, System and Method for Performing Automated Centrifugal Separation”, filed on May 19, 2015, which is hereby incorporated by reference in its entirety, the present inventors found that the type 1 blood lysis reagent composition was most suitable for cases in which the quantity of whole blood was less than approximately 1 ml.

Another example blood lysis reagent for achieving a low surface density of lysis debris particles is described in International Patent Application No. PCT/CA2019/050716, titled METHODS AND COMPOSITIONS FOR THE SELECTIVE LYSIS OF BLOOD CELLS AND SEPARATION OF MICROBIAL CELLS”, and filed on May 24, 2019, which is incorporated herein by reference in its entity, and which describes example blood lysis reagent compositions and method for preserving microbial cell viability and lowering the sample viscosity to the levels that the fluidic movement operations through narrow channels on the cartridge could be performed without intolerable impediment. This blood lysis reagent composition, henceforth referred to as a type 3 blood lysis reagent, may be provided containing saponin, SPS, an alkaline buffer and optionally a non-ionic surfactant.

In one example embodiment, the type 3 blood lysis reagent may have a composition such that after the blood lysis reagent is mixed with the sample, the concentration of saponin lies between 3 and 60 mg/ml, the concentration of SPS lies between 1.5 and 50 mg/ml, the concentration of non-ionic surfactant lies within 0-3% w/v and the pH lies within a range of 7.8-10. In some example embodiments, the buffer concentration may be selected such that the effective buffer concentration lies in the range of 10-300 mM. It will be understood that a suitable concentration range for a given components of a blood lysis reagent can be determined, for a given set of conditions, by experimentally investigating the effect of changes in concentration of the given component on one or more performance metrics, such as, but not limited to, blood lysis efficiency, quantity of residual blood cell debris, microbial cell intactness and microbial cell viability. A type 3 blood lysis reagent may be provided as two or more reagents that can be stored separately and mixed prior to use, such that the saponin component of the blood lysis reagent is stored in an acidic environment that is separated from the alkaline component of the blood lysis reagent. In one example implementation, one or more of the reagents that are mixed to form the final blood lysis reagent may be stored in a solid phase.

Accordingly, in some example implementations, a sample may be processed by a type 3 lysis reagent comprising saponin, sodium polyanethole sulfonate (SPS), a non-ionic surfactant (such as, but not limited to, Triton™ X-100), a buffer (e.g. a carbonate-bicarbonate buffer), with an alkaline pH. The blood lysis reagent may also include an antifoaming agent such as SE-15 (e.g. a 10% emulsion w/v of active silicone polymer and non-ionic emulsifiers).

A blood lysis reagent without a non-ionic surfactant and a carbonate-bicarbonate buffer is known to be benign to microbial cells. However, as illustrated below, in the case of processing whole blood samples, the size of the blood debris transferred to the final cell suspension according to the use of such a blood lysis reagent has been found to give rise to an elevated level of surface artefacts (background) that can impede microcolony detection and characterization. In contrast, addition of moderate amounts of non-ionic surfactant (e.g. Triton™ X-100) and carbonate-bicarbonate buffer, along with saponin and SPS, while not significantly impacting the cell viability, can be beneficial in significantly reducing the surface artefacts arising from lysis debris.

In some example implementations, a whole blood sample with a volume up to 10 mL may be mixed with a type-3 blood lysis reagent such that the concentration of saponin in the final mixture ranges between 10-30 mg/ml (or, in some example implementations, 3-60 mg/ml), the concentration of SPS in the final mixture may range between 5-50 mg/ml (or, in some example implementations, 1.5-50 mg/ml), the effective buffer concentration lies in the range of 10-300 mM, the concentration of non-ionic surfactant lies within 0-3% w/v (or, in some example implementations, 0-1% w/v), the pH of the final mixture may range between 7.8-10 (or, in some example implementations, 8.2-9.5), and the concentration of the antifoaming agent emulsion lies within 0.005 to 0.5% (v/w).

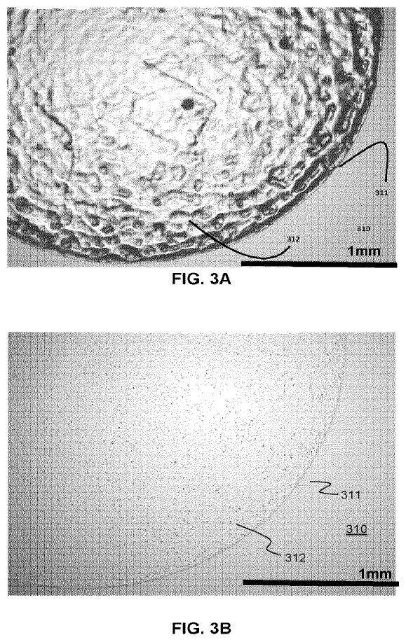

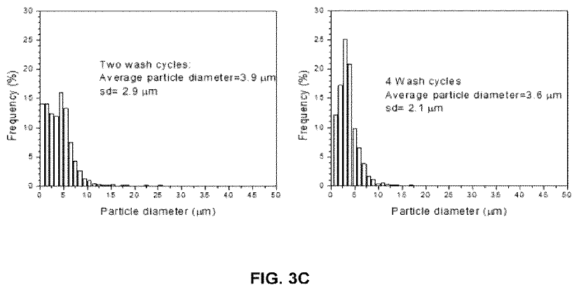

In order to illustrate the dependence of the surface artefact density on the composition of the blood lysis reagent, 4 mL of whole blood sample was processed according to the method of Example 5, both with 2 washing cycles using a blood lysis reagent having a composition as the following: 35 mg/mL saponin, 20 mg/mL SPS (BLR 1) and (ii) 35 mg/mL saponin, 20 mg/mL SPS, 0.3% w/v Triton™ X-100, and 50 mM carbonate-bicarbonate buffer, with a pH of 10 (BLR2 ). After exposure of the sample to the respective blood lysis reagents and centrifugal separation, 1 μL of each final microbial cell suspension were pipetted on a spot-on agar gel plate. The microbial cell suspension samples spread to circular areas with diameters of about 5 mm and air dried in about three minutes. These areas, which herein are labeled as mini-culture region (MCR) were imaged by a microscope equipped to a 5× objective and was presented in FIGS. 3A and 3B. In these figures the MCR, unused agar plate surface and the boundary between two regions are respectively indicated by 310, 312, and 311. As it is observed the inclusion of Triton X-100 and carbonate-bicarbonate buffer significantly reduces the background (density of surface artefacts). The present inventors found that this background level could not be significantly reduced further by increasing washing cycles. For example, this was demonstrated by treating a 4 mL whole blood sample using BLR2 having a formulation as described above with 2 or 4 subsequent centrifugal wash cycles. A 1 μL of the resulting microbial cell suspension was dispensed on the agar plate and allowed to spread and air dry. The pictures of the resulting MCRs were recorded with a 10× microscopic objective, and were analyzed for the size distribution of debris at the end of 2 and 4 wash cycles. The particles were located via intensity-based adaptive auto-threshold methods and were fitted with ellipses. More precisely, image segmentation was performed and a label was assigned to every connected group of pixels in an image such that pixels with the same label share certain intensity characteristics. The histogram of the measured major particle diameter (major axis of a fitted ellipse) distribution is presented in FIG. 3C. As it is observed, the distribution plots are qualitatively similar, despite the fact that the sample is diluted by a factor of 400-fold between 2 and 4 washes.

These results suggest that the size of the debris particles is strongly influenced by the composition of the blood lysis reagent when processing blood samples for subsequent direct colony growth. However, the requirement for the microbial cell viability, particularly in the case of Gram-negative bacteria, limits the ability to achieve smaller debris size by increasing the digestion capacity of the blood lysis reagent. This limitation can be tolerated as long as the sizes and the density of the debris does not impede the detection of microbial cell growth at microcolony level. One example criterion in this respect is that after spreading the final cell suspension on the cell growth chamber the lysis debris artefacts should not cover the surface of the solid phase growth medium with an areal fraction exceeding 90%, or preferably not exceeding 50%, or more preferably not exceeding 20%. The present inventors have found that this criterion can be satisfied by processing whole blood samples with a type-3 blood lysis reagent.

Provided that the surface density of artefacts is not prohibitively high, it is feasible to differentiate the background from microcolonies by recording multiple pictures over a time period via time-lapse imaging, or, for example, removing the background by size thresholding. In order to perform size selection, an image of the gel surface can be taken from a limited area of the surface before or slightly after incubation. In one example implementation, image analysis is performed to determine the average size of the debris particles, R_{back.av}, and the standard deviation of the debris particle size, sd, and a size threshold may be determined based on R_{back.av} and sd. The size selection criterion can be set, for example, as R>R_{threshold}=R_{back.av}+n*sd, where n is typically an integer selected in the range 1 to 6, and more typically n=3. In another embodiment, R_{back.av} and sd may be predetermined (e.g. and provided along with the cartridge or embedded in software of the device/instrument) to become available at the colony analysis stage.

In some example embodiments, the colony growth module is configured for removal of a liquid component of the microbial suspension (i.e. at least a portion of the liquid component) after flowing the cell suspension into the growth chamber, such that at least a portion of the microbial cells within the suspension are retained on the surface of the solid phase growth media. The removal of a liquid component of the cell suspension assists with the seeding of the microbial cells onto the solid phase growth medium, promoting subsequent colony growth. Furthermore, by removing a liquid component of the cell suspension, other complications associated with the presence of residual liquid are also mitigated, such as, for example, condensation and excess moisture which can interfere with the monitoring of growing colonies by optical or other (e.g. electrical) means.

In some embodiments the removal of a liquid component of the cell suspension may be achieved by evaporation, either passively via vents or vapor permeable membranes or actively by means of forced convective evaporation. In other embodiments a liquid component may be absorbed by the growth media or a growth media infused substrate.

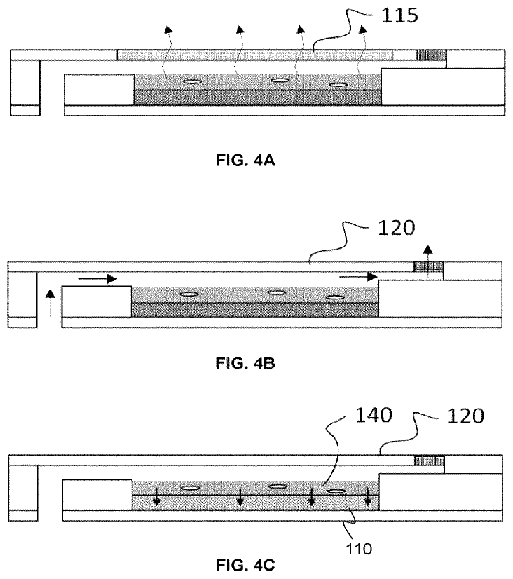

In one example implementation, a liquid component of the cell suspension residing within the growth chamber may be passively evaporated via a vapor permeable membrane 115 within the upper wall of the growth chamber as shown in FIG. 4A. In another example embodiment, evaporation may be actively enhanced by reducing the ambient pressure of the chamber such that evaporation of the cell suspension fluid will be accelerated. For instance, at 25° C., the vapor pressure of water is approximately 24 mmHg. This may be achieved by closing the chamber inlets and outlets by valves or other means and applying the negative pressure via a permeable membrane 115 in the upper wall of the chamber, or with the application of the negative pressure via a fluidic conduit connecting a closed and non-permeable chamber to a vacuum pump.

In another example implementation illustrated in FIG. 4B, air may be flowed over the cell suspension to aid in the evaporation of the cell suspension fluid by forced convective evaporation. The air may be desiccated and/or warmed to further increase the rate of evaporation.

In other example embodiments, a liquid component of the cell suspension may be removed passively by absorption as illustrated in FIG. 4C. In this embodiment the growth media is provided in one of a number of dry formats which absorbs the liquid and brings the microbial cells into contact with the growth media to promote colony growth and, in some embodiments, to immobilize the colonies for subsequent harvesting.

Various examples of suitable liquid-absorbing solid phase growth media, and methods of fabrication thereof, are described in further detail below. In some example embodiments, at least an upper portion of the growth media 110 (FIG. 2B) may have a porous network with hydrophilic properties, where the porous network is provided in an at least partially dry state, such that the internal porous network is at least partially open and capable of absorbing a liquid. In other words, the solid phase growth media 110 may be provided in a state that is not fully saturated with liquid. A liquid component of the cell suspension that is provided by the cell separation module may thus absorbed by the solid phase growth media 110 upon contact therewith, hydrating the solid phase growth media 110 and drawing the microbial cells of the cell suspension onto the surface of the solid phase growth media. The porosity of the solid phase growth media may be selected to prevent microbial cells from entering into the porous network, such that the microbial cells are retained on or near (proximal to) a surface of the solid phase growth media when a liquid component of the cell suspension is absorbed.

The use of a liquid-absorbing solid phase growth media (a solid phase growth media that is not fully hydrated and has a capacity for further absorption) solves a significant problem in facilitating the controlled adsorption, within a closed integrated fluidic device, of microbial cells from a cell suspension onto a surface for colony growth. By rapidly and efficiently absorbing a liquid component of the cell suspension, complications associated with the presence of residual liquid (e.g. residual droplets or locally accumulated pooling) are avoided. The present inventors have found that such residual liquid can hinder cell-surface association. Moreover, residual liquid can result in condensation and excess moisture, which can interfere with the monitoring of growing colonies by optical or other (e.g. electrical) modalities. Various examples of suitable liquid-absorbing solid phase growth media, and methods of fabrication thereof, are described in further detail below.

In some example embodiments, the solid phase growth media may be provided in a dehydrated or partially-dehydrated form. Thus, a liquid component of the cell suspension, upon being introduced into the growth chamber, is wicked away into the pores of the solid phase and the microbial cells are retained on the surface. One example of a dehydrated solid phase growth medium is disclosed in U.S. Pat. No. 4,565,783. A self-supporting water-proof substrate, such as polyester film, is coated with an adhesive, such as copolymer of isooctylacrylate/acrylamide (in a mole ratio of 94/6), which is non-inhibitory to the growth of microorganisms. A cold-water-soluble gelling agent, such as guar gum, along with nutrients for growing microbial cells, is dispersed into the adhesive. Upon contact with a liquid, the liquid reacts with the particles of the gelling agent and a solid phase is formed for supporting the growth of microbial cells.

In another implementation, a gellated solid phase is partially dehydrated in a low humidity environment and is maintained partially dehydrated in a humidity-controlled package until use. Alternatively, the gellated solid phase can be partially dehydrated by freeze-drying techniques.

In another example embodiment, at least a portion of the liquid component of the cell suspension may be removed via centrifugal processing of a gel-based solid phase growth medium prior to contact with the cell suspension. An example implementation of this embodiment is illustrated in FIGS. 5A-5D. The solid phase growth medium is subjected to a centrifugal force 103 due to rotation around an axis, as shown in FIG. 5A. While the figure shows the centrifugal force as being directed perpendicular to the gel surface in FIG. 5A, it will be understood that in other example implementations, the centrifugal force may be directed at an angle relative to the surface of the solid phase growth medium (e.g. within an angle of 30 degrees from a perpendicular direction). As a consequence of the centrifugal force, the gel is partly dehydrated (“dewatered”), removing a liquid component of the gel without significantly collapsing the gel, at least in the upper portion of the gel, as illustrated in FIG. 5B.

When a microbial cell suspension containing microbial cells is dispensed on (contacted with) the gel it spreads over the gel surface, as shown in FIG. 5C, with at least a portion of the liquid content of the microbial cell suspension being absorbed by the partially dehydrated gel, as shown in FIG. 5D. The microbial cell suspension can be spread over the gel, and the liquid component of the microbial suspension may be absorbed passively (for example, under gravity) or for example, under the application of a subsequent centrifugal force. After removal of the liquid component of the microbial cell suspension, leaving behind the microbial cells on the gel surface. The exuded liquid component of the gel (e.g. water) may be collected inside empty chambers behind the gel. The water exuded from the gel may be drained during centrifugation or afterward.

The dewatering phenomenon, also known as syneresis, occurs with most hydrogel materials when the elastic pressure of the gel network, formed by the helical coils of agarose polysaccharide, exceeds the osmotic pressure of water that normally cause gel expansion. External mechanical pressure such as centrifugation promotes syneresis by squeezing the gel, analogous to squeezing water out from a wet sponge. The removal of some water from the gel presents opportunities for the rapid absorption of the cell suspension by opening vacancies in the gel network to allow for new water to enter. Controlling the volume of water that is exchanged will be a key design feature of the growth chamber design. Thus, in the following section of the present disclosure, experimental observations are presented demonstrating the quantification of the dewatering phenomenon as a function of gel composition.

To simulate the water loss from the agar gel upon centrifugation, gels were placed on top of a porous membrane inside a centrifuge tube and spun at 4000 RPM. The mass of the gels before and after the centrifugation was measured, with the difference being attributed to the water loss as a result of the centrifugal force upon the gel. In one experiment, approximately 0.15 gram sections of agar gel were sliced out of Petri dish and placed into a NanoSep Tube with a 0.45 μm modified nylon filter membrane. The tube was then spun at 4000 RPM (3200 g) for 8 minutes resulting in water being separated from the gel, passing through the membrane and collecting at the bottom of the tube. The mass of gel remaining on the membrane was then measured and the percent water loss was determined according the mass difference of the gel before and after centrifugation.

The results, which are presented in FIG. 5E, show that agar gels made with a higher content of agar (1 to 3%) are more resistant to dehydration. This greater retention of water is due to the greater strength of the gel, making it more resistant to syneresis from the centrifugal force. Stiffer gels have also been observed by the present inventors to be less prone to cracking or deformations during the centrifugation. Different gel additives were also investigated to determine their effect on water loss by centrifugation. The additives chosen were mainly other polysaccharide hydrocolloids that had either gelling (agar, gellan gum, gum Arabic and carrageenan) or non-gelling (dextran) properties. In addition, gellan gum (Gelzan™ CM) and carrageenan are often used as agar substitutes for microbial cultures. It was shown that 1.35% agar with 0.5% gellan gum retained the most water after centrifugation in the 0.45 μm Nanosep tube (average of 27% water loss by mass) compared to 1.35% (49% loss) or even 1.85% agar alone (39% loss). It is believed that this greater retention is due to the greater strength of the gel which resists the external pressure from the centrifuge.

The experiment was repeated using a filter membrane with a smaller pore size. In this case, the gels were prepared in the autoclave and while still warm (~50° C.), dispensed over a 10,000 MWCO (<5 nm pore size) cellulose membrane of an Amicon Ultra-15 ultrafiltration tube. Each tube had about 1.5 grams of gel when cooled, which was then centrifuged for 9 minutes at 4000 RPM. The mass of the gel remaining on top of the membrane was then measured and the percent of water loss was calculated and presented in FIG. 5F. The results reveal how a smaller membrane pore size reduces the dewatering level for all gels compared to the larger 0.45 μm pores. The addition of 0.5% gellan gum to 1.35% agar leads to less water loss at about 16% (that is, greater water retention) compared to the other gels which were between 22 to 23%.

The presented inventors observed that dewatering can be reduced or prevented if the gel is well confined at its side and back surfaces. The use of gel confinement can be employed to limit the dewatering level (the level below which dewatering is eliminated or reduced) such that the volume of liquid removed from the gel corresponds to the volume of the liquid component of the sample which is desired to be spread on the gel surface and absorbed by the gel via the rehydration process. Without being bound to theory, it is hypothesized that dewatering leaves voids in the molecular coils within the gels which could potentially be refilled with fresh liquid (e.g. an aqueous solution) from a microbial suspension. To illustrate this experimentally, the portion of gels that were dehydrated after centrifugation in the 10,000 MWCO ultrafiltration tube (see above) were removed from the tube and placed on a small plastic dish to determine their mass. Each dish was filled with 2 mL of fresh water to soak into the gel. After 20 minutes of soaking, the excess water was removed by pouring it out of dish and carefully wiping away the remaining drops from the dish and the gels. The mass of the gels was then obtained and recorded, as shown in FIG. 5G, as the percent mass gain with respect to the mass of the dehydrated gel.

The results suggest that the amount of water that was lost during centrifugation can be regained by rehydrating the gel. That is, the percent of dehydration is similar to the percent of rehydration of each gel. Therefore, the voids in the gel network that resulted when water was expelled from the gel are refilled with approximately the same volume of water when the gel is rehydrated.

Partial dehydration of gels can also be achieved through water exchange with the environment. If the dehydration level of the gel is relatively low, the process is reversible and nearly full rehydration can be achieved. This property is useful if the centrifugation-assisted dewatering of the gel would be prevented by near-complete confinement of the gel through appropriate chamber design, while the rehydration for the purpose of sample seeding is performed by centrifugation. In order to illustrate the feasibility of this approach, the following experiments were performed.

Agar gels with agar concentration of 1, 2, 3 and 4% wt., were dehydrated during open storage at 4° C. for 3 days in 35 mm Petri dishes. The mass of the gels in the dishes before and after the storage was recorded, demonstrating that each gel lost on average 6 to 7% of mass by water (see FIG. 5H). The gels were then rehydrated by adding 1 mL of water to the Petri dishes on top of the gels, and after 4 hours, the water was removed and the gels were reweighed, showing that 1% wt. agar gel regained the same amount of water that it had lost to dehydration, while gels with 2% wt. or more gained approximately 1% in mass after the 4 hours of rehydration. To determine if the water capacity can exceed its original amount prior to the first dehydration, the gels were soaked for an additional 24 hours in 1 mL of water. After the water was removed and the masses determined, it was found that with 1% and 2% wt. agar gels no extra water was absorbed beyond that which was absorbed after the initial rehydration. Gels made with 3% and 4% agar increased their mass by 2.1 to 2.6% from their original mass through additional water absorption.

Without being bound by theory, the results presented in FIG. 5H may be considered by considering the agar gels' complex helical coils made of agarose polysaccharides surrounded by water (agar gel also contains agaropectin which is a nongelling polysaccharide that makes up to 30% of agar's composition by weight).

Some of the water is bound to the agarose coils (and possibly the agaropectin) via hydrogen bonding interactions, but most of it exists in an unbound, fluid-like form that does not provide structural support to the gel coils. With the agar gels made with 1to 4% agar, all gels become dehydrated by the same extent (6 to 7%) by losing the unbound water by evaporation. Upon rehydration, water re-enters the gel driven by an osmotic gradient and re-occupies some of the voids left behind by after evaporation. With gels made with higher concentration of agar (>2% wt.) the osmotic gradient is slightly greater, leading to the uptake of more water than the initial amount in the fresh gel (2-3% more), but the rigid helical structure of the gel limits its volume due to limitations on where extra water can enter.

In order to determine the rehydration in the presence of gravity alone, two gels made with 2% wt. agar concentration in 35 mm Petri dishes were dehydrated to varying extends; one was 30% dehydrated after storage unsealed at room temperature, the other was approximately 2% dehydrated after sealed storage at 4° C. 4 regions of 25 μL of a red dye solution were then added to each gel (for improved visualization) and the time taken for the solution to completely penetrate the gel was observed. In the case of the 30% dehydrated gel, the solution took approximately 5 minutes to soak inside the gel, leaving a red spot on its surface. In the case of the 4% dehydrated gel, the solution took close to 20 minutes to soak through the gel.

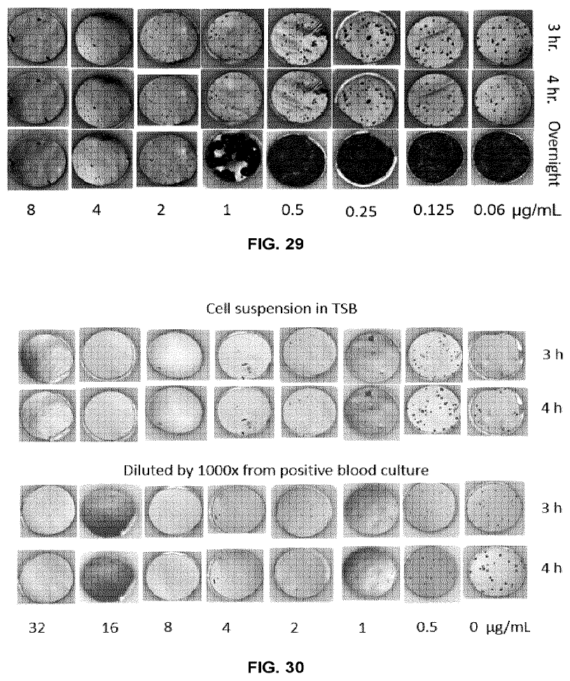

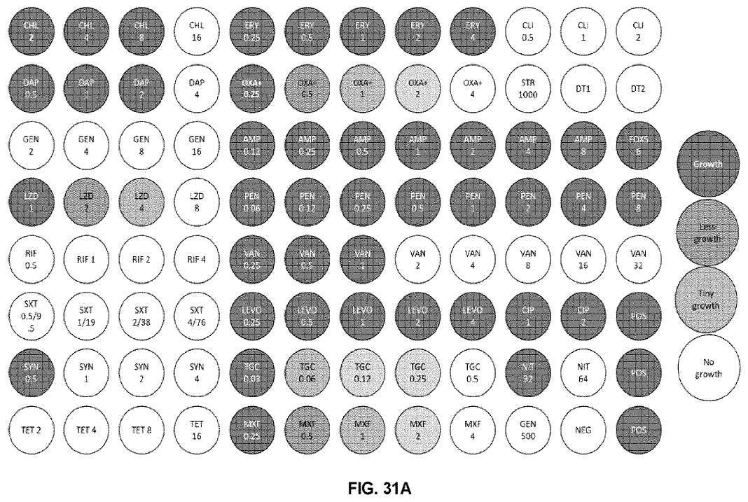

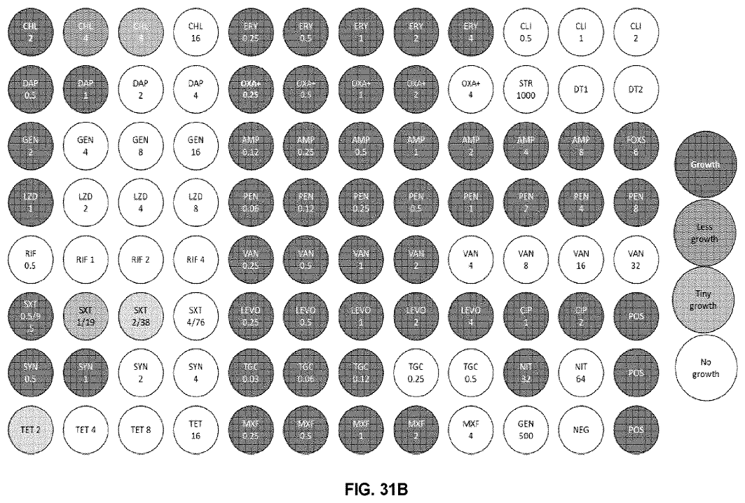

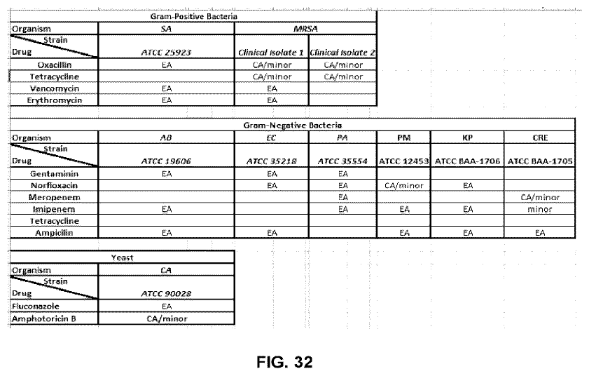

In one embodiment, presented in FIG. 51, the gel is placed on a membrane 131 that resides over an absorbent material (e.g. an absorbent pad) 132. In some example implementations, the membrane may have pore sizes ranging from 0.2 μm to less than 5 nm (eg. size exclusion of 10,000 Daltons). In such cases, water from the gel only passes through the membrane when a sufficient pressure (e.g. >30 psi) is applied to the gel by centrifugation. Examples of suitable membrane materials include polycarbonate, polyester, PTFE, PEEK or PVDF of thicknesses ranging from 50 to 250 μm. A glue, or other adhesive may be employed to bind the membrane to the side walls of the growth chamber in order to prevent hot liquid gel from leaking onto the absorbent material while the gel is being poured into the dish. The absorbent pad is placed at the bottom of the dish and absorbs the water that is removed from the gel upon centrifugation and passes through the membrane on top. The absorbent material may be made from materials such as, but not limited to, cellulose or glass fibers. During centrifugation, the exuded water from the gel is displaced and absorbed by the absorbent material. In another embodiment that is shown in FIG. 5J, channels are provided beside gel for draining the exuded water.

FIG. 5K illustrates an example growth chamber employed to investigate an example implementation of the embodiment illustrated in FIG. 5I. A standard 35 mm clear, polystyrene Petri dish was lined with a 2-sided adhesive ring 133 around the edge of the bottom and two layers of Whatman No. 2 paper 132 were placed inside the ring. The paper was used as an absorbent material for absorbing the water released during dewatering process. 0.5 μL of a dye solution was spotted in multiple locations on the absorbent paper, as indicators spots, as shown at 134, in order to estimate the amount of water that was transferred from the gel to the absorbing paper during storage.

It is desired that the membrane 131 prevents water transfer under storage condition and only allows the transfer during centrifugation. In this regard, the suitability of two different membranes was tested: a polycarbonate membrane with a pore size of 0.1 μm and a PTFE membrane with a pore size of 10 μm. The PTFE membrane is strongly hydrophobic and is expected to outperform the polycarbonate membrane in preventing water transfer prior to centrifugation. In order to investigate this hypothesis, two example growth chambers were prepared: one growth chamber having a polycarbonate membrane and the another growth chamber having a PTFE membrane. Agar solution (1.35% wt.) at 50° C. was then poured on top of the each membrane and allowed to set into a gel and solidify over 3 hours. Each chamber was sealed with parafilm and was stored in 4° C. for overnight. Photographs of the gels were taken at 7 hours and 20 hours after pouring the gel and were compared with photographs taken at three hours, as presented in FIG. 5L. As it is observed, despite having much larger pore sizes, the PTFE membrane nearly prevents water transfer under the storage condition mentioned above, with only minimal blurring of the dye spots being observable. Accordingly, it is expected that a PTFE membrane with even smaller pore sizes can prevent water transfer more effectively.

A second aim of the investigation of the example growth chamber of FIG. 5K was to illustrate that the PTFE membrane allows the transfer of the gel water to the absorbent material during centrifugation, enabling automated sample seeding. To this end, three growth chambers were prepared: one growth chamber being constructed with a membrane filter according to the embodiment of FIG. 5K, and the other two growth chambers being constructed without a membrane filter and absorbing paper. The growth chamber having the membrane and one of the non-membrane growth chambers were centrifuged for 8 minutes at 3200 g. After centrifugation, 100 μL of dye solution was dispensed on four spots of each gel (including the gel of the growth chamber that was not subjected to centrifugation) and allowed to settle for 5 minutes to assess the capability of each gel to be locally rehydrated via absorption of the dye solution.

The photos of the gels after dispensing of the dye solution are shown in FIG. 5M. As it is observed, for the chamber that was not subjected to centrifugation, the dye solution persists as drops on the gel surface. In contrast, in the case of the chamber without the membrane and absorbent pad, during the centrifugation phase, a small fraction of the water content of the gel was removed through the dewatering process (~100 μL), via leakage through the sides to the gel surface. This released water had been drained before dispensing the dye solution, but due to detaching of the gel from the chamber surface, a portion of the dispensed liquid flowed to the gap between the gel and the chamber wall. In contrast, the growth chamber with the membrane and absorbent pad absorbed the dispensed sample very easily. This absorption process could be accelerated by centrifuging the growth chamber after dispensing of the dye solution, for example, at speeds of at least 1000 rpm for a time duration of at least one minute.

In some example implementations involving centrifugal dehydration of the gel-based solid phase growth medium, the centrifugal force may be applied to the gel during a centrifugal separation process (e.g. a separation process performed using the separation module of the integrated fluidic device).

In one example embodiment, the cell suspension may be contacted with the solid phase growth medium and centrifugation may be subsequently employed to simultaneously remove a portion of the liquid component of the gel and introduce, into the gel, the liquid component from the microbial cell suspension.

The volume of the liquid that is exuded from the gel is preferably similar (e.g. within 25%, within 10% or within 5%) to the volume of the cell suspension. It will be understood that the ability to perform partial centrifugal dehydration of the gel and absorption of the liquid component of the microbial cell suspension depends on the factors including, but not limited to, gel concentration, gel thickness, gel surface area, and duration and magnitude of centrifugal forces experienced by the gel. As suitable set of parameters may be determined empirically through experimentation in order to achieve a suitable performance, such as selecting the material and processing parameters such that the volume of the liquid exuded from the gel is approximately equal to the volume of the microbial cell suspension.

For example, in the example case of cell separation by the example cartridge shown of FIGS. 13A-13D, the volume of the final cell suspension is 100 μL. In such an example case, the gel included in the growth chamber may be provided with a surface area of 10 cm² and a thickness of 5 mm. For the example case of 1.5% agarose gel, the volume of the exuded water over about 10 minutes of centrifugation at 4000 g is close to 100 μL. The present inventors have found that more concentrated gels, such as 4%, exudes about 20 μL of water under similar conditions, and the skilled artisan may employ the preceding experimental method, or variations thereof, to determine the volume of the liquid component of the gel that is extruded under a given set of material and processing conditions and select a suitable corresponding volume of the microbial cell suspension.

As noted above, the cell seeding process (i.e. the process of contacting a microbial cell suspension with the solid phase growth medium and allowing its liquid content to be absorbed by the partially dewatered gel), can be performed either by passive absorption (e.g. assisted by gravity) or centrifugally. The present inventors have found that passive seeding (by mere contact of the cell suspension with the gel surface) of the aforementioned gel (1.5% gel with 5 mm thickness and 10 cm² surface area and 100 μL cell suspension volume) may take up to 20 minutes at room temperature. This time can be reduced by employing centrifugal seeding. For example, after contacting the microbial cell suspension with the dewatered gel, the growth module may be centrifuged by ramping up to 500-4000 g and subsequently ramping down, with the centrifugal force directing the microbial cells in the suspension toward the gel surface. This action may take between 30 s to 1 minute. When using a lower centrifugal force, for instance between 500-1500 g, dwelling for between ramp up and ramp down (e.g. for 20-40 seconds) may provide more efficient cell seeding.

After the microbial cells from the cell suspension are contacted with the surface of the solid phase growth media while maintaining at least the growth chamber in a closed state, the integrated fluidic cartridge may be incubated, with at least the growth chamber in the closed state, in an environment with a temperature suitable for promoting microbial cell growth (e.g. 37° C.) and colony formation.

In one example embodiment, a portion of the integrated fluidic cartridge containing at least the growth module (e.g. only the growth module) may be detachable from the remainder of the integrated fluidic cartridge. This embodiment is advantageous in two respects. Firstly, the incubation of the remainder of the integrated fluidic cartridge, which may include biological waste, is avoided. Secondly, the detached portion of the integrated fluidic cartridge containing the growth chamber can be beneficial when the integrated fluidic cartridge is incubated in an incubator equipped with colony monitoring modalities which employ light transmission through the growth medium.

Once the microcolonies are formed, they may be employed for subsequent testing. For example, in one example embodiment, the upper wall can be removed or opened to provide access to the colonies for the harvesting thereof (e.g. removal and transfer) for subsequent processing such as, but not limited to, MALDI identification assays, metabolic identification assays, and AST. The upper wall 120 may contain a removable lid, a peelable section or other means of opening to facilitate access to the colonies.

The present example embodiment involving the contact and incubation of separated microbial cells with a solid phase growth media may be advantageous for a number of reasons. Firstly, as noted above, the initial separation step can be effective in reducing the concentration of antibiotics that may already be present in the sample. Indeed, in the case of whole blood samples that are obtained from patients suspected of sepsis, it is common for empiric antimicrobial therapy to be initiated prior to initial phlebotomy. Secondly, in bypassing the conventional liquid phase culture step, the present example method facilitates the direct formation of microbial colonies from a previously uncultured sample, saving a considerable amount of time (e.g. 1-2 days) and consequently reducing the time for positivity.

A third benefit of the present example method is that the spatially distinct growth of individual microbial cells on the solid phase growth media facilitates the independent growth of different cell classes (i.e. cell types; microbial cells of different taxa, such as different genus, species, or strain, or bacterial vs. fungal cells) from a polymicrobial sample. Accordingly, the present example methods permit the direct determination of the inherent and true polymicrobial nature of the sample, unperturbed and unbiased by competition that would otherwise occur in a liquid culture environment. As a result, one may identify, based on one or more properties of the colonies grown in the solid phase, the presence of two or more distinct microbial cell classes, with the ability to perform further processing (e.g. identification, such as via MALDI, or AST, such as via broth microdilution or other approaches), in a separate and independent manner for two or more of the different cell classes present. In some example embodiments, a single colony associated with a given cell type may be processed, while in other example embodiments, two or more colonies from a given cell type may be pooled and processed.

The growth of colonies formed on the solid phase growth media, following the distribution of the separated microbial cell suspension thereon, may be monitored according to one or more detection modalities. In one example implementation, optical detection may be employed to monitor the growth of one or more microbial cell colonies. For example, in one example implementation, a camera may be employed to image at least a portion of the solid phase growth media. In another example implementation, an array of photodetectors may be employed in the absence of an imaging element to obtain an image of one or more microbial cell colonies, where a growth surface associated with the colonies is located in sufficient proximity to the array of photodetectors to form an image thereon upon illumination thereof. In implementations in which the field of view of the optical system is less than the spatial extent of the solid phase growth media, the optical system may be scanned relative to the solid phase growth media, or vice versa, in order to facilitate the optical interrogation of the entire solid phase growth media surface, or a desired subset thereof.

An image may be processed using known image processing algorithms to identify microcolonies and to optionally estimate one or more dimensional measures of an identified microcolony. For example, publicly available software, such as the ImageJ/Fiji program, may be employed. In one example method, after converting an image into a greyscale image, the greyscale image may be binarized by applying local adaptive image thresholding according to Phansalkar method, based on histogram analysis of intensity levels. Adaptive image segmentation may then be employed according to Phansalkar method, with dimension constrains that partition an image into segments for microcolony identification. Optional further analysis of the identified segments may then be employed to determine metrics of interest associated with a microcolony (e.g. circularity, area, major axis, and minor axis). There are many different example algorithms that can be used to calculate the threshold in a bias-free manner. The Phansalkar thresholding method is a modification of Sauvola's thresholding method optimized for low contrast images [Phansalskar, N; More, S & Sabale, A et al. (2011), “Adaptive local thresholding for detection of nuclei in diversity stained cytology images.”, International Conference on Communications and Signal Processing (ICCSP): 218-220, doi:10.1109/ICCSP.2011.5739305]. Other example methods include the Bernsen, Contrast, Mean, Median, MidGrey, Niblack, Otsu and Sauvola methods.

In one example embodiment, the microbial cells of a microcolony may be interrogated (e.g. via image processing or the detection of one or more optical signals, such as a Raman signal or a fluorescence signal) to classify the microbial cells of one or more colonies that are growing on the solid phase growth media according to two or more microbial cell classes. This determination of a class may be referred to as a “presumptive identification” or “presumptive classification” when a subsequent classification modality, having either a higher confidence/accuracy or a larger set of classes, is subsequently performed. It will be understood that the class of the cells may be determined based on colony morphology or other techniques.

For example, as noted above, the solid phase growth media may be provided with chromogenic or fluorogenic substrates which change give rise to specific or non-specific staining of the colonies (e.g. detectable spectral features or signatures), as described, for example, in European Patent Application No. EP1088896A2.

Alternatively, the scattering pattern of a monochromatic light transmitting through the colonies can be used for classifying genus and species levels and some cases down to serovar levels, for example, according to the methods described in U.S. Pat. No. 8,787,633 or in International Patent Application Publication No. WO 2016/162132. For example, a presumptive identification of microbial cells within a microcolony may be performed by exposing a target microcolony to a beam of coherent light, monitoring a diffraction pattern resulting from the diffraction of the light by the microcolony, and processing the monitored diffraction pattern and a reference diffraction pattern to determine a classification measure associated with the microbial cells of the microcolony.

The determination of a classification measure, at least to a broad class such as fungal vs. bacterial and/or Gram-negative vs. Gram-positive, may be employed for the drug-bug selection (e.g. the selection of a suitable set of test antimicrobial agents based on Gram status) if a more complete species level microbial identification test is not initiated or performed prior to the antimicrobial susceptibility testing (e.g. for reasons such as time or cost saving or the scarcity of the colonies). In some example embodiments, imaging may be performed at a plurality of wavelengths in order to collect a hyperspectral image set that can be processed to assist in cell type identification and generate a presumptive identification of a class of the microbial cells.

As noted above, the present presumptive identification or presumptive classification step may result in the classification of the microbial cells, prior to cell harvesting, into a cell class such as Gram-positive bacteria, Gram-negative bacteria, fungi and optionally a subclass encompassing one or more species. The commonplace Gram stain test is an example of presumptive identification. In the case of the microcolonies non-destructive and reagent-less methods are preferred since the downstream assays such as AST require viable and uncompromised cells. Among the non-destructive and reagentless (label/marker free) methods are optical methods, including those based on fluorescence, and elastic and non-elastic scattering. Raman (micro)spectroscopy is an example of non-elastic scattering and combinations of bright field and dark field microscopy or laser diffraction from colony are belong to elastic scattering categories. Another example optical modality for obtaining at least a preliminary microbial cell class determination is Fourier transform infrared microscopy. Underlying mechanisms for differentiating between classes of pathogenic microbial cells are related to their characteristic attributes and include, but not limited to, cell wall composition, cell shape, and cell motility. This latter attribute gives rise to species specific packing of the cells across the microcolony.

In the case of employing the scattering pattern for microbial classification, for example, a convolutional neural network, or variation thereof, trained using a library of ground truth images of the growth of reference strains of microbial cells, may be employed to identify a given microbial cell colony. The library of images may include images of reference strains at various known growth times. For example, a plurality of neural networks may be separately trained using images from different points of time during colony growth of reference strains, such that at a given time during the growth of colonies from an unknown sample, a suitable neural network may be used that was trained using image data corresponding to the given time (or within a time window relative thereto). Alternatively, a single neural network may be trained using images from the different reference strains at the different time points.

In one example embodiment involving optical imaging of colonies grown on solid phase growth media, the directly or indirectly measured colony dimensions or size may be employed, optionally along with more properties of a given imaged colony (such as the type of microbial cells in a colony; e.g. genus, family, species or strain) to estimate the number of the microbial cells in the colony and/or determine when the incubation can be terminated and the colony is harvested for downstream applications, such as, but not limited to, antimicrobial susceptibility testing. This determination may be based, for example, on the minimum number of microbial cells that are required to facilitate antimicrobial susceptibility testing for a given number of bug-drug combinations, at a given number of concentrations, with a given number of controls, or, for example, a sufficient number of microbial cells to facilitate identification via MALDI. In some example embodiments, a detected colony may be harvested with a size of less than 1 mm, less than 500 microns, less than 250 microns, less than 200 microns, less than 150 microns, less than 100 microns, or less than 50 microns.

In general, the determination of a suitable growth time to achieve a desired number of microbial cells will vary depending on the cell type (e.g. genus, family, species, strain) and the downstream application. The relationship between the microbial cell type and the time to reach a sufficient cell count for subsequent testing may be established according to a lookup table. For example, an automated system may employ optical image processing to determine the class of cells (e.g. an inferred or estimated microbial species) associated with a given colony and then employ a pre-determined relationship (e.g. a lookup table or a predetermined functional relationship) to determine, for example, a suitable time at which a sufficient number of microbial cells are present in one or more colonies for subsequent processing, or, for example a suitable size measure (e.g. radius or other spatial measure) of the colony for which a sufficient number of microbial cells are present in one or more colonies for subsequent processing. In some example embodiments, multiple criteria involving both a colony size measure and time may be correlated with the microbial cell class in order to estimate when a sufficient number of microbial cells reside within a colony.

In order to illustrate an example of the dynamic nature of microcolony formation based on microbial cells obtained directly from a whole blood sample, a 4 mL whole blood sample was spiked with 3000 CFU of Proteus mirabilis (PM) cells and treated according to the method described in Example 5 below. One μL of the resulting cell suspension was dispensed on each of four agar plates and was allowed to spontaneously spread to a circular area with a diameter of ~5 mm, which is henceforth referred to as a “mini culture” region (MCR). Images of a portion of the resulting MCRs are presented in FIG. 6. By visual inspection of the images at 3 and 4 hours following the onset of incubation, some microcolonies, which are indicated by arrows, can be observed. However, in order to detect microcolonies at shorter incubation times, for example within 2 hours, the images may be analyzed for differentiating the microcolonies, marked by arrows, from the background.

One example method for microcolony monitoring is described as follows. As is observed from FIG. 7, in order to align imaging data acquired at different time points (0, 2, 3 and 4 hours after seeding, as shown in the figure), 2D-2D registration (employing translation and rotation) with rigid transformation constrains was performed. The corresponding intensity feature points between the t₀=0 hours image and each further image (t₂=2 hours, t₃=3 hours, t₄=4 hours) were automatically identified using the key-point detector SURF and used for aligning imaging date with respect to to. Intensity features present at to were classified as background while intensity features appearing on further images (cells/bugs) were classified as foreground. The position of given individual microcolonies have been marked in consecutive images.

It will be understood that a wide variety of registration methods may be employed to perform image registration, including, but not limited to, feature-based, intensity-based, and nonrigid registration algorithms. Examples of suitable feature-based algorithms include the SURF (Speeded Up Robust Features) and SIFT (Scale-invariant feature transform) methods.

In one example implementation, image registration may be performed via an intensity-based algorithm as follows. The algorithm transforms the moving image (image acquired at a later time point) so that it is spatially registered with the fixed/reference image (image acquired at a later time point). Based on the set-up, the type of transformation to perform was defined as ‘rigid’ or ‘affine’. According to the simplified definition, the algorithm internally builds a multi-resolution pyramid in memory (with a user specified pyramid level) and solve an optimization problem on each level of the pyramid. In other words, the algorithm builds an image pyramid that has N levels (e.g. N=5). At each pyramid level the image dimensions are decreased by a factor of 2. Optimization starts at the coarsest level of the pyramid and continues until either user-permitted number of iterations is reached, or until the optimizer converges attempting to refine the current transformation estimate on the following pyramid level.

The determination of the background allows for enhanced detection of microcolonies. For instance, despite the unusually large translational and rotational offsets between the 4 acquired images in the case of FIG. 7, the identification of the colonies at t₃=3 hours after incubation is unambiguous. Using this method, it becomes easier to develop fully automated microcolony identification (reduction of time to positivity TTP) and tracking system for screening the image sequences of the unstained living microorganisms. The robustness of method is illustrated below, in connection with results presented in FIGS. 9A to 9H, in comparison with estimates based on the aforementioned background thresholding method (e.g. declaring a spot a microcolony if its radius R>R_{threshold}=R_{back.av}+n*sd).

The present example time-lapse imaging method is a semi-quantitative imaging technique in which a series of images of the same scene (or approximately the same scene) are taken at different time points to capture the dynamical changes, while a static component is classified as background and can be removed. Current approaches for dynamic profiling of microcolonies rely on assumption of a static background and illumination. However, such a technique may be a subject to a number of processing variations if spectral or spatial characteristics of debris or surface of the solid growth media are not static. Firstly, it is noted that a suitable environment should be provided that permits the microcolonies to remain viable while the surface is not substantially aging (evaporation of liquid from the surface of the solid growth media associated with changes of the dimensions of the debris and its displacement) during the acquisition of the images. To address these issues, controlling the temperature and humidity, among other factors, can be beneficial for designing incubator. In one example embodiment that is presented in FIG. 15A, one or more growth modules may be placed in an incubator having a transparent and optically flat window through which the images are taken. In another embodiment, the objective and the growth modules may be placed inside an incubator with controlled humidity and temperature.

In order to characterize the performance of the present example method for the rapid and direct formation and detection of microcolonies, two characteristics of common pathogens found in blood stream infections were measured, namely (i) lag time and (ii) growth rate. The recovery fraction of these pathogens from blood samples was also measured employing the methodology of FIG. 14 and the type-3 blood lysis reagent described above.

The growth rate on the solid phase growth medium (gel) was determined following the steps of Example 7 below. The number of colonies forming units in the MCRs were counted and its logarithm (Log(CFU)) was plotted versus incubation time, as presented in FIG. 8A for the case of Proteus mirabilis (PM). The slope of this curve, i.e. 0.97, is used to calculate the growth rate, through the relation growth rate=slope/log(2)=3.23 cycles/hour. As another example, the growth rate of Staphylococcus epidermidis (SE) was measured and the measured v(Log(CFU)) versus incubation is shown in the time plot in FIG. 8B. The calculated growth rate is 2.4 cycles/hour. This growth rate is about 1.5 times higher than the measured growth rate of Staphylococcus epidermidis incubated on a CMOS chip [Jung, Jae Hee, and Jung Eun Lee. “Real-time bacterial microcolony counting using on-chip microscopy.” Scientific reports 6 (2016): 21473.]. Depending on the preparation method of the stock solution, the seeded microbial cells may pass through a lag phase before proliferation to the microcolony. For instance, as it is observed from FIG. 8C, Pseudomonas aeruginosa (PA) cells show about 2 hours of lag time. The linear trend in semi-logarithmic scale, is expected to continue while the number of cells in a colony is sufficiently low such that most of cells can divide. Once the number of cells at the inner region of the microcolony, which are deprived of space for proliferation, exceeds the number of cells at the periphery, the overall growth rate of the microcolony is expected to decrease. Such deviations have not been observed by present inventors for microcolonies containing as many as 10⁶ cells in the case of E. coli, as illustrated in FIG. 8D. Accordingly, using growth rate and lag time data to estimate the times required for reaching desired cell number appears to be justified.

FIGS. 9A-9F present the measured time lag and growth rate of seeded cells by microcolony detection for a collection of microbial cells species that constitute the majority of pathogenic microbes that are typically encountered in blood stream infections. The table also includes the measured cell recovery fraction, i.e. the fraction of cells that are successfully separated from the spiked blood sample and resuspended in a cell suspension while maintaining their viability, and are thus able to form colony, determined according to the method of Example 8. In addition, the table also presents the estimated time to positivity (TTP) for the present example growth methods involving colony growth on a solid phase growth medium (“solid phase”), as determined by the time at which the microcolony is discernable relative to the background (using the example methods disclosed above).

The TTP will be affected by the sensitivity of the detection method and its associated analysis and the background. In the simplest case of interrogating the presence of a microcolony grown from the microbial cells which have been separated from a whole blood sample by employing the simple size selection method described above, the TTP can be estimated as follows. The threshold size R_{threshold}=R_{back.av}+n*std was calculated using the parameters in the case of 2 washes in FIG. 3C: R_{threshold}=2+3*1.5=6.5 μm. Assuming the worst-case scenario of closed packing and an average bacterial size of 1 μm², the number of cells inside a circle with radius R_{threshold} will be about 120 CFU. Thus, TTP=T_{lag}+7/growth rate.

In the case of fungal species, as a consequence of their large size relative to bacteria, a single division that results in a binary division may be sufficient to detect a microcolony and arrive at a determination of positivity. This is illustrated in FIG. 10, which shows the time-lapse images of a section of a blood agar plate, on which 1 μL of a microbial cell suspension containing microbial cells separated from a whole blood sample had been dispensed. As can be calculated from growth-rate data in FIGS. 9G and 9H, after approximately 4 hours of incubation, the number of cells has increased by a factor of ~3. The proliferation of fungal cells is easily recognized comparing two photos. Thus, it is concluded that in a large distribution of fungal cells the time to positivity is TTP=T_{lag}+1/growth rate. In a typical blood sample, the number of cells in single digits and the Poisson statistics cannot be ignored. Thus, the formula should be replaced by TTP=T_{lag}+n/growth rate, where n is larger than one. In one example implementation, a value of n=2 was employed.

The characteristic growth rates are comparable with growth rate in planktonic state. In order to illustrate this concordance, the growth rate in liquid culture was estimated from experiments that were performed as described below. Ten mL samples of whole blood, spiked with different strains of microbial cells at a concentration of 5 CFU/mL, were inoculated into respective BacT/ALERT® FA Plus culture bottles and incubated in BacT/ALERT@VIRTUO. After the incubator indicated positivity, a 1 mL aliquot was drawn from each bottle, serially diluted, and plated for determining number of CFUs. Ignoring lag time, and assuming that the growth rate is constant, the growth rate was estimated based on the initial spiked concentration ratio and the final bacterial concentration at positivity by plate counting, and time to positivity (TTP). As it is observed from FIGS. 9A-9H, the growth rates on solid phase growth media and in liquid phase growth media are similar. However, as can be clearly appreciated by the TTP values, the solid phase is advantageous as a consequence of the localized nature of the microcolony, facilitating detection on the solid phase at a much earlier time. For example, while most bacterial species are easily detected in ~3 hours after plating according to the present microcolony example method, the TTP for incubation in a culture bottle is typically above 10 hours.

FIG. 9A-9G also includes estimates of the time needed for a bacterial cell to result microcolonies having 10⁴ and 10⁵ cells. These quantities are relevant for performing subsequent microbial identification and/or antimicrobial susceptibility testing, as discussed below.

Considering now the example case of performing subsequent testing on microbial cells grown in colonies using conventional methods, the number of bacterial and fungal cells required for performing microbial identification with VITEK®-MS [bioMérieux] are respectively approximately 10⁵ and 10⁴ CFU per MALDI spot. Based on experimental observations of colonies grown on solid phase growth media according to the present example methods, fast growing bacteria, such as E. faecium and E. coli, can reach the desired number of 10⁴-10⁵ cells in about 5 hours, while the slower growing bacterial cells, such P. aeruginosa, will take about 7 hours. For fungal species the incubation time to reach the desired cell number may be over 10 hours.

In some example embodiments in which optically imaging is employed for the detection of colony growth, the microbial cells from multiple colonies may be combined in order to achieve a sufficient number of microbial cells for subsequent testing (e.g. MALDI or phenotypic AST). For example, in the preceding example implementation involving the optical imaging of colony growth for the determination of when ~10⁴ CFU are available, if 10 colonies were present (and determined to be associated with a single type of microbial cell via optical image processing), then only 10³ CFU per colony would be required, thereby reducing the required time for growth by log₂10 (=3.3) doubling cycles. In general, without intending to be limited by theory, in embodiments in which microbial cells from multiple growing colonies are monitored and combined to provide a given number of microbial cells, the time duration for growth, relative to that in which a single colony is employed, is reduced by log₂N doubling cycles, where Nis the number of colonies. In polymicrobial cases in which at least some of the multiple colonies pertain to different microbial cell type, the required time to achieve a sufficient number of microbial cells (using pooled colonies) for subsequent testing may differ significantly among different cell types, both because of cell type dependent growth time and the number of colonies per cell type. This dependency may be prescribed, for example, in the form of a lookup table, or, for example, in the form of a mathematical relationship that prescribes the dependence on colony number based on cell-type-specific parameters that are stored in a lookup table.

As mentioned above, a minimum bacterial cell number or concentration range may be needed to perform antimicrobial susceptibility testing by incubating the cells with the antimicrobial agent in a liquid medium. This requirement may be more relaxed in the case of performing antimicrobial susceptibility testing on solid phase growth media, which is described in detail further below. In this latter case, the liquid content aliquot of the microbial cell suspension which is inoculated on the surface is at least partially absorbed into the gel network, leaving behind bacterial cells in close proximity of each other. Accordingly, the minimum cell content of the colony may be predetermined by the required concentration range and the volume of the liquid into which the colony is suspended.

The enumeration of the colony cell content (e.g. the determination of whether or not a sufficient colony size and/or cell count has been achieved, such as at the ~10³-10⁴ CFU level) can be achieved by different approaches. In one example implementation, one or more geometrical or optical properties associated with a colony (such as, but not limited to, radius/area or scattering/reflected/transmitted intensity, as determined from image processing methods, such as image segmentation) may be processed to determine whether or not a sufficient number of microbial cells reside within the colony for subsequent processing, based on a comparison with reference data associating the one or more geometrical properties with microbial cell count. In another example, a neural network may be employed to determine, based on the imaging of a given microbial cell colony, whether or not a sufficient number of microbial cells reside within the colony for subsequent testing, where the neural network is trained based on images of reference strains having known associated cell counts (or, for example, a known binary determination of whether or not a sufficient number of microbial cells is present in the colony for a given type of subsequent testing). In some example implementations, the determination of whether or not a sufficient number of microbial cells resides within a given colony may be determined, in part, based on a detected or inferred identity of one or more taxonomic classes of the colony (e.g. Gram status, genus, family, species, strain, etc.).

As illustrated below in FIGS. 11C and 11D, a selected size threshold (e.g. diameter threshold) may be employed to ensure that a sufficient number of microbial cells are harvested for a wide variety of cell classes (e.g. species). For example, as shown in FIGS. 11C and 11D, at least 10³ microbial cells can be obtained across a wide range of microbial cell species provided that a colony is harvested after reaching a diameter threshold of 70 microns, but prior to reaching a diameter of 100 microns (or alternatively, at least 65 microns but prior to reaching a diameter of 100 microns, at least 75 microns but prior to reaching a diameter of 100 microns, at least 80 microns but prior to reaching a diameter of 100 microns, at least 85 microns but prior to reaching a diameter of 100 microns, at least 90 microns but prior to reaching a diameter of 100 microns, or at least 100 microns but prior to reaching a diameter of 120 microns). Likewise, as shown in FIGS. 11C and 11D, at least 10⁵ microbial cells can be obtained across a wide range of microbial cell species provided that a colony is harvested after reaching a diameter threshold of 150 microns but prior to reaching a diameter of 200 microns (or alternatively, at least 165 microns but prior to reaching a diameter of 200 microns, at least 175 microns but prior to reaching a diameter of 200 microns, at least 180 microns but prior to reaching a diameter of 200 microns, at least 185 microns but prior to reaching a diameter of 200 microns, at least 190 microns but prior to reaching a diameter of 200 microns, or at least 200 microns but prior to reaching a diameter of 250 microns). Although the present example threshold embodiment refer to a diameter, it will be understood that other size measures, such as a radius or area, may be employed in the alternative.

It will be understood that optical imaging is but one example detection modality for monitoring the growth of microbial cells, and that other detection modalities, such as electrical impedance, the detection of volatile organic compounds associated with microbial cell growth, or calorimetry may be employed in the alternative.

As noted above, after having determined the presence of one or more colonies having a sufficient quantity of microbial cells, the microbial cells may be employed to perform one or more subsequent assays. The microbial cells may be harvested (removed) from the solid phase growth media prior to performing subsequent testing. For example, one or more of the detected colonies may be harvested (e.g. extracted using manual harvesting, automated harvesting, or a combination thereof) and subsequently processed such that they are provided in a form that is suitable for subsequent testing. For example, in some example implementations, the harvested microbial cells may be diluted or concentrated. In some example implementations, the harvested microbial cells may be combined with a liquid to form a suspension which may optionally be diluted or concentrated, and optionally aliquoted, prior to performing one or more assays.

In one example embodiment, having identified the location of the colonies, the colonies may be manually harvested by biopsy punch, inoculation loop or sterile cotton swabs. For some application, such as identification by MALDI the removed colony can placed on the identification slide. For some applications, such as antimicrobial susceptibility testing, the removed colony may be suspended in an appropriate medium such as saline solution.

In another example embodiment, having identified the location of the colonies, the colonies may be robotically harvested. For instance, using a circular instrument, similar to a biopsy punch, a small section of solid growth media may be removed with the microcolony. US Patent Publication No. 2018/0284146 has describes a device that is equipped with a platform for holding a culture plate and a movable robotic arm having a pick tool which can be lowered to pick colonies from the plate. In another part of the device, a sterile tube, containing a suspension media, is stored. The pick tool, after picking a part of the colony, moves and transfers the picked colony to the sterile tube. Optionally, the tool may be equipped with a sonicator (ultrasound transducer) or vortex mixer for more efficient release of the harvested microbial cells. The turbidity of the solution may then be measured and the cell concentration may be diluted to a predetermined value suitable for subsequent microbiology tests, such as antimicrobial susceptibility testing.

In some example embodiments, cells from one or more colonies or formed on the solid phase growth medium according to the preceding methods can be employed to perform AST. Example methods of performing AST include broth microdilution methods, disk diffusion, and agar diffusion methods such as the Kirby-Bauer method and the e-Test. Such methods may benefit from a presumptive (high-level, such as Gram status and fungal vs. bacterial determination) microbial identification for initial selection of antimicrobial panel.

FIG. 11A provides a flow chart describing an example method of performing AST based on microbial cells harvested from a colony or microcolony grown according to the methods described above. Cell isolation and seeding is first performed as per the example embodiments described above, in which microbial cells are separated from a whole blood sample, while maintaining their viability, and brought into contact with a solid phase growth medium enclosed inside a growth module. As described above, the process is advantageously performed in a closed manner for minimizing the possibility of contamination, especially for samples such as whole blood samples which are known to have very low microbial cell concentrations. The growth module is then housed within an incubation/imaging instrument (with the growth module optionally detached from a remainder of the integrated fluidic cartridge), where the growth module is incubated and monitored for detectable microcolonies, e.g. for determining positivity/negativity at time duration of <4 hours. Detected microcolonies may then be incubated to promote further growth in order to achieve a sufficiently high cell count for subsequent analytic steps (e.g. identification and/or antimicrobial susceptibility testing). For example, the microcolonies may be incubated until they are determined to have reached a cell content of >10⁴ and >10⁵ cells, which are, respectively, sufficient for running antimicrobial susceptibility testing (AST) and cell identification via MALDI. In one example embodiment, in which AST is performed according to the example methods described below, the minimum number of microbial cells in a harvested microcolony may be set to be in the range of 10³ to 10⁵. Accordingly, a given microcolony may be harvested after its inferred the cell content of has reached at least 10³. In one example implementation, this determination is performed by estimating the microcolony size via microscopy and estimating the lower bound of the microcolony cell content accordingly. In order to illustrate this example method of microcolony harvesting, a series of experiments were performed, as described below.

FIG. 11B plots the measured dependence of microcolony diameter no cell content for the example case of E. coli (obtained according to the method described in Example 7). The scatter graph has been fitted by a power law trend line, using which the average microcolony diameters with cell contents of 10³ and 10⁵ cells have been calculated to be respectively 60 μm and 170 μm. Following similar approach, the average diameters at 10³ and 10⁵ CFU cell content was calculated for 17 prevalent pathogenic gram positive and gram-negative bacteria and presented the results respectively in FIGS. 11C and 11D. According to this information, if a bacterial microcolony is harvested when its diameter reaches 65 μm, regardless of its identity, the number of microbial cells in the microcolony will be likely be within the range of 10³ to 10⁵ CFU.

As explained above, the cells of one or more detected microcolonies may be non-invasively interrogated, prior to harvesting, in order to determine a measure of a class of the microbial cells (e.g. a determination of Gram status, and a determination of bacterial vs. fungal cells, and/or preliminary species estimate).

A detected microcolony, having an associated class, may then be harvested and transferred from the growth chamber, re-suspended in a buffer to generate a microbial cell suspension, and employed for performing antimicrobial susceptibility testing. For example, as described in further detail below, aliquots (e.g. ~1 μL) of the microbial cell suspension may be dispensed onto a plurality of microwells containing solid phase growth media, the microwells containing a gel with a thickness ranging between 0.5 to 10 mm (or 0.5-3 mm) and gel volume ranging between from 20 to 150 μl (or 20 to 60 μl). The gel surfaces within the microwells may then be contacted with respective solid supports having varying concentrations of one or more antimicrobial agents disposed thereon (coated and/or impregnated). The antimicrobial agent rapidly diffuses into the microwell and the growth of microbial cells retained on the surfaces of the microwells may be monitored, for example, to determine a minimum inhibitory concentration (MIC). For example, due to the low volume and small spatial extent of the solid phase growth medium and the rapid diffusion of the antimicrobial agent (e.g. a prescribed concentration or concentration rage is achieved within 1-2 hours), a determination of which microwells support microbial cell growth and which microwells inhibit microbial cell growth enables a determination of a minimum inhibitory concentration (MIC) of each antimicrobial agent.

A second microcolony detected within the growth module may be further incubated, in parallel with performing the AST on the first harvested microcolony, and subsequently harvested after it has been determined to include a minimum number of cells for performing microbial cell identification (e.g. >10⁵ cells), for example, by employing a secondary (e.g. conventional) identification modality such as MALDI-TOF mass spectroscopy. The secondary identification modality may have a greater accuracy, greater confidence level, and/or a larger set of possible classes than the initial classification modality that was employed prior to the harvesting of microbial cells from the first microcolony. As shown in FIG. 11A, the harvesting of the second microcolony, and the secondary identification step, may be performed concurrently with the antimicrobial susceptibility testing that is performed on the first microcolony. In many cases, this approach will result in the identification results being made available prior to the AST results, such that the identification results can be reported along with the AST results for interpretation. For example, the identification results, known species-specific breakpoints, and other clinical factors may be employed when selecting an antibiotic and dose based on the AST results.

In some example embodiments, prior to harvesting microbial cells from the second microcolony, a phenotypic correspondence may be established between the first colony and the second colony. This phenotypic correspondence may be established, for example, by comparing classes associated with the two microcolonies, or, for example, comparing optical images or optical signals detected from the two microcolonies.

As noted above, in some example embodiments, AST may be performed on microbial cells harvested from one or more colonies, optionally after having performed at least a presumptive identification or classification of the colony microbial cell class (in some cases, presumptive identification may not be necessary, for example, if a colony is harvested after reaching a size known to have a minimum cell count across a wide variety of microbial cell classes, and if a broad AST panel is employed, for example, a panel that is sufficiently broad for to provide coverage for sets of both Gram positive and Gram negative bacteria). The harvested colonies (e.g. obtained using manual harvesting, automated harvesting, or a combination thereof) may then be suspended in a liquid to form a suspension (optionally diluted or concentrated), aliquoted and contacted with different concentrations of antibiotics.

The antibiotics (and optionally concentrations thereof) may be selected based on the identity of the microbial cells. For example, the aliquoted microbial cells may be contacted with three different concentration of the selected antibiotics and microbial cell growth may be subsequently monitored to determine a measure of susceptibility and/or resistance. In other example embodiments, additional concentrations of antibiotics may be employed.

Microbial Cell Separation and Subsequent Incubation in Liquid Growth Media While many of the preceding example embodiments pertain to methods that facilitate colony growth via contact of separated microbial cells with solid phase growth media, in another example implementation, after performing a separation process to obtain separated viable microbial cells, the separated viable microbial cells (optionally separated with one or more wash cycles) may be mixed with growth media in the liquid phase and incubated in an environment suitable for promoting microbial cell growth. For example, a suspension containing the separated microbial cells may be introduced into a blood culture bottle and incubated accordingly to a conventional blood culture incubation protocol (e.g. storage at 37° C. within an incubator). In the case of a sample associated with a patient who has been treated empirically with antibiotics prior to sampling, such an approach may facilitate the reduction of the concentration (and impact) of the antimicrobials with greater efficiency than that afforded merely by the inclusion of antimicrobial absorbing agents (e.g. charcoal or resins) in the blood culture bottle.

In one example implementation, liquid phase growth media may be combined with the separated cells within a closed cartridge and the closed cartridge may be subsequently incubated to promote the growth of microbial cells. Such an approach provides the benefit of avoiding contamination that could otherwise occur if the separated cells are transferred to an external cell culture vessel or device. If the closed cartridge includes a centrifugation chamber (as in the example embodiments described above), the cartridge could be periodically centrifuged (during incubation or in between incubation phases) and the distal region of the centrifugation chamber could be interrogated (e.g. optically via imaging or electrically via local impedance measurements based on internal electrodes (e.g. a circular array of electrodes) housed within the centrifugation chamber) to monitor the growth of the microbial cells. For example, the microbial cells collected in the distal region of the centrifuge tube may interrogated to determine whether or not a sufficient number of microbial cells are present to support subsequent antibiotic susceptibility testing. If an insufficient quantity of microbial cells is detected, the microbial cells may be resuspended and incubated for a given time duration prior to repeating the assessment.

In some example methods, viable microbial cells may be separated from liquid culture samples, such as a blood culture sample. In some example implementations, a blood culture sample (or another sample type that is cultured in a liquid phase) may be processed to obtain separated viable and/or intact microbial cells prior to a determination of positivity of the blood culture sample. The separated microbial cells may then be employed for one more subsequent assay, such as, but not limited to, MALDI identification, metabolic-assay-based identification, and/or phenotypic antimicrobial susceptibility testing (e.g. via broth microdilution or another phenotypic antimicrobial susceptibility testing method). In one example embodiment, a first portion of the separated microbial cells may be employed to perform microbial identification via MALDI and a second portion of the separated microbial cells may be employed to perform antimicrobial susceptibility testing (e.g. after having performed MALDI using the first portion of microbial cells).

The number of microbial cells that are required for subsequent assay processing will generally depend on the assay type. In general, it may be determined that a minimum quantity of microbial cells is required for a given assay. The quantity of microbial cells required for the given assay may also depend on the type of microbial cell (e.g. Gram status, genus, family, or species). In some example embodiments, a determination of whether or not a sufficient quantity of microbial cells has been obtained in the separated microbial cells may be made by performing a measurement on the separated microbial cells. For example, the separated microbial cells may be suspended and a turbidity measurement may be performed on the suspension. Alternatively, a measure of the quantity of separated cells may be determined, for example, using a modality selected from the following non-limiting example list: flow cytometry and electrical impedance measurements. The suspension of separated microbial cells may be concentrated in order to achieve a sufficient sensitivity of detection. For example, filtration and/or centrifugation, followed by resuspension in a sufficiently small volume of liquid, may be employed in order to achieve a suitable sensitivity of detection. The concentration that is required for a given optical detection modality may be determined by performing measurements on serially diluted aliquots from a concentrated stock of reference microbial cells. In one example embodiment, optical turbidity measurements may be made on a concentrated suspension of microbial cells, where the suspension is measured via laser scattering within a cuvette or other suitable vessel having side walls suitable for optical scattering measurements.

As described above in FIG. 9A-9F, the growth rate of microbial cells in growth media will generally depend on the microbial cell class at the species or genus level. Accordingly, an initial determination of microbial cell class at the genus or species level, using a rapid molecular identification assay, may be employed to estimate a suitable time to process a liquid culture sample in order to obtain a sufficiently high quantity of separated microbial cells for subsequent assay processing or for harvesting a growing microbial cell colony formed from separated cells. Non-limiting examples of suitable rapid identification assays include the example rapid rRNA-based identification assays described above and rapid gDNA assays such as the Septifast assay and the T2Bacteria assay. A blood culture sample may be obtained from a blood culture bottle (e.g. via obtaining a suitable aliquot from a blood culture bottle) at the suitable time corresponding to the presence of the sufficiently high quantity of microbial cells. In some example embodiments, the suitable time may be increased by an additional time duration such as a guardband time (e.g. 0.5 or 1 hours) and/or a prescribed multiple of a standard deviation of the time (e.g. 1 or 2 standard deviations).

In some example implementations, if the rapid identification assay is quantitative and provides a quantitative measure indicative of the concentration of the microbial cells in an initial sample (such as via the determination of a cycle threshold value during amplification), then the quantitative measure may be employed to refine the estimate of the suitable time for obtaining a liquid culture sample or for harvesting a growing microbial cell colony formed from separated cells, in order to obtain the desired quantity of microbial cells for the subsequent assay. For example, the quantitative measure may be combined with an initial identification result to obtain an estimate of the time that will elapse prior to a given event associated with microbial growth, such as, but not limited to, time to positivity and time to reach a pre-selected concentration.

### Examples of Microbial Cell Separation in an Integrated Fluidic Cartridge

An example automated system for performing microbial cell separation and concentration, based on the methods of International Patent Application No. PCT/CA2013/000992, is taught in International Patent Application No. PCT/CA2015/050449. FIG. 12 provides an illustration of the example integrated system 400 for performing automated centrifugal separation (and/or washing). The example system 400 includes a centrifuge 410, which receives one or more integrated fluidic processing cartridges 420 for centrifugal separation. The centrifuge 410 includes one or more receptacles 412 which are connected to a motorized rotor 414 and are configured to receive integrated fluidic processing cartridges 420. The cartridge receptacles 412 may be, for example, of the fixed angle type or the swinging bucket type which are common in laboratory centrifuges (e.g. each receptacle 412 may be pivotally connected to the motorized rotor 414).

The cartridge interface assembly (unit) 430 is configured to removably engage (or interface) with an integrated fluidic processing cartridge 420 when the motorized rotor 414 is at rest, for controlling the flow of fluids within integrated fluidic processing cartridge 420. The interfacing of the cartridge interfacing assembly 430 with the integrated fluidic cartridge may occur, for example, via a direct interface between the cartridge interfacing assembly and the integrated fluidic cartridge 420, or, for example, via an interface (e.g. an actuation interface) on the centrifuge 410 (e.g. on the motorized rotor 414 or cartridge receptacle 412).

The centrifuge 410 and the cartridge interfacing assembly 430 are controlled via control and processing unit 440. The control and processing unit 440 may include one or more processors 445 (for example, a CPU/microprocessor), bus 442, memory 455, which may include random access memory (RAM) and/or read only memory (ROM), one or more internal storage devices 450 (e.g. a hard disk drive, compact disk drive or internal flash memory), a power supply 480, one more communications interfaces 460, external storage 165, a display 470 and various input/output devices and/or interfaces 475 (e.g., a receiver, a transmitter, a speaker, a display, an output port, a user input device, such as a keyboard, a keypad, a mouse, a position tracked stylus, a position tracked probe, a foot switch, and/or a microphone for capturing speech commands).

According to the teachings of International Patent Application No. PCT/CA2015/050449, and with reference to the example schematic representation in FIGS. 13A to 13E, an example integrated fluidic processing cartridge 500 is illustrated which incorporates elements suitable for automated separation and washing of microbial cells from whole blood to obtain a concentrated suspension. The example integrated fluidic processing cartridge includes a sample transfer receptacle 501, a macrofluidic centrifugation chamber 502, a diluent chamber 504 and a supernatant chamber 506. Diluent chamber 504 is prefilled with a wash buffer fluid 505, is fluidically connected to macrofluidic centrifugation chamber 502 via conduit 510 equipped with shutoff valve 512, contains a vent to atmosphere 515 and is otherwise closed. The supernatant chamber 506 is fluidically connected to macrofluidic centrifugation chamber 502 via a conduit 511 equipped with shutoff valve 513, and contains a vent to atmosphere 516, where the supernatant chamber 506 is otherwise closed. The macrofluidic centrifugation chamber 502 has a conical or round bottom shape and a smooth inner surface which minimizes adsorption or trapping of microbial cells during centrifugation and is closed with the exception of the openings 522, 523, 524, 525, 526 to respective conduits. In the present example embodiment, the macrofluidic centrifugation chamber is employed for the processing of blood-containing samples (e.g. whole blood, blood culture samples, or other blood-containing samples), and contains a blood lysis reagent 503 and a cushioning fluid 529 to aid in microbial cell recovery and to minimize compaction injury of the cells which may compromise the integrity and recovery of the target nucleic acids.

The sample transfer receptacle is equipped with a needle 507 which is mounted at the bottom of the receptacle. The needle is connected to a fluid path 508 equipped with a shut-off valve 509 which leads to macrofluidic centrifugation chamber 502. A sample tube or container 520 with a pierceable cap 521, such as, for example a Vacutainer® blood collection tube or a blood culture tube containing a blood sample and growth media, may be inserted into the sample transfer receptacle such that the needle 507 pierces the cap 521 thus allowing transfer of a sample fluid to the cartridge via the needle and fluidic path 508. Optionally, the needle 507 is covered with a pierceable hood 508 which protects the needle from contamination.

The example integrated fluidic processing cartridge 500 taught by International Patent Application No. PCT/CA2015/050449 is a closed cartridge (apart from the vents described below) which, following the insertion of the sample, performs all the functions required for separation and washing of a concentrated suspension within the chambers and conduits of the cartridge, has all reagents and solutions stored in chambers on the cartridge, and retains all excess liquids including waste supernatant in chambers on the cartridge. One or more of the vents and ports may be protected by air permeable membranes with a pore size sufficiently small to prevent the ingress of microbial pathogens in the target range of the device.

According to the present example embodiment, all excess and waste liquids are stored on the cartridge and are not exposed to the user. Thus, the closed cartridge provides a device which protect the user from direct contact with the sample and for which the sample is not susceptible to contamination by external factors during the separation and washing process.

As taught by International Patent Application No. PCT/CA2015/050449, an automated separation and washing process is generally described in FIG. 14, with reference to the example integrated fluidic processing cartridge 500 shown in FIG. 13A. A cartridge interfacing assembly, described in detail in International Patent Application No. PCT/CA2015/050449, is equipped with all the components required to perform the necessary actions including actuation of the cartridge valves 509, 512, 513, and 517 and an air displacement device capable of application of both positive and negative gauge pressure to the cartridge centrifuge chamber via cartridge port 518.

The sample tube 520 containing a sample is inserted into the sample transfer receptacle 501 of cartridge 500 thus piercing the tube cap 521 to perform the sample transfer to the macrofluidic centrifugation chamber as shown at 502 of FIG. 13A. The cartridge interface assembly engages with the cartridge via a cartridge receptacle, described in detail below, and is actuated such that valve 509 is open and valves 512, 513 and 517 are closed, thus sealing all fluid paths emanating from macrofluidic centrifugation chamber except the path 508 from the sample tube.

An air displacement device is engaged with the port 518 by way of a connector which provides a sealed connection with the port. Optionally, a rigid or flexible tube connects the air displacement device to the connector. Sample transfer to macrofluidic centrifugation chamber 502 is performed by operating the air displacement device to extract air from macrofluidic centrifugation chamber to cause sample flow from the sample tube 520 into macrofluidic centrifugation chamber 502 via fluid path 508. The entry 523 of the port 518 must be positioned above the fluid level and with a sufficient air gap between the fluid level and the entry 523 such that no fluid flows into entry 523 to the port 518. The air displacement activated flow is done in a controlled manner such that a predetermined volume of sample is transferred into macrofluidic centrifugation chamber.

According to one embodiment of the teachings of International Patent Application No. PCT/CA2015/050449, the entry 522 to flow path 508 is also in the air gap above the fluid level such that, following transfer of the desired volume of sample, the air displacement via port 518 can be reversed to provide a small amount of air displacement into macrofluidic centrifugation chamber to clear the flow path 508 of sample fluid and move this residual sample back into the sample tube 520. Then the valve 509 is closed and the sample tube 520 is optionally removed from the receptacle 501.

The blood lysis reagent 503 may be present in the centrifugation chamber 502 prior to the sample transfer process or alternatively it may be transferred from a blood lysis reagent tube in a similar manner as the sample. Alternatively, a blood lysis reagent storage chamber may be provided on the cartridge and a fluidic path with valve and an air vent may be provided to allow the blood lysis reagent 503 to be moved to macrofluidic centrifugation chamber in a similar manner to the movement of wash buffer to macrofluidic centrifugation chamber as described below.

As taught in International Patent Application No. PCT/CA2015/050449, after addition of the sample to macrofluidic centrifugation chamber 502, the sample and the blood lysis reagent 503 may optionally be mixed as shown at 905 in FIG. 14. A mixing mechanism may be provided whereby the instrument performs vortexing, shaking, or cyclic inversion of the cartridge. This operation is performed with valves closed on all fluid paths emanating from macrofluidic centrifugation chamber 502. A valve may be provided on the fluid path to the port 518 to prevent fluid from entering the air path during mixing. In addition, or alternatively, an air permeable membrane which prevents the passage of fluid may be placed in the air path between macrofluidic centrifugation chamber and the port 518 to prevent fluid from reaching the port 518. This membrane may also be configured to serve as an air filter to prevent the ingress of microbes from the environment or from the air displacement device. Alternatively, the path between the port 518 and the entry opening 523 to the macrofluidic centrifugation chamber can be designed to possess high fluidic resistance such that under the prevailing conditions fluid will be prevented from entering the opening 523 or will be prevented from proceeding all the way to the port 518. Likewise vents 515 and 516 in diluent chamber 505 and supernatant chamber 506 respectively may be equipped with an air permeable membrane and/or a path with high fluidic resistance to serve a similar purpose.

Following the mixing step 905, a centrifugal sedimentation step 910 is performed whereby the cartridge interfacing assembly is disengaged from the motorized rotor 414 and the cartridge 420 is centrifuged such that the microbial cells in the macrofluidic centrifugation chamber sediment on the cushioning liquid, for example, as per the methods of PCT Patent Application No. PCT/CA2013/000992, as described above. The centrifuge may be, for example, an angle centrifuge or a hanging bucket centrifuge and the centrifugal parameters may be selected, for example, according to the conditions provided in PCT Patent Application No. PCT/CA2013/000992.

The relative centrifugal force applied to the fluids within the macrofluidic centrifugation vessel may be, for example, within the range of 1000-15,000 g, or for example, 2,000 -12,000 g, or, for example, 3000-10,000 g, or, for example, 3000-7,000 g, or, for example, 5000-10,000 g, or, for example, 4000-8,000 g. In applications involving separation of bacterial and fungal cells from biological samples, it has been found that a suitable relative centrifugal force (RCF) is within the range of 1000 g-15000 g range, and more specifically, within the range of 3000 g-7000 g.

Following the centrifugal sedimentation step 910 of FIG. 14, the centrifuge rotor is stopped and the cartridge interfacing assembly is re-engaged with the motorized rotor as shown at 915 and extraction of the supernatant 527 from macrofluidic centrifugation chamber 502 to the supernatant chamber 506 is performed as shown at 920, whereby the residual 528 (containing the microbial cells) is retained at the bottom of macrofluidic centrifugation chamber 502. This action is performed by opening valve 513 while valves 509, 512 and 517 remain closed and engaging the air displacement device connector with port 518 and controllably displacing air into macrofluidic centrifugation chamber. Thus, air displacement induced flow of the supernatant occurs through fluid path 511, the entry 524 of which is placed below the lowest extent of the supernatant. Optionally the entry 524 is placed at the lowest extent of the supernatant which is to be expressed from macrofluidic centrifugation chamber, thus preventing residual 528 from being extracted from macrofluidic centrifugation chamber.

Following the supernatant extraction step 920, the wash buffer dispensing steps 925 and 930 are performed whereby wash buffer is dispensed into macrofluidic centrifugation chamber 502. This action is performed by opening valve 512 while holding valves 509, 513 and 517 closed and engaging the air displacement device connector with port 518 and controllably evacuating air from macrofluidic centrifugation chamber 502. Thus, air displacement induced flow of the wash buffer occurs through fluid path 510. The entry 525 of wash buffer path 510 is preferably placed above the highest extent of the fluid level in macrofluidic centrifugation chamber.

Following the wash buffer dispensing step 544, the mixing step 932 is performed to thoroughly mix the wash buffer and the residual fluid in macrofluidic centrifugation chamber. This may be performed by vortexing, shaking, or cyclic inversion of the cartridge as described previously. Following the mixing step 932, the centrifugal sedimentation step 910 is performed to re-sediment the collected microbial cells and the supernatant is removed from the centrifugal chamber as in step 920. The sequence of steps 925-935 and 910-920 collectively form a wash cycle, whereby the cell suspension is diluted in wash buffer, the microbial cells are re-sedimented, and the supernatant is extracted. The wash cycle may be repeated multiple times to effect multiple additional wash cycles as required to obtain a final microbial cell suspension that is sufficiently dilute of contaminants and interferants. Following the final supernatant extraction step 920, the mixing step 942 is performed to resuspend the sedimented microbial cells in the final residual fluid 528 to produce the final suspension. Following the resuspension step 942, the final suspension is extracted by air displacement through fluid path 510. The volume of the final suspension depends on the nature of the application. For instance, when the intended application is the detection of microbial cells in whole or cultured blood, the volume of the final cell suspension may be selected to be in 10 μL-500 μL range, while a more preferred range is 20 μL-120 μL, or 50-100 μL. During the extraction of the final cell suspension valve 517 is open and valves 509, 512 and 513 are closed and air is displaced through port 518 into macrofluidic centrifugation chamber to displace the fluid out of opening 526 via fluid path 516 to port 519. The opening 526 is so positioned at the top surface of the cushioning fluid 529 that the final suspension in its entirety, or substantially all of the suspension, is expressed from macrofluidic centrifugation chamber without expressing any of the cushioning fluid 529. Alternatively, the opening 526 is so positioned that the final suspension and a portion of, or all of, the cushioning fluid may be expressed from the macrofluidic centrifugation chamber through fluid path 516. The fluid path 516 is fluidically connected to the cell colony growth module inlet path 101 and 161 as described in FIG. 2.

FIGS. 13B and 13C illustrate an example integrated cartridge for performing automated sample preparation including separation of microbial cells from a sample and seeding them onto a solid phase growth media in a closed cartridge configuration. The example integrated cartridge 700 (FIG. 13B) is shown having three components. The first component 698 includes the sample transfer receptacle 501, macrofluidic centrifugation chamber 502, the diluent chamber 504 and supernatant chamber 506 (referred to FIG. 13A). The first component 698 may be a single plastic molded part fabricated from materials which are compatible with the form and function of the device. Alternatively, the first component 698 may be an assembly of subcomponents which are plastic parts, molded or formed by a means consistent with the material, form and function of the device. In this respect, the material should be selected to be of sufficiently high strength to withstand the high centrifugal forces that the cartridge will be subjected to, and the materials should be compatible with the fluids used and, in the case of molecular applications, should not introduce contaminants into the pretreated cell suspension which will interfere with downstream process. Non-limiting examples of materials from which first component 698 can be fabricated are polypropylene, polycarbonate, polyethylene, PET, polystyrene, Cyclic Olefin Copolymer or some variant of these materials.

The second component 699 is a microfluidic device mounted on the lateral face of component 698. The second component 699 comprises fluidic paths and valves connecting the chambers in component 698 and the cell colony growth module 720. The fluidic paths and components are for flowing the cell suspension from the cell suspension path 516 and 519 (referred to FIG. 13A) to the cell colony growth chamber and additional components for seeding the growth media as previously described herein. The component 699 is a laminate composed of a number of layers in which are formed holes, channels and chambers. The layers may be machined, punched, embossed or molded to form the necessary features. Each layer may be comprised of either a single or multiple sublayer each of either different materials or the same materials listed previously based on the function of the sublayer laminated by either adhesives bonding, thermal bonding, ultrasonic bonding, or other methods known to those skilled in the art.

In one embodiment, as illustrated in FIG. 13B, the cell colony growth module 720 may be incorporated with the laminate 699 as a removable module so that the growth module may be separated from the remainder of the cartridge for subsequent processing. The removable colony growth module may include a number of features which facilitate removal of the module from the cartridge such as finger tabs and snap features which secure the module but are easily broken for ease of removal, a breakable or otherwise detachable fluidic connection to the remainder of the laminate 699. In addition, the colony growth module include a transparent backing material having a set of grids engraved therein or marked thereon, as schematically illustrated in FIG. 13E. This feature will help for locating microcolony to be harvested for other applications without requiring the visualization of microcolony.

In one embodiment, as illustrated in FIG. 13D, the cell colony growth module 720 may be incorporated into the second component 699 and the first component 698 such that it resides perpendicular to the centrifugal field for the purpose of spreading the sample on the gel. One example embodiment that facilitates fluidic connection of the second component (laminate) 699 to the cell colony growth module 720 is via a breakable laminate tab 725, which for instance can be locally bonded via laser welding or pressure sensitive adhesives to the 699 and 720 at the location of the fluidic connections 721 and 722 in such a way that it is strong enough to withstand the operational loads, however a user can peel part 725 off the laminate 699 thus freeing 720 from the cartridge. Optionally the part of 725 which is now free can subsequently be re-bonded to 720, via a pressure sensitive adhesive which is exposed once 720 is removed from the cartridge in order to maintain the sealed environment within 720.

The openings 710 (shown in FIG. 13B) of the chambers of the cartridge may be sealed with a membrane seal, a foil seal or a cap 697 (shown in FIG. 13C) following dispensing of the wash buffer and pretreatment fluid into the diluent chamber and macrofluidic centrifugation chamber respectively. FIG. 13C shows the outer surfaces 703, 704 and 705 of the centrifugation chamber, the diluent chamber, and the waste chamber, respectively. The seals or caps may be bonded using methods and materials compatible with heat sealing, adhesive bonding, ultrasonic bonding. Alternatively, the chambers may be sealed prior to dispensing of these liquids and alternate ports may be provided for the purpose of dispensing these liquids and these ports may be sealed following the dispense operation. The cap 697 may be molded, embossed, machined or rapid prototyped, and may be constructed from polycarbonate, polystyrene, PET, polyester or other material appropriate to its form and function.

### Example of a System for Microcolony Detection and Performing Presumptive Identification

An example microbial incubation and monitoring system for incubating and detecting microcolonies and optionally performing presumptive identification is schematically presented in FIG. 15A. The system includes an open or closed incubation chamber 81, which may be closed by a removeable or sliding lid 82, and which houses one or more growth modules 720. The lid 82 may be transparent and sufficiently flat to avoid image distortion. The lid 82 may be heated to prevent condensation. The lid may include an opening for an ‘immersion’nosepiece. Additionally or alternatively, objectives (e.g. one or more long working distance objective) may be used. A heater, temperature sensor and associated control circuitry may be employed to maintain temperature within an acceptable range relative a set temperature, (e.g. 37° C.). The gas composition and ambient humidity may also be regulated by connecting gas inlet and outlet ports 83 to one or more suitable external modules (e.g. gas mixture to control CO₂/O₂ to provide appropriate aerobic or anaerobic atmosphere; a reservoir for water to control humidity). The complete system may be enclosed while the temperature, gas composition and humidity are controlled via recirculation. The chamber may include one or more retention devices (e.g. clips or clamps) for firmly holding the growth modules 720.

The example system is equipped with at least two imaging modules. A first imaging module 84 is provided having a first field of view and associated magnification and a second imaging module 85 is provided having a second field of view and associated magnification, where the second imaging module has a smaller field of view and a higher magnification than the first imaging module. The imaging system may be provided with rapid autofocus capabilities, e.g. via a linear motor that is driven according to contrast-based feedback associated with one or more images. The imaging modules may include an objective heater for ‘immersion’ optics. After placing a growth module 720 inside the chamber, the first imaging module 84 is controlled by drive actuators (e.g. motors) and the control and processing circuitry 86 (such as the example control and processing circuitry 440 shown in FIG. 12) such that at least a portion of the surface of the solid phase growth medium is within the field of view.

In cases in which the field of view is smaller than the full surface of the solid phase growth medium, the imaging module may be mechanically scanned during imaging and the images may be combined using control and processing circuitry.

This task may be accomplished by processing overlapping image tiles from multiple fields of view (FOVs), stitching the overlapping image tiles together, thereby enabling studies of a large region (e.g. the entire region) of the growth module via large 2D time-lapse mosaics. Due to potential inaccuracy in the system, a misaligned individual FOV may create a misalignment in final mosaic image, which can lead to errors and loss of information. In some example implementations, the control and processing circuitry 86 could compensate for the mechanical imprecision (linear motor backlash, and stage repeatability) and this avoid or minimize stitching errors by optimizing the translations within a specified area via pairwise registration with a specified transformation constrains (e.g. translation only or translation+rotation). For example, an intensity-based or feature-based algorithm may be employed generate a transformation among adjacent images, such that adjacent images are spatially registered. In this context, the stage trajectory function may provide an initial mapping between adjacent image tile, which is refined via image registration.

The second imaging module 85, which exhibits a higher magnification than the first imaging module 84, may be optionally equipped with epi-illumination for supplementary dark-field imaging. Once the colonies are detected and located via images obtained from the first imaging module 84, the control and processing circuitry 86 may control the second imaging module such that the detected microcolony is imaged by the second imaging system. After focus adjustment, higher resolution images (i.e. with a higher resolution than images obtained using the first imaging module) may be acquired, for example, to collect images for performing presumptive identification based on or more properties of the acquired images (e.g. one or more spatial, morphological, and/or diffractive parameters of the imaged colony, optionally based further on time-dependent changes in such parameters, or via an imaging modularity such as Raman microscopy or Fourier transform infrared microscopy that employs the second imaging module, as previously described). The present example system, or variations thereof, may be employed for imaging microorganisms tagged with fluorescent labels and/or unlabeled microorganisms.

One example implementation of a microcolony incubation and detection system is presented in FIG. 15B. The incubation chamber, which is supported on a translation stage is heated from the base and is closed by a sliding lid 82. The translation stage, in addition to moving in x and y directions, can also move in z direction for enabling autofocusing on the gel surface, for example by attempting to sharpen the images of the blood debris transferred from the sample. The objective 84 is passed through the opening 821 provided in the lid 82. The condensation on the objective is avoided by heating it to an elevated temperature (compared to the temperature inside the chamber) via a collar heater 841. The humidity inside the chamber is kept elevated by placing a partially water-filled open vessel 725 within the chamber.

In order to demonstrate the suitability of the system for detecting microcolonies, 4 mL of whole blood, spiked with about 20 CFU of E. coli cells, was processed according to the method of Example 5, to obtain a separated cell suspension. An agar plate, which had been prepared following the method of Example 6, was centrifuged for 8 minutes and the cell suspension was dispensed on it and allowed to be absorbed on the surface. The agar plate was placed in the incubator of FIG. 15B and was imaged every hour by taking 448 images across the gel surface. The 5× objective was moved in z direction for autofocusing at each fifth imaging event to reduce the scanning time. The collected images were aligned, registered, and stitched, and the result was presented in the right side of FIG. 15C. The plate was then incubated overnight and its image was taken by a conventional camera and was presented at the left side of FIG. 15C. The microcolonies, despite being undetectable with the unaided eye, were detectable in the images collected using the 5× objective at t=4 after sample seeding. One exception was colony #19, which was not detected because of shadowing by the plate's wall.

In FIG. 15D, detailed microscopic images of the 18 locations on the plate where microcolonies were detected are shown. At the bottom side of the image, the background-subtracted images of the colony containing regions at incubation times t=3 and t=4 hours are shown. As can be seen, by t=2 h one microcolony is detected (microcolony 7), while by t=3 h a total of 15 microcolonies out of the 18 microcolonies have been detected. The 3 h mark is still above the time to positivity (TTP) of 2.1 h that was estimated and reported in FIG. 9C. This reduced performance is attributed to the fact that the gel surface undergoes micro-scale morphology changes soon after being placed inside the incubator of the imaging system. Therefore, the image at t=0 h may not be the most suitable reference image for distinguishing the target microcolonies from the background debris. In order to circumvent this issue, the first (reference) image may be taken 10-30 minutes after incubation. Alternatively, the performance in terms of TTP may be improved by performing scans and collecting images at shorter time intervals (e.g. every 0.5 hour versus every 1 hour).

It is noted that although the system of FIG. 15B does not provide an actively managed/controlled CO₂ environment, the growth of bacterial species such as Streptococcus pneumoniae has not been found to be significantly impacted. Indeed, experiments involving 1 ATCC strain and 4 different clinical isolates of Streptococcus pneumoniae, it was found that the growth rate of Streptococcus pneumoniae in air ambient was approximately 2 cycles/hour for all strains. As it can be observed from FIGS. 9B and 9C, this growth rate is just at the lower end of the growth rate for typical pathogenic gram-positive bacteria. However, the fact that all bacterial and fungal cells can be detected in a similar atmosphere, without requiring the addition of CO₂, may provide a significant advantage, and may be attributed to the lower packing of the cells, consequently less cell-cell interactions, at smaller colony sizes.

In order to illustrate some evidence for this assertion, in the top portion of FIG. 15E images of colonies Streptococcus pneumoniae are shown, where the colonies were incubated overnight in the presence of (left) and the absence of (right) a CO₂ pack. As it is noticed, the colony incubated in the presence of the CO₂ pack appears healthier, as there is no visible region of cell depletion across the colony, in contrast to the colony incubated in the absence of the CO₂ pack. The bottom portion of the figure shows images of microcolonies (left) and a specific microcolony (right) incubated using the system shown in FIG. 15E, in the absence of a CO₂ pack. As can be seen in the image of the microcolony, the morphology of the microcolony is similar to the case of colony that was incubated overnight in the presence of the CO₂ pack.

### Methods of Performing Rapid Phenotypic Antimicrobial Susceptibility Testing

The forthcoming section of the present disclosure addresses shortcomings of conventional antimicrobial susceptibility testing (AST) methods such as microdilution assays and disk diffusion assays, and presents example embodiments for rapidly assessing the effect of a chemical agent, such as an antimicrobial agent, on a microbial cell. As explained below, the present example systems and methods may permit a determination of antimicrobial sensitivity (including permitting a determination of minimum inhibitory concentration) within a time duration of 4 hours for many microbial cell species, even for microbial cell counts as low as 10⁴ CFU, or as low as 10³ CFU.

The current gold standard for testing the sensitivity or resistance of bacteria to antimicrobial drugs for bacteria is a semi-quantitative in vitro susceptibility testing by the agar diffusion test procedure according to the standardized Kirby-Bauer method. The disk diffusion AST (DD-AST) test methodology (based on Kirby-Bauer method/Stokes method) involves seeding microbial cells on an agar plate and placing conventional 6-mm paper disks impregnated with specific concentrations of antimicrobial agents. The rate of diffusion and extraction of the antimicrobial drug out of the disk is not rapid. Therefore, concentration gradient is present with the highest concentration closest to the disk and logarithmic reduction with the distance from the disk. During the colony growth of microbial cells at given location with respect to the center of the disk, the local concentration of the antimicrobial agent evolves over time.

Typically, the inhibitory effect of the antimicrobial is manifested as the visible absence of a microbial lane up to a distance from the center, known as zone of inhibition, as illustrated with the notation 2r₁ in FIG. 16 for the case of Staphylococcus aureus in the presence of three antimicrobial agents, Oxacillin 1 μg/mL, Tetracycline 30 μg/mL, and Norfloxacin 10 μg/mL. Beyond this distance, sparse lane is observed up to a distance r₂, beyond which the lane is full. The test is time consuming, taking 18-30 hours, and requires a high bacteria concentration (turbidity that exceeds or is equivalent to the 0.5 McFarland). Moreover, the results are evaluated subjectively via a visual inspection determining a zone of a complete inhibition (2r₁) and recording the diameter of the zone in mm. Inaccuracy in MIC determination can occur when a zone of a complete inhibition is be not circular, as in the case of Tetracycline shown in FIG. 16. Accordingly, r₁ is specified within a range of values. As a consequence of these shortcomings, the disk diffusion AST method is a semi-quantitative and slow test. Nonetheless, the test is phenotypic as the scattering of light from the microcolonies enables visual inspection, and consequently the inference of the inhibition of microbial cell growth is unambiguous. In addition, storage of antimicrobial agent in dry form and its effective release upon the contact of the disk with the gel surface renders the test easy to perform.

When performing microdilution AST, aliquots of microbial cell suspensions are incubated in the presence of multiple concentrations of the antimicrobial agent, typically differing by factors of 2, and the growth rate is assessed with respect to the growth rate in the absence of the antimicrobial agent, either by monitoring the microbial concentration over the course of the test or at the end point. The monitoring approach at early stages of the test requires addition of signal generating agents, such as enzymes, which monitor the cell metabolic activity, and thereby is not direct indication of the cell proliferation. The end point assay (performed by measuring light scattering) is only sensitive for high microbial loads and can give satisfactory results after long incubation times, typically 10 hours or longer. In addition, the starting cell concentration in the sample should not fall below a required concentration, as has been illustrated in published art [Smith, Kenneth P., and James E. Kirby. “The Inoculum Effect in the Era of Multidrug Resistance: Minor Differences in Inoculum Have Dramatic Effect on Minimal Inhibitory Concentration Determination.” Antimicrobial agents and chemotherapy (2018): AAC-00433]. Thus, the sample is often obtained from a concentrated sample, typically with turbidity that exceeds or is equivalent to the 0.5 McFarland. Despite the mentioned issues, microdilution provides MIC values for quantitatively assessing antimicrobial susceptibility.

The present disclosure provides improved systems, devices and methods for assessing the effect of a chemical agent on microbial cells, utilizing the advantageous properties of the both disk diffusion AST and microbroth dilution AST, while avoiding many of their respective shortcomings. In several example embodiments disclosed herein, AST is performed by contacting a solid phase growth medium with a solid support having an antimicrobial agent dried thereon or impregnated therein, in a configuration such that the antimicrobial agent laterally diffuses inwardly from the solid support to a subregion of a solid phase growth medium that is at least partially surrounded by the solid support, to rapidly establish a local concentration of the antimicrobial agent, in contrast to the outward diffusion modality employed in to the disk diffusion AST method in which the antimicrobial agent laterally diffuses radially outwardly from the disk. By rapidly establishing a concentration of the antimicrobial agent within a subregion of the solid phase growth medium via local lateral diffusion of the antimicrobial agent, microbial cells dispensed onto surface of the subregion can be rapid exposed to the antimicrobial agent for the rapid assessment of the impact of the antimicrobial agent on the growth of microbial cells (e.g. via overhead optical imaging), thereby facilitating rapid phenotypic AST. This rapid phenotypic modality, which is facilitated by the local lateral diffusion of antimicrobial agent, in contrast to the conventional disk diffusion methods that rely on the global outward lateral diffusion of the antimicrobial agent, is henceforth referred to as “local diffusion” AST, or LD-AST.

The differentiation of the LD-AST assay relative to the conventional disk diffusion assay may be understood by referring to FIGS. 17A and 17B, which provide overhead views of the respective disks that are employed to facilitate AST. In the case of the disk diffusion AST (FIG. 17A), the antimicrobial is impregnated on a paper or plastic disk, 210, and the interaction and the susceptibility is evaluated by interrogating the growth of microbial cells which are exposed to the drug released from the disk that diffuses laterally outward to the regions on the agar plate beyond the disk. The region of interest for performing AST, 211, is an annular area having an internal area with a radius of the disk, rdisk, and outer unconfined radius of approximately three times the typical 3 mm radius of the disk.

In contrast, in the case of an example embodiment of LD-AST that is shown in FIG. 17B, the antimicrobial agent is provided on and/or impregnated within a surface of an annular disk 220 (a solid support, such as paper or plastic), the annular disk 220 having a central aperture (central open region) provided therein and the disk is contacted with a solid phase growth medium such that the antimicrobial agent is released from the annular disk and diffuses, at least in part, laterally (i.e. as opposed to mere vertical transport in a direction perpendicular to the planar surface of the solid phase growth medium) within the inner subregion that is surrounded by the annular disk. The surface 240 on which the antimicrobial agent is provided on and/or impregnated therein is referred to as the “contact surface”, as this surface is contacted with the solid phase growth medium to diffusively transfer the antimicrobial agent laterally within the solid phase growth medium. In the example embodiment shown in FIG. 17B, the contact surface is the bottom surface of the annular disk 220 that contacts the surface of the solid phase growth medium.

A droplet of microbial cell suspension is dispensed onto the subregion of the growth medium and microbial cells within the droplet are retained on the surface of the subregion (e.g. via evaporation and/or absorption of the droplet). The retained cells are thus exposed to the local concentration of the antimicrobial agent that is established via the local inward lateral diffusion of the antimicrobial agent from the contact surface of the annular disk, and the effect of the local antimicrobial agent concentration on the microbial cells is determined by incubating the structure and monitoring the cells for growth (e.g. via imaging of the subregion, from above, through the aperture). Accordingly, in the present example embodiment, the region of interest for assessing AST 221 is the surface of the subregion of the solid phase that is surrounded by the annular disk. By employing an annular disk having a small inner diameter, for example, less than 2 mm, or less than 1.5 mm, or less than 1 mm, the local concentration of the antimicrobial agent below the surface of the subregion is rapidly established (e.g. within 2 hours, within 1.5 hours, 1 hour, or 0.5 hours), thereby permitting the rapid assessment of microbial cell growth in the presence of the antimicrobial agent via optical microscopy.

When comparing the disk diffusion method of FIG. 17A to the local diffusion method of FIG. 17B, it is readily apparent that a significant difference between these two implementations is the significant reduction in the lateral distance over which the antimicrobial diffusion occurs in the case of the local diffusion AST method. In the case of DD-AST, the antimicrobial agent is continuously diffusing laterally outwardly, such that the antimicrobial agent diffuses unidirectionally (radially outwardly) within any given location within the solid phase growth medium that resides beyond the outer diameter of the disk. In contrast, in the case of LD-AST, while a portion of the antimicrobial agent diffuses laterally outward, another portion of the antimicrobial agent diffuses laterally inwardly into the subregion of the solid phase growth medium that is surrounded by the annular disk, with the consequence that the antimicrobial agent is delivered to the subregion from multiple, inward, radial directions, thereby facilitating the rapid and controlled generation of a near-spatially-uniform concentration of antimicrobial agent within the subregion.

The advantageous aspects of the present example embodiments can be further understood by considering the relevant timescales for diffusing of antimicrobial agents in solid phase growth media such as an agar-based gel growth medium. The characteristic length scale for concentration homogenization across one dimension is given by λ=sqrt(2*D*T_{D}), where sqrt stands for the squared root, T_{D} is characteristic diffusion time, and D is diffusion coefficient. The relevant diffusion time is over 1 hour, as most of pathogenic bacterial cells have a lag phase of about 1 hour before starting logarithmic phase growth. Values of D tabulated by Stewart (Stewart, Philip S. “Theoretical aspects of antibiotic diffusion into microbial biofilms.” Antimicrobial agents and chemotherapy 40.11(1996 ): 2517-2522.) were employed to estimate diffusion times for DD-AST and LD-AST. The typical value of D for an antimicrobial agent is approximately 5×10⁻⁴ mm²/s, with the result that a drug molecule is typically displaced by approximately sqrt(2×5×10⁻⁴×3600)~2 mm in one hour.

This result implies that in the case of LD-AST, the concentration of the antimicrobial agent that is established below the surface of the subregion that is surrounded by the annular disc is expected to become approximately spatially uniform in less than 1 hour. On the other hand, it will take approximately 10 hours for the antimicrobial agent to diffuse to the area of interest in the case of disk diffusion AST. Thus, employing inward lateral diffusion as a means of antimicrobial agent exposure within the central region of an annular disk, while allowing easy storage of the drug on the disk, also allows substantially reducing the time for establishing a uniform antimicrobial agent concentration over the region of interest. In addition, as described in further detail below, by controlling and configuring other aspects of the LD-AST platform, the time-dependent evolution of the antimicrobial agent concentration within the subregion may be tailored such that variations in concentration, both spatially and temporally, are less than 10% over timescales greater than 1 hour or even two hours.

This ability to reduce spatial and temporal variations in the concentration of the antimicrobial agent renders the present LD-AST platform to facilitate quantitative AST measurements. As described in further detail below, when multiple LD-AST devices are employed to generate different local antimicrobial concentrations, the effect of the antimicrobial agent on the growth of microbial cells may be discretely evaluated, in a manner similar to that of microbroth dilution AST, thereby facilitating the quantitative determination of a minimum inhibitory concentration from the set of discrete measurements.

The difference in the size of the region of interest employed in conventional disk diffusion AST (the large region surrounding the outer diameter of the disk) and LD-AST (the comparatively small subregion surrounded by the annular disk) also has a clear advantage in terms of reducing the required concentration for the sample.

The requirement on keeping bacterial cell concentration in an appropriate range has been illustrated in published art [Smith, Kenneth P., and James E. Kirby. “The Inoculum Effect in the Era of Multidrug Resistance: Minor Differences in Inoculum Have Dramatic Effect on Minimal Inhibitory Concentration Determination.” Antimicrobial agents and chemotherapy (2018): AAC-00433]. However, this requirement may be more relaxed in the case of performing the antimicrobial susceptibility testing on the solid phase.

The region of the solid phase growth medium into which the antimicrobial agent inwardly and laterally diffuses, and which is surrounded by the annular disk (or more generally, at least partially surrounded by a solid support, as described further below) which can have an associated surface area in the range of 0.5 mm² to 2 mm², is known in the context of the present disclosure as the “exposure region”, the “region of interest”, or “the subregion” of the solid phase growth medium. The present inventors have found that in some cases, an approximately uniform (defined herein as having a variation of less than 25% from a mean value) concentration of the antimicrobial agent can be established over a time interval between approximately 1 to 3 hours, as evidence by the simulations presented below. The susceptibility to the antimicrobial agent is determined by monitoring the growth of microbial cells dispensed onto the surface of the subregion, e.g. via a microscope, with reflected illumination equipped with the BF objective of (e.g. infinite plan objective 5×/0.12/∞/−(BF) or 10×/0.25/∞/−(BF/DF)). Since the cell-cell interaction on the solid phase is not expected to influence the minimum inhibitory concentration (MIC), cell numbers as high as 1000 CFU within one subregion, or higher, may be employed. On the other hand, cell numbers as low as 10 CFU, or lower, may be sufficient to avoid or sufficiently reduce the statistical possibility of having no cells dispensed into and thus retained upon the surface of a given subregion (when aliquots of a cell suspension are dispensed to multiple subregions, each having a different antimicrobial agent concentration).

An example non-limiting annular LD-AST device is presented in FIG. 18A. In the present disclosure, a device that facilitates the exposure of microbial cells from an aliquot of microbial cell suspension (e.g. a single droplet) within a single subregion is referred to as an LD-AST unit. The example LD-AST unit shown in the figure includes a gel-based growth medium configured to support the growth of microbial cells, where the gel-based growth medium is confined within a microwell having a wall 223 and bottom 224.

An antimicrobial agent is provided on and/or impregnated into a contact surface of the annular disk 220 (the lower surface of the annular disk 220). The annular disk 200 is adhered or attached (mechanically coupled) to a guiding ring 222. Upon the placement of these components onto a solid phase growth medium, an guiding well is formed by the wall of the guiding ring and the surface of the subregion of the surface of a solid phase growth medium 221 that is surrounded by the guiding ring 222. The antimicrobial agent diffuses inwardly from the annular disk 220. The upper surface of the guiding ring 222 may be hydrophobic such that upon the dispensing of an aliquot microbial cell suspension, the aliquot is guided, through the aperture, onto the exposed subregion of the solid phase growth medium. As shown in the figure, an upper surface of the guiding ring may have a beveled (e.g. curved) section 252 that is sloped toward the aperture for promoting the delivery of the liquid (wicking) to the surface of the subregion of the gel that is surrounded by the annular disk.

In some example embodiments, the number of microbial cells within the volume of the microbial cell suspension deposited onto the surface of the subregion may be less than 50, less than 20, or less than 10 cells. In some example embodiments, the volume of the microbial cell suspension deposited onto the surface of the subregion is less than 5 microliters, or less than 2 microliters.

In some example embodiments, the solid support may be contacted with solid phase growth medium that is provided in a microwell, such that the volume of the solid phase growth medium is less than 300 μl, less than 200 μl, less than 150 μl, less than 100 μl, less than 75 μl, or less than 50 μl.

As shown in the figure, the lower surface of the guiding ring 222may have a flashing feature 251 disposed adjacent to the aperture such that after coupling the annular disk and the guiding ring, flashing feature 251 penetrates the surface of the solid phase growth medium (e.g. penetrating to a depth of less than 500 microns, less than 250 microns, or less than 100 microns) for anchoring the assembly and preventing the influx of liquid underneath the disk, between the disk contact surface and the surface of the solid phase growth medium. In an alternative embodiment, the annular disk and the guiding ring may be formed as a monolithic component and the antimicrobial may be coated on and/or impregnated beneath the lower portion of the structure.

In order to demonstrate the capability of the guiding ring to assist in the delivery of the cell suspension to the exposed surface region of the solid phase growth medium surrounded by the annular disk, an experiment was performed as follows. A suspension of E. faecium with a concentration of 10⁵ CFU/mL was prepared. On a polyether-based thermoplastic polyurethane (TPU) film, having a thickness of 100 μm and diameter of 5 mm, two disks with diameters of respectively 1 and 0.8 mm were cut. The rings were placed on an agar gel. 1 μL of cell suspension was then dispensed on the rings and also on an uncovered part of the gel. The plate was incubated for 4 hours and the areas at which the sample had dispensed were imaged by 5× objective of a metallurgical microscope. The images are presented in FIG. 18J. In the case of no restriction (top image), the sample had expanded to a circular region of about 5 mm in diameter before drying. In the case of rings, the microbial cells have been guided into the inner region of the ring. The concentration of the cells is evident by the observation that the microcolonies are closely spaced.

As shown in the example embodiment shown in FIG. 18A, the annular disk has two characteristic radii; the inner radius r₁ and the outer radius r_{ad}. Between these two radii, the antimicrobial agent is coated on and/or impregnated beneath the lower contact surface of the disk. In some example embodiments r₁ lies within the range of 0.5 to 2 mm, or within the range of 0.8 to 2 mm, or within the range of 1 to 1.5 mm. In some example embodiments, r_{ad} is in range of 2 mm to 6 mm, or 2.5 mm to 4 mm.

While FIG. 17B and FIG. 18A show an example configuration of a solid support in the form of an annular disk (and annular guiding ring), it will be understood that this configuration provides but one example of a suitable structure for performing an LD-AST assay. Other example embodiments are provided below, in which a solid support is provided, the solid support at least partially surrounding an aperture (i.e. partially surrounding a central open region), where the solid support includes a contact surface having a chemical agent provided thereon and/or impregnated therebeneath, where the contact surface can be contacted with a solid phase growth medium such that subregion of the solid phase growth medium is accessible through the aperture, and such that at least a portion of the chemical agent diffuses laterally inwardly into the subregion, such that microbial cells deposited on a surface of the subregion are exposed to the chemical agent that has diffused below the surface of the subregion.

For example, while the preceding example embodiment employs an annular disk, it will be understood that the solid support that is employed to diffusively deliver the antimicrobial agent to the subregion may take on a wide variety of shapes, such as elliptical, square, or other shapes.

Furthermore, although the solid support shown in FIG. 18A fully encloses the subregion, in other example embodiments, the solid support may only partially enclose the subregion. For example, the solid support 220 may be provided in the form of two or more segments that partially surround (or enclose) an a gap or aperture or inner region 221, as shown in FIG. 18B, such that when the contact surface contacts the solid phase growth medium, antimicrobial agent diffuses inwardly from the contact surface into a subregion of the solid phase growth medium from multiple directions. For example, the solid support, having the antimicrobial agent provided thereon and/or therein, may contact the solid phase growth medium at a plurality of regions and diffusively deliver antimicrobial agent into the a subregion of the solid phase growth medium, such that when a first plane and a second plane are defined that each reside perpendicular to the surface of the solid phase growth media and each pass through the subregion, with the first plane being perpendicular to the second plane, the plurality of regions reside of both sides of each of the first plane and the second plane.

In some example embodiments, the antimicrobial agent may be uniformly distributed on and/or beneath the contact surface of the solid support. However, in other example embodiments, the antimicrobial agent may be provided at two or more separated regions on the contact surface.

In other example embodiments, a local area or subsurface density of the antimicrobial agent may spatially vary along the contact surface. For example, the antimicrobial agent may be provided on the contact surface according to an area density gradient or a subsurface density gradient. The area density gradient or subsurface density gradient may be provided such that an area density or subsurface density of the chemical agent is lowest in a surface region that is closest to the aperture, which, as shown in example simulations below, can be beneficial in generating a concentration within the subregion that exhibits a smaller time-dependent variation than a solid support having a uniform density of antimicrobial agent.

While many of the example embodiments of the present disclosure employ an LD-AST device in which the contact surface is configured to contact a top surface of a solid phase growth medium and diffusively deliver antimicrobial agent into the subregion, the solid support may also include a lateral confinement component that is configured to be immersed (submerged) into the solid phase growth medium. An example of such an embodiment is illustrated in FIGS. 18C-18G. FIGS. 18D and 18E show an example LD-AST device in which the solid support 220 includes a lateral cylindrical confinement component 225 that is submerged into the solid phase growth medium 250 when the contact surface 240 is contacted with the upper surface of the solid phase growth medium. The lateral cylindrical confinement component 225 is located further from the aperture than the planar contact surface 240, thereby presenting at least a partial outer barrier to the diffusion of antimicrobial agent, such that outward diffusion of the antimicrobial agent beyond the outer radius of the contact surface is at least partially restricted. Such an embodiment facilitates a more rapid buildup of concentration of the antimicrobial agent, and also facilitates the establishment of a concentration that has less temporal variation after a time frame of 1-2 hours. In some example implementations, the lateral conferment component may be configured to enclose a region having a width that is less than 5 mm, less than 4 mm, or less than 2 mm.

As shown in FIG. 18E, the lateral conferment component 225 can be employed to form a “virtual microwell” within a solid phase growth medium that extends beyond the outer radius of the solid support. In some example embodiments, the distal end of the lateral confinement member may contact the lower support surface on which the solid phase growth medium resides, thereby full enclosing a region of the solid phase growth medium. In another example implementation that is illustrated in FIGS. 18F and 18G, the solid support 220 having a lateral confinement component 225 may be contacted with a solid phase growth medium residing within a microwell 260. In such an example embodiment, the lateral confinement component may assist in maintaining a parallel orientation of the contact surface as the contact surface is brought into contact with the solid phase growth medium.

In some example embodiment, the contact surface may include a surface region, henceforth referred to as a “lateral contact surface”, which has antimicrobial agent provided thereon or immersed therein, where the surface region is configured to be immersed within the solid phase growth medium when the solid support is contacted with the solid phase growth medium for performing LD-AST. For example, with reference to FIG. 18D, the inner surface 226 of the lateral confinement member 225 may have antimicrobial agent provided thereon or immersed therein, such that the inner surface 226 forms at least a portion of the contact surface. Such a lateral contact surface may be inserted into the solid phase growth medium, for example, to a depth of at least 1 mm or at least 2 mm.

In some example embodiments, the solid support includes a tubular component, and where at least a distal surface region of an inner surface of the tubular component is coated with and/or impregnated with the antimicrobial agent, and where the tubular component is contacted with the solid phase growth medium such that at least a portion of the distal surface region is submerged within the solid phase growth medium, and such that the chemical agent diffuses inwardly within the subregion of the solid phase growth medium that resides within a lumen of the tubular component. An example of such an embodiment is illustrated in FIG. 18H, which shows a cross-section of an example cylindrical tubular component having a proximal region 270, a distal region 275, and an antimicrobial agent provided on and/or immersed within an inner surface 280 of the distal region 275. As shown in FIG. 181, the tubular component may be inserted into the solid phase growth medium such that a proximal portion of the tubular component extends outwardly from the solid phase growth medium. The microbial cell suspension 290 may be dispensed into the proximal portion 270 of the tubular component, where it is retained and contacted with the surface of the solid phase growth medium. The tubular component may be inserted such that a distal end of the tubular component contacts the lower support surface 295 that supports the solid phase growth medium, thereby enclosing the subregion and confining diffusion of the chemical agent within the tubular component. In one example implementation, the lower support surface 295 includes one or more mating features provided therein or thereon, the one or more mating features contact the distal end of the tubular component. The one or more mating features may include one or both of a projection and a recess, and may fully surround the distal end of the tubular component. A wall thickness of a distal portion of the tubular component may be less than 500 microns in order to facilitate the introduction of the tubular component into the solid phase growth medium.

The portion of the solid support on which the antimicrobial agent is provided, and/or within which the antimicrobial agent is impregnated, can be formed from a wide range of materials, including, but not limited to plastic materials such as polycarbonate, polypropylene, polysulfone and cyclic olefin copolymer that are either coated to render them more hydrophobic or hydrophilic, and porous materials such as paper and other porous material formed from, for example, cellulose esters, polyethersulfone, nylon, polycarbonate, polyester, polytetrafluoroethylene or polyvinylidene difluoride, either having hydrophobic or hydrophilic affinity for water.

In some example embodiments, a plurality for LD-AST units may be employed, with each LD-AST unit being configured to expose microbial cells retained thereon to a different antimicrobial agent. A determination of which microwells support microbial cell growth and which microwells inhibit microbial cell growth enables a determination of a minimum inhibitory concentration (MIC) of each antimicrobial agent. For example, as shown in FIG. 19A, a plurality of connected (mechanically attached or coupled) LD-AST units may be provided in in the form of a strip. The annular disks impregnated with an antimicrobial agent at different concentration levels (C₁ to Cₙ) are supplied with guiding (interaction) rings and assembled in the form of the strip 300. The number of concentration levels, n, may be in the range 2 to 7 or more and the concentrations may, in some implementations, double from one annular disk to the next along the array. As shown in FIG. 19B, a complementary strip, 310, may be provided having a plurality of microwells, each microwell containing a volume of agar gel based solid phase growth media (which may which are sealed by seal 311). When an LD-AST assay is to be performed, the two components may be brought into contact, as shown in FIG. 19C, such that the respective contact surfaces of the LD-AST units are contacted with solid phase growth media in respective microwells and the antimicrobial agent is diffused from the respective contact surfaces into the respective subregions of the solid phase growth media in the microwells. Aliquots of a microbial cell suspension, shown as droplet 320, may then be dispensed inside the interaction rings, such that microbial cells with the aliquots are retained on the respective subregion surfaces, where they are exposed to the antimicrobial agent that has diffused into the subregions. Multiple arrays may be employed to assess multiple antimicrobial agents at varying concentrations, with the LD-AST units and the solid phase growth media microwells optionally formed as respective two-dimensional arrays (e.g. a monolithic array in the form of a plate).

FIGS. 19D and 19E illustrate an example multi-unit array embodiment involving the LD-AST unit shown in FIG. 18B. In some example embodiments, one or more of the array of LD-AST units and the array of solid phase growth medium microwells include a keyed feature that facilitates alignment between the respective contact surfaces and the respective microwells. The keyed feature may facilitate alignment of one or more of a lateral position and a depth of each contact surface relative to the respective microwells.

FIG. 22 provides a flow chart illustrating an example method for performing LD-AST. The method comprises of preparing a cell suspension having a sufficiently known classification and concentration at step 100. The classification should be known to the extent that a panel of antimicrobial can be decided at step 601. An example of classification granularity is bacterial/fungal and gram positive/negative.

The concentration should be known to the extent that a cell number of between 10 and 1000 in the region of interest is ensured after sample dispensing. Once the antimicrobial panel is selected, in step 602, plurality of drug exposure regions are formed by overlaying appropriately shaped drug impregnated discs on the gel. The antimicrobial agent diffused inwardly into the subregion of the solid phase growth medium and to establish an antimicrobial agent concentration across the drug exposure regions (step 603). In step 604, aliquots of cell suspension are dispensed on the drug impregnated discs, which are may be supplied with form and structure for guiding the dispensed sample into the drug exposure region. In step 605, the growth behavior of the microbial cells in each drug exposure region is monitored by the time lapse microscopic imaging. Comparing the growth rate of microbial cells in each drug exposure region with the growth rate of cells in a drug free region (control) the MIC value for each antimicrobial in the panel is determined in step 606. These values, in association with the identification of the cells, during steps 607 to 609, are used to determine the antimicrobial susceptibility profile of the cells in step 610.

The present example method is performed based on a cell suspension that has been sufficiently characterized in terms of an estimated cell concentration and at least a presumptive initial microbial cell class determination (e.g. at least a determination of bacterial vs. fungal cells, and a determination of Gram status for bacterial cells). The cell class is employed to selecting an appropriate antimicrobial agent test panel (e.g. a Gram-positive or Gram-negative panel).

The cell concentration need not be accurately known and may be employed merely to confirm that after aliquoting, each aliquot is expected to contain a sufficient quantity of microbial cells to avoid the possibility of dispensing an aliquot contains too many microbial cells to facilitate growth monitoring, and to avoid the possibility that the aliquot is absent of microbial cells due to statistical fluctuations. For example, the example case of a testing a Gram-positive bacterium for determining its susceptibility to a panel of N_{d} drugs, each having N_{c} concentration levels is considered. After dispensing ~1 μL of the suspension in N_{d}×N_{c} LD-AST units (N_{d}=Number of drugs, N_{c}=Number of concentration), each LD-AST unit receives C_{c} (CFU per microliter) cells on its ROI of ~1 mm². As a result, the area around each microbial cell, σ, is 106/Cc μm². In the case of a cell suspension with 10⁶ CFU/mL=10³ CFU/UL, σ=10³ μm². This corresponds to a surface coverage of merely 0.1%, meaning that each cell can be considered as proliferating independent of its neighbors. On the other hand, cell numbers as low as 10 CFU on each ROI, corresponding to a cell concentration of 10⁴ CFU/mL, can be comfortably monitored without being impacted by the consequences of Poisson statistics. The initial cell concentration of the microbial cell suspension may therefore be assessed to confirm that it lies within this range.

The cell suspension may be prepared according to a wide variety of methods and may be obtained, either directly or indirectly (with or without a preceding growth step), from a wide range of sample types. In one example embodiment, the microbial cell suspension may be obtained by harvesting microbial cells from a microcolony, as per the example methods described above, for example, after the microcolony has grown to a target size and has optionally been presumptively classified (e.g. as bacterial vs. fungal, an optionally Gram-positive vs, Gram-negative). The harvested microcolony may be resuspended into an appropriate medium (for example, saline solution, or growth media such as, for example, TSB or BHI) and optionally diluted or concentrated, and then dispensed onto the LD-AST units. In another example, the microbial cells may be obtained by processing a blood culture sample to obtain a microbial cell suspension, for example, according to the methods disclosed in International Patent Application No. PCT/CA2019/050716.

The presence of growth or non-growth may be monitored by microscopy techniques or other methods which can monitor or determine the temporal change in microbial cell count at a region with an accuracy of approximately 2-fold. In one example embodiment, the drug exposure regions may be intermittently (e.g. once every 30 minutes) imaged by a microscope, such as a microscope equipped with 5× or 10× objective and the sequence of images may be compared employing image processing methods to verify whether or not the cells are growing and/or proliferating. The present inventors have found that the halting of microbial growth due to the effect of an antimicrobial agent at MIC concentration is typically detected between 3 to 5 hours of incubation.

In order to illustrate the effect of the dimensions of the example LD-AST unit shown in FIG. 18 on the performance of an LD-AST assay, concentration profiles were calculated for the selected diffusion scenarios for 5 minutes, 30 minutes, 1 hour, 2 hours, 3 hours and 4 hours based on the simulation of the diffusion equation (Fourier's equation) by the finite difference method (a time march). The numerical scheme used was a first order upwind in time and a second order central difference in space. The diffusion equation to be solved in the context of the present disclosure was written as δC/δt=D(δ2C/δz2+(1/r) δ(r δ(C/δr)/δr), where C(r,z,t) is the concentration which depends on cylindrical coordinates, r and z, but not the polar angle ⊖. The coefficient D is the diffusion coefficient and δ/δt, δ/δr, and δ/δz are respectively partial derivatives with respect to time (t), and spatial coordinates, r and z.

Spatial profiles of the antimicrobial concentration were plotted, at various times after initial contact, along a line passing through the center of the region of interest in FIGS. 20A to 20H. The plots in FIG. 20A corresponds to a case of r₁=1.5 mm, r_{ad}=3 mm, r₂=14 mm, h=4 mm, and a uniform antimicrobial agent distribution between r₁ and r_{ad}. As it is observed, within the first 30 min after placing the annular disk on the gel, the surface concentration of the antimicrobial agent across the subregion exposed beneath the aperture becomes uniform, however the concentration levels decrease over time.

In order to further illustrate this time-dependence of the concentration of the antimicrobial agent, FIG. 21A plots the level of antimicrobial agent concentration at the center of subregion for different combinations of r₂, h (with r₁ and r_{ad} fixed respectively at 1.5 mm and 3 mm except for the case of dual 2 where r_{ad}=4 mm) and initial antimicrobial agent distribution between r₁ and r_{ad}. Moreover, the change in this antimicrobial agent concentration during the period of reaching max concentration at ~0.5h and 4 h after placing the annular disk on the gel is presented in FIG. 21B. As it is observed, the variation over the period of ~0.5 h and 4 h is about 160%. This strong dependence of concentration on time may be undesirable because it complicates the quantification of AST. The present inventors thus sought configurations that achieved smaller temporal variations of the concentration across the subregion. In one simulation study, the inventors modified three parameters, namely r_{ad}, r₂, and h.

The plots in FIG. 20B correspond to a case of r₁=1.5 mm, r_{ad}=3 mm, r₂=14 mm, h=2 mm, and a uniform antimicrobial agent distribution between r₁ and r_{ad}.

These plots are qualitatively comparable with the plots of FIG. 20A. However, referring to FIGS. 21A and 21B, a subtle difference is observed; and the variation in antimicrobial agent concentration is 128% instead of 160% of FIG. 20A. Accordingly, decreasing the gel thickness appears to achieve an improved performance, albeit to an insignificant extent. On the other hand, a thinner gel is more prone to gel dehydration during the assay, in particular for cases that r₂ is much larger than r_{ad} and significant fraction of the gel surface is exposed to the ambient environment.

The beneficial effect of increased lateral confinement of the gel is illustrated in FIG. 20C. In this case there is a reduction in the magnitude of the concentration variation from the peak value of 160% (corresponding to FIG. 20A) to 132%. Though reducing either r₂ or h had minor benefit in lowering the temporal variation of the concentration, simultaneously applying both changes results in 76% corresponding to the plot of FIG. 20D, which is a significant improvement over the less restricted case of FIG. 20A.

Further improvement in assay performance can be achieved by employing annular rings which have been impregnated in a radially variable manner. In order to illustrate this, the concentration profile was calculated for the example cases of two dual-annular rings, whose intensity profile were respectively as the following:
Dual1: C₀=0.5, if 1.5<r<2.5; C₀=1, 2.5<r<3; C₀=0, otherwise (see FIG. 20E).
Dual2: C₀=0.5, if 1.5<r<3; C₀=1, 3<r<4; C₀=0, otherwise (see FIG. 20G).

The resulting profiles are respectively presented in FIGS. 20F and 20H. As can be seen from FIG. 21B, the variation in concentration at the center over the relevant time has dropped, respectively, to 61% and 19%. This indicates that depositing the drug with a radially varying concentration, in the form: {C₀ provided with an increasing function in r₁<r<r_{ad}, C₀=0 otherwise} can significantly decrease the concentration variation across the ROI over the relevant time period.

The transient concentration variation and the potential concentration non-uniformity of the diffused antimicrobial agent across the ROI can be reduced by preparing the annular disks with radially increasing dried antibiotic concentration. One example implementation of this approach has been described in Example 9B. In order to illustrate the implementation of the method and its improved performance, the following experiments were performed. The antibiotic solution was replaced with a dye solution and employed to prepare an annular disk. In FIG. 21C, the disk is shown after applying water and drying (top-left) and after punching the hole inside (top-right). The relative concentration of the dye is presented in the plot at the bottom of the figure. As it is observed, a concentration gradient has been created such that the concentration is increasing by approaching the outer edge. This example indeed offers a practical method for adjusting the concentration profile.

In order to illustrate the feasibility of the LD-AST method, several experimental LD-AST assays were performed over different combinations of microbial species and antimicrobial agent.

In one case, an LD-AST test strip was fabricated following the method of Examples 9A and 10, coating different concentrations of Norfloxacin on the annular disks in 0-16 μg range. Three types of strips were prepared: low volume agar type with 80 μL of gel per microwell, medium volume agar type with 150 μL of gel per microwell, and high-volume agar type with 350 μL of gel per microwell. The corresponding gel thickness was respectively 2 mm, 4 mm, and 9 mm. The annular disks were then placed on the gels. The images of the microwell strip, for the case of low volume agar type, is presented in FIGS. 23A and 23B, where 220, 221, 222, and 223 respectively, refer to the drug impregnated annular disc, the drug exposure region, the guiding ring, and the gel. 1 μL of Escherichia coli cell suspension, which had been prepared according to the method of Example 12, was dispensed into the ROI of each LD-AST unit. The strips were incubated at 37° C. and their ROI were imaged by an epi-illuminated microscope having a 5× objective once every hour. These images are presented in FIGS. 24A-24C (low volume gel), 25A-C (medium volume gel), and 26A-C (high volume gel) for incubation times of t=3hours, t=4hours and t=overnight, respectively.

The labels in FIGS. 24A-24C show the mass of the of the antimicrobial agent that was coated on the annular disk during preparation. As shown in FIG. 21A, the resulting concentration that is generated within the solid phase growth medium varies as a function of time, initially rising to a peak and then decaying to a plateau. Despite this time variation, an effective antimicrobial agent concentration may be associated with each microwell. This effective antimicrobial agent concentration may be estimated, for example, by modeling the diffusion of the antimicrobial agent, as in FIG. 21A, or, for example, by comparing the results of the LD-AST assay to a reference assay such as broth microdilution assay.

In the case of low volume gels (FIG. 24A-24C), since the antimicrobial agent is expected rapidly and efficiently diffuse throughout the volume of the solid phase growth medium, and since it is expected that the antimicrobial agent diffuses nearly completely from the annular disk, the effective antimicrobial agent concentration may be estimated by dividing the mass of the antimicrobial agent that was provided on the annular disk by the volume of the gel. As can be seen in FIG. 21A, this concentration is expected to be a good approximation to the true concentration during a time window beyond 1-2 hours. Accordingly, the effective antimicrobial agent concentration for the case low-volume gels of FIGS. 24A-24C was estimated to be 0.25 times of the drug mass that was impregnated on the disk. For instance, the drug concentration at the drug exposure region of the annular disk loaded by 16 μg of Norfloxacin is 4 μg/mL. Accordingly, the drug concentrations at different drug exposure region is calculated and presented in FIG. 24C.

Comparing FIGS. 24B and 24C, one can observe (or computationally determine) that for the LD-AST unit labeled by 0.5 μg (having an estimated effective concentration of 0.1 μg/ml), there appears to be a lack of visible growth between 3 and 4 hours of incubation. This halting in growth was verified by imaging the ROIs after overnight culture, as illustrated in FIG. 24C. As a result, the MIC value for this test was estimated as 0.1 μg/mL. According to the interpretation standards published by the Clinical and Laboratory Standards Institute (CLSI) in the USA and the European Committee on Antimicrobial Susceptibility Testing (EUCAST) in Europe, this MIC value can be classified as S=Susceptible. This value can be compared to the MIC value of 0.1 acquired via a conventional microdilution methodology and the value <=0.5 S μg/mL reported by Vitek 2 (Biomerieux). Thus, the MIC value of 0.1 is in essential agreement with both reference methods, since the reported MIC is within ±1 doubling dilution from the reference method.

FIG. 25A-25C and 26A-26C, respectively, correspond to intermediate volume (gel volume of 150 μL in microwell) and high volume (gel volume of 350 μL in microwell) cases. In these cases, due to the larger temporal variation in concentration during incubation, an effective antimicrobial agent concentration may be determined by correlating the observed results with a reference microdilution assay. As can be seen in the figures, the halting of microbial growth at the microwell labeled with 1 μg is evident.

The present inventors have found that in the cases of lower gel volumes, the lower time dependence of the antimicrobial agent concentration, when compared to larger gel volumes, results in greater clarity in determining which microwell corresponds to growth inhibition. This is illustrated by presenting the case of determining MIC values for Staphylococcus aureus exposed to Vancomycin. The images of ROIs for the two types of LD-AST unit, i.e. “thin type” and “mid-thick type”, are presented in FIGS. 27A and 27B, respectively. Even though there is a distinct difference between the colonies of the LD-AST unit at MIC and the next well (with lower drug level) for the case of “thin type” (FIG. 27A), the corresponding difference is not pronounced for the “mid-thick type” (FIG. 27B). In this case, the cell proliferation at the borderline LD-AST unit, such as the one labeled 12 μg, would be preferably determined by image processing. For example, time-lapse microscopic images can be acquired are aligned and difference images (obtained via subtraction of aligned images) may be processed to detect evidence of growth. For instance, this procedure has been performed according to Example 13 on the image with 12 μg in FIG. 27B. The difference image, which is presented at the third row, is not null, indicating that the colonies had indeed continued to grow. This conclusion was verified by further incubating the strips for the overnight.

From FIG. 27A, the MIC value is found to be 3 g/mL. According to the interpretation standards published by CLSI, the MIC value acquired can be classified as I=Intermediate. While being under the “minor error” with the reference value, the acquired MIC is clearly larger than the value of 1 μg/mL (Susceptible; S) from Vitek 2. Moreover, broth microdilution AST was performed according to Example 11 and an MIC of 1.5 μg/mL was found, which correspond to an “S” result according to CLSI. While being not in the categorical agreement, MIC of the test method (LD-AST) is +/−1 doubling dilution from the reference method, which corresponds to a minor error in comparison with the broth microdilution AST.

The minor difference between the MIC of LD-AST and reference methods is expected. According to the standardized protocol for disk diffusion [HARDYDISK™ ANTIMICROBIAL SENSITIVITY TEST(AST)—Instruction for use], Staphylococcus or Enterococcus spp. require 24 hours of incubation for vancomycin and oxacillin, compared to the conventional 16 to 18 hours for the other organisms and agents. According to the literature, vancomycin MIC distributions by tube microdilution and agar testing are markedly different from those evaluated by broth microdilution and might differ for 24 hours and 48 hours [Vaudaux, Pierre et al. “Underestimation of vancomycin and teicoplanin MICs by broth microdilution leads to underdetection of glycopeptide-intermediate isolates of Staphylococcus aureus.” Antimicrobial agents and chemotherapy vol. 54,9 (2010): 3861-70. doi: 10.1128/AAC.00269-10]. Moreover, Vancomycin MICs generated by E-test (bioMérieux AB BIODISK, bioMerieux, Inc., Hazelwood, Mo.)) are known to be higher than MICs determined by broth or agar dilution. Thus, experimental results with vancomycin were cross-validated with both broth microdilution and simplified population analysis agar [Determination of minimum inhibitory concentrations. Andrews J M J Antimicrob Chemother. 2001 July; 48 Suppl 1( ):5-16.] method. As noted above, the observed results can be compared with a reference method to infer a suitable effective antimicrobial agent concentration.

In order to verify the observed difference between the MIC for the low-volume gel and the typical MIC values reported by EUCAST for S. aureus and Vancomycin was not caused by the geometry of the annular disk and its corresponding diffusion dynamics, the following experiment was performed. A two-fold serial dilution of the target antimicrobial agent was prepared in water. Then 20 μL of each dilution was pipetted onto the surface of “thin-type” microwells that were prepared according to the method of Example 10. After about 10 minutes, the antimicrobial agent solution had diffused into top of the gel. 1 μL of a microbial cell suspension was then added and together they were incubated at 37° C. until the microbial colonies at the control well (zero antimicrobial agent concentration) were visible. The MIC found in this manner was 3 μg/mL.

In order to further illustrate the insensitivity of the LD-AST to variations of the antimicrobial agent concentration at the ROIs during the first hour of the assay, the assay was performed in two different protocols; a first direct protocol involving immediate inoculation of the microbial cell suspension after contacting the annular disks with the solid phase growth medium, and a second delayed protocol in which the microbial cell suspension was inoculated 1 hour after contacting the annular disks with the solid phase growth medium. The result is presented for the case of Staphylococcus aureus exposed to Vancomycin in FIG. 28. As can be seen in the figures, there is no observable differences in microbial cell growth profiles for the two protocols, and the MIC is equal for the two protocols.

### Performing Antimicrobial Susceptibility Testing Against Slow Pathogenic Fungal Cells

Pathogenic fungal cells are slow growing and commercially available antimicrobial susceptibility testing for fungal cells is correspondingly slow. In the present example, it is illustrated that the LD-AST methods and devices, in terms of time to result, are similar for bacterial and fungal cells. Moreover, since the number of available antifungal agents is smaller than the number of antibacterial agents, a fungal microcolony may be resuspended in a lower volume, for example 20 μL for fungal cells versus 100 μL in the case of bacterial cells. Accordingly, the fungal cell harvesting can be performed when the cell number reaches ~500 CFU (~9 cycles of growth).

A series of LD-AST units having different levels of Amphotoricin B were prepared and tested against Candida albicans (ATCC 90028). The resulting images of ROIs at incubation times of 3 and 4 hours are presented in FIG. 29. The images were analyzed following the procedure of Example 13 and it was found that the cells at the LD-AST unit corresponding to 2 μg/mL had stopped growing. Therefore, the MIC was determined to be 2 μg/mL. The conclusion of growth at the LD-AST unit corresponding to 1 μg/mL, though not easily noticed in FIG. 29 by visual inspection of t=3 and 4 incubation images, was verified by referring to the overnight incubation row in the figure.

### Performing Antimicrobial Susceptibility Testing on Positive Culture Samples

Positive culture samples, such as positive blood culture samples, can be tested for the antimicrobial susceptibility employing methods described above with minimum or no additional sample processing steps. This flexibility is due to two features: i) insensitivity of the method to the microbial cell concentration at least over range spanning two orders of magnitude, and ii) performing assay on solid phase and monitoring cell growth via imaging microcolonies. These features imply that exact measurement of cell concentration, typically via optical scattering measurement requiring low level of background scatterers, is not required. In order to demonstrate the flexibility of LD-AST for performing AST on positive blood culture samples the following experiment was performed.

Methicillin-resistant Staphylococcus aureus (MRSA 111 with not strong resistance) was spiked into 10 mL of whole blood samples at the nominal concentration of 5-10 CFU/ml. Then, an FA Plus aerobic blood culture bottles were inoculated with 10 mL of spiked whole blood and incubated at 37° C. until the culture turns positive. At this time, 30 μL of glycerol stock of the bacterial cell was inoculated in 3 mL of TSB and incubated at 37° C. for 3 hr with shaking at 150 rpm. Based on OD measurements, serial dilutions of the respective bacteria were prepared in TSB at a nominal concentration of 10⁵ CFU/mL. An aliquot of the positive blood sample was diluted in TSB by 1000-fold. The two samples were simultaneously tested for susceptibility against Oxacillin with “thin strip” type LD-AST. The results, which are presented in FIG. 30, indicate similar MIC value of 1 μg/mL.

Performing Antimicrobial Susceptibility Testing on a Panel of Antimicrobials In this example, a panel of antimicrobial agents similar to the Sensititre Gram Positive GPALL1F Plate (ThermoFisher Scientific) was prepared. The commercial plate, which is in the form of a 96 well microplate, includes 23 antimicrobial agents, each at several clinically-relevant concentrations. The microbroth dilution AST testing was performed against MRSA-110 following the protocol of example 14, and the result was presented in FIG. 31A.

Thin-type LD-AST units were prepared corresponding to the plate above, i.e. the same antimicrobials and the same concentrations were employed, such that there was a one-to-one correspondence between microbroth dilution well and LD-AST unit. The LD-AST units were placed on the gel wells and the LD-AST was performed according to the method of example 15. The result was determined and presented in FIG. 31B. As it is seen the results are concordant with the result of commercial broth dilution AST.

### LD-AST for Several Example Antimicrobial Agent-Microbial Cell Combinations

The present example is provided to illustrate the potential similarity in performance between the LD-AST method and broth microdilution AST, even though the time to result for LD-AST is merely 4 hours, in stark contrast to the 16-20 hours needed for conventional broth microdilution AST.

Thin type LD-AST units were prepared corresponding to a selected panel of antimicrobial agents, and the LD-AST method was performed on selected microbial strains according to the method of example 15. The results are presented in FIG. 32, in which abbreviations are employed according to the CLSI guideline as the following: EA-MIC of the test method is +/−1 doubling dilution from the reference method, CA/minor category agreement (i.e., susceptible(S), intermediate (I) or resistant (R) interpretation of the MIC for the test method matches the S, I or R interpretation of the reference method).

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1: Microbial Cell Culture Preparation

Gram-positive bacteria except Staphylococcus aureus (SA) and Streptococcus pneumoniae (SP) cell culture was prepared as follows:
1. Thirty μL of respective bacteria species and strain glycerol stock was inoculated in 3 mL of tryptic soy broth (TSB) and incubated at 37° C. for overnight with shaking at 150 rpm.
2. Tenfold diluted culture in TSB was incubated at 37° C. for 1 hour (Enterococcus faecalis (Efcl), Entercoccus faecium (Efcm) and Streptococcus agalactia (Sag)) or for 2 hours (Straphylococcus epidermidis (SE), Staphylococcus haemolyticus (SH) and Streptococcus pyogenes (Spyo)).

Gram-negative bacteria except Pseudomonas aeruginosa (pa) cell culture was prepared as follows:
1. Thirty μL of respective bacteria species and strain glycerol stock was inoculated in 3 mL of TSB and incubated at 37° C. for overnight with shaking at 150 rpm.
2. Tenfold diluted culture in TSB was incubated at 37° C. for 1 hour (Acinetobacter baumannii (AB), Enterobacter cloacae complex (Ecl), Enterobacter aerogenes (EA), Escherichia coli (EC), Klebsiella oxytoca (KO), Klebsiella pneumoniae (KP) and Proteus mirabilis (PM)) or for 2 hours (Serratia marcescens (SM)).

Staphylococcus aureus (SA) cell culture was prepared as follows:
30 μL of respective strain glycerol stock was inoculated in 3 mL of TSB and incubated at 37° C. for 3 hours with shaking at 150 rpm.

Streptococcus pneumoniae (SP) cell culture was prepared as follows:
30 μL of respective species or strain glycerol stock was inoculated in 3 mL of TSB and incubated at 37° C. for 3 hours with shaking at 80 rpm in the presence of co₂ generating pouch.

Pseudomonas aeruginosa (PA) was prepared as follows:
1. Six μL of PA strain glycerol stock was streaked on tryptic soy agar (TSA) with 5% sheep blood plate and incubated at 37° C. for overnight (P1).
2. Bacteria colony was subcultured one more time on agar plate (P2).
3. One colony from the plate was inoculated in 3 ml.

Fungal cell cultures were prepared as follows:
1. Thirty μL of respective fungi species and strain glycerol stock was inoculated in 3 mL of TSB and incubated at 30° C. for overnight with shaking at 150 rpm.
2. Tenfold diluted culture in TSB was incubated at 30° C. for 2 hours (Candida albicans (CA), Candida glabrata (CG), Candida parapsilosis (CP) and Candida tropicalis (CT)).

Based on optical density (OD) measurements, serial dilutions of the respective bacteria were prepared in TSB at a nominal concentration of 10³ CFU/mL.

### Example 2: Preparation of Spiked Whole Blood Samples

Blood samples with volumes between 5-8 mL were drawn from healthy individuals into BD Vacutainer® with SPS tubes. The tubes were kept at room temperature prior to being spiked with bacterial cells for an average period of 4 hours. Then, 100 μL of bacterial cell suspension with nominal concentration of 10⁵ CFU/mL of respective bacterial cells was added to 4 mL of blood and mixed by gentle vortexing. Thus, the concentration of microbial cells is nominally about 2.5×10³ CFU/mL.

### Example 3: Preparation of Spiked Phosphate Buffer Samples

Bacterial cell suspension stock of 100 μL, having about 10⁵ CFU/mL of respective bacterial cells was added to 4 mL of 1 mM Phosphate Buffer (PB) and mixed by gentle vortexing. Thus, the concentration of microbial cells is nominally about 2.5×10³ CFU/mL.

### Example 4: Preparation of Blood Lysis Reagent

The blood lysis reagent solutions were prepared by combining 10 ml of a carbonate-bicarbonate buffer solution prepared with a buffer concentration of 100 mM pH of 10 with 10 ml of a solution having a concentration of 40 mg/ml of SPS, a saponin concentration of 70 mg/ml, and a Triton X-100 concentration of 0.3 w/v, to obtain reagent solutions having a volume of 20 ml, an SPS concentration of 20 mg/ml, saponin concentration of 35 mg/ml, a Triton X-100 concentration of 0.15% w/v, a buffer concentration of 50 mM, and pH values were in the range of 9.5-10.

### Example 5: Sample Treatment of 4 mL Spiked Whole Blood Samples

Sample preparation was performed for spiked whole blood samples as follows:
1. In a 15 mL centrifuge tube, 4 ml of blood lysis reagent was added to 4 ml of spiked whole blood sample.
2. The centrifuge tube was mixed by vortexing for 1 minute at maximum speed of the vortexer.
3. The centrifuge tube was centrifuged at 4000 rpm for 8 minutes.
4. A supernatant of 7.9 ml was removed.
5. The first wash cycle was performed, by adding 2.9 mL of wash buffer to the residue, mixing the solution was mixed by gently vortexing, centrifugation at 4000 rpm for 3 min, and withdrawing and discarded 2.9 mL of supernatant such that 100 μl of residual liquid was retained.
6. The second wash cycle was performed, by adding 2.9 mL of wash buffer to the residue, mixing the solution was mixed by gently vortexing, centrifugation at 4000 rpm for 3 min, and withdrawing and discarded 2.9 mL of supernatant such that 100 μl of residual liquid was retained.
7. The third wash cycle was performed, by adding 1.9 mL of wash buffer to the residue, mixing the solution was mixed by gently vortexing, centrifugation at 4000 rpm for 3 min, and withdrawing and discarded 1.9 mL of supernatant such that 100 μl of residual liquid was retained.
8. The fourth wash cycle was performed, by adding 1.9 mL of wash buffer to the residue, mixing the solution was mixed by gently vortexing, centrifugation at 4000 rpm for 3 min, and withdrawing and discarded 1.9 mL of supernatant such that 100 μl of residual liquid (cell suspension) was retained.

### Example 6: Preparation of the Agar Plates

Agar plates were prepared with final agar concentration of 1-5% w/v. To prepare Tryptic Soy Agar Blood (TSAB) plates, TSAB Dehydrated culture media: Formula per liter, Agar 13.5 gm, Casein Peptone 15.0 gm, Soy Peptone 5.0 gm, Sodium Chloride 5.0 gm (Hardy Diagnostics, CA) was used. To prepare agar plates with higher agar concentration (>1.35% w/v), Agar Bacteriological Grade dehydrated culture media (Hardy Diagnostics, CA) was used according to the desired agar concentration.

Sterilized defibrinated sheep blood (Hardy Diagnostics, CA) was used to enrich the gel and enhance the bacterial growth. The preparation steps were as the following:
1. TSAB powder with agar extra powder (if needed) was dissolved in water molecular biology grade on a hot plate at 100° C. inside a water bath for 10 mins.
2. The solution (with the water bath) was autoclaved for 15 mins at 121° C.
3. The solution was cooled down to around 55° C. for 15 minutes.
4. 3-5% sheep blood (pre-warmed in a water bath to 50° C. for 30 mins) was added to the cooled solution and mixed well.
5. The solution was dispensed to petri dishes (35×10 mm) and let solidified for 5 mins.
6. To prevent microbial contamination, plates were stored in a sterile environment.

### Example 7: Measuring the Growth Rate and Colony Size of a Microbial Cell on an Agar Plate

The growth rate of a microbial cell on an agar plate is determined through the following steps:
1. Prepare starting cell suspension with a nominal concentration of 10⁵ CFU/mL.
2. Dispense 1 μL of the cell suspension on one of three identifiable regions on an agar gel plate and allow them to spread over a mini-culture region (MCR) and air dry. Thus, there will be 3 MCR, identified as MCR1, MCR2, and MCR3, on the plate.
3. Image on the MCRs at t₀=0 hours.
4. Incubate the plate at 37° C. for 1 hours.
5. Image MCR 1 at time point of 2 hours for bacterial and 4 hours for fungal species.
6. Calculate the areas of the microcolonies by analyzing the images and calculate their corresponding diameters, D, through the relation D=2*sqrt(area/3.1416). Then calculate the average diameter by averaging over all microcolonies.
7. Remove the microbial content of the MCR by a swab and resuspend it in 200 μL of TSB growth media (cell resuspension).
8. Serially dilute the cell resuspension in TSB with multiples of 10, and label the resulting samples as S10⁰, S10⁻¹, S10⁻², S10⁻³, and S10⁻⁴.
9. Plate the samples and incubate them for overnight.
10. Repeat steps 5 to 9 for MCR 2 and MCR 3, respectively at 3, 4, and optionally 6 hours for bacterial species (5, and 6 hours for fungal species). Count the overnight colonies and tabulate them.
11. Accordingly, calculate the number of microbial cells on the respective MCR.
12. Determine the growth rate by calculating the slope of the cell number versus time plot on a logarithmic-linear plot.
13. Plot average colony diameter versus the colony cell content.
14. Determine the average diameter at which the cell number in a microcolony reaches 10³ and 10⁵.

### Example 8: Measuring the Recovery Rate of Separating a Microbial Cell From a Blood Sample and Having it to Form Colonies on an Agar Plate

Recovery rates were measured for spiked whole blood samples as follows:
1. In a cartridge, as shown in FIG. 13A and containing 4 mL of BLR of example 4 in chamber 503, four mL of spiked whole blood sample (prepared according to example 2) was added to chamber 501.
2. The blood sample and BLR were mixed by moving BLR to chamber 501 and moving the resulting mixture back and fore between chambers 501 and 503 for 5 times.
3. The cartridge was centrifuged at 3000 g for 8 minutes.
4. A supernatant of 7.9 ml was moved to the waste chamber 506.
5. The first wash cycle was performed, by adding 2.9 mL of wash buffer to the residue, mixing the solution by gently moving it between chambers 501 and 503.
6. Centrifuge at 3000 g for 3 min,
7. A supernatant of 2.9 ml was moved to the waste chamber 506.
8. Repeat steps 5 to 7 for second wash.
9. Remove the 100 μL residue (cell suspension)
10. Plate the cell suspension on an agar plate and incubate over night at 37° C.
11. Count the colonies and calculate the recovery with respect to the expected number according to control plate.

### Example 9A: Coating of Annular Disks With Antibiotic

Blank AST paper disks (Hardy Diagnostics, Z7121) that were 6.35 mm in diameter and 0.75 mm thick, were modified to include a 3 mm hole in the center.

Each paper disk has the capacity to absorb approximately 20 μL of liquid before being saturated. For each antibiotic, a two-fold serial dilution in water was made which included concentrations near its suspected MIC value. The disks were separated into individual wells of a 96-well microplate, and then 20 μL of each antibiotic dilution was pipetted over paper disks ensuring that the entire disk was evenly wetted. After incubation for about 20 minutes at room temperature, the disks in the microplate were dried in vacuum desiccator. Alternatively, individual disk separated in the microplate well could be soaked in an excess volume (30 to 50 μL) of the antibiotic dilution. After incubation, the excess fluid is removed by pipette and dried in a vacuum desiccator. The microplate was then covered with an adhesive cover and stored at 4° C.

### Example 9b: Coating Antibiotic on Annular Disks With Radially Varying Concentration

Blank AST paper disks (Hardy Diagnostics, Z7121) that were 6.35 mm in diameter and 0.75 mm thick, were separated into flat culture dishes (for example, a 24-well culture dish). A two-fold serial dilution of each antibiotic was made as described in Example 9A, and then 20 μL of each dilution was pipetted, dropwise onto each disk. The disks were then dried in a desiccator under high vacuum (0.5 to 2 m Torr) for 1 hour. Once dried, 15 μL of water was pipetted into the center of each disk such that water radiated outwards to the edge of the disks. The disks were then dried again under the vacuum desiccator for 1 hour. A 3 mm hole was then punched through the center of the disks. They were transferred to individual wells of a 96-well microplate which was then sealed with an adhesive cover and stored at 4° C.

### Example 10: Preparing Blood Agar Microwells

Tryptic soy agar blood base (Hardy Diagnostics C5221) was prepared with 1% defibrinated sheep's blood according to the manufacturer. While the agar was still warm (45 to 50° C.), it was pipetted into the wells of an 12×8-well strip plate. For “Thin-gel” case, approximately 50 μL of agar was dispensed into an individual well to give a gel thickness of ~1 mm. For the “mid-thick gel” case, approximately 150 μL of agar was dispensed to gel a gel thickness of ~3 mm. Finally, for the “thick gel” case, approximately 350 μL of agar was dispensed to gel a gel thickness of ~7 mm. The agar gels were then cooled to room temperature to solidify, and then either used immediately or stored at 4° C. with an adhesive cover over the wells to prevent gel drying.

### Example 11: Performing Broth Microdilution AST

Intermediate twofold dilutions of antimicrobial agents in broth (Tryptic soy, and in some cases Brain Heart Infusion). 100 μL of the antimicrobial agent/broth solutions were dispensed into each well of 96-well plates in triplicates. Then, to each well 10 μL of the 5×10⁶ CFU/mL cell suspension, which was prepared according to the method of example 5, was added. The plate was sealed and placed in the incubator at 35-37° C. for 16-24 hours for MIC value to be determined.

### Example 12: Inoculation Preparation

Inoculum was prepared using broth culture method. In brief, an aliquot, 30-100μl depending on the bacterium to be cultured, of bacterial glycerol stock was thawed and added to 2-3 mL of an appropriate media (tryptic soy or brain heart infusion). Bacterial culture was then incubated at 37° C. with a shaker set at 150 rpm and grown for 2-3 hrs. Within 15 minutes of the preparation, inoculum suspension was adjusted to 0.5 McFarland standard (1×10⁸ CFU/mL), and was subsequently diluted 1:20 in broth to yield 5×10⁶ CFU/mL.

### Example 13: Processing the Time-lapse Images for Determining Growth

To align imaging data acquired at different time points (2, 3, 4 and 5 hours after seeding), 2D-2D registration (only translation and rotation are permitted) with rigid transformation constrains was performed. The corresponding intensity feature points between the previous time point (tₙ₋₁ or tₙ₋₂) image, so-called reference, and each further image, so-called floating image, were automatically identified using the key-point detector SURF and used for aligning imaging date with respect to the reference data. Intensity features present at the reference image were classified as background while intensity features appearing on further images (cells/bugs) were classified as foreground. The position of given individual microcolonies have been marked in consecutive images. Setting the background allows enhanced detection of microcolonies and their growth.

### Example14: Performing Broth Microdilution Using Commercial Plates

1. 3-5 colonies were added to water and a 0.5 McFarland cell suspension was prepared.
2. Depending on the target cell, 1 μL, 10 μL, or 30 μL of the cell suspension was added into Mueller Hinton Broth (MHB) growth media to reach a volume of 50 μL in each well of a microwell plate.
4. The plate was sealed and incubated at 34-36 C in a non-CO2 incubator for 18-24 hours.
5. The wells were inspected for turbidity.

### Example15: Performing LD-AST

1. Microbial stock suspension is prepared according to the method of example 1.
2. The stock is diluted by adding TSB media to a concentration of 10⁵ CFU/mL.
3. The LD-AST unit is coupled with the corresponding gel in microwell strip.
4. 1 μL of the sample from step 2 is dispended into the ROI of each LD-AST unit.
5. The strip is incubated at 37° C. for three hours.
6. The ROIs are imaged.
7. The strip is incubated for 1 more hour.
8. The ROIs are imaged.
9. The two images are compared according to the method of example 13.
10. The strips is incubated overnight for verifying the presence of growth.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1-109. (canceled)

110. A method of determining an effect of a chemical agent on growth of microbial cells, the method comprising:
providing a microbial cell suspension containing the microbial cells;
providing a solid support that at least partially surrounds an aperture, the solid support comprising a contact surface, wherein a chemical agent is provided on the contact surface and/or impregnated beneath the contact surface;
contacting a solid phase growth medium with the contact surface of the solid support such that a subregion of the solid phase growth medium is accessible through the aperture, and such that at least a portion of the chemical agent diffuses inwardly into the subregion;
depositing a volume of the microbial cell suspension onto a surface of the subregion, such that microbial cells within the microbial cell suspension are retained on the surface of the subregion;
incubating the solid phase growth medium over a time duration that is sufficiently long to permit exposure of the retained microbial cells to the chemical agent; and
detecting a presence or absence of microbial cell growth within the subregion.

111. The method according to claim 110 wherein the contact surface comprises a planar contact surface, and wherein the solid support is contacted with the solid phase growth medium such that the planar contact surface contacts a surface of the solid phase growth medium and at least partially surrounds the subregion, and such that a portion of the chemical agent diffuses from the planar contact surface into the subregion.

112. The method according to claim 111 wherein the solid support fully surrounds the aperture.

113. The method according to claim 112 wherein the solid support further comprises a flashing feature residing adjacent to the aperture, the flashing feature being configured such that when the planar contact surface is contacted with the solid phase growth medium, the flashing feature is submerged beneath the surface of the solid phase growth medium, thereby preventing or reducing ingress of the microbial cell suspension between the contact surface and the surface of the solid phase growth medium.

114-116. (canceled)

117. The method according to claim 111 wherein the solid support comprises a lateral confinement component located further from the aperture than the planar contact surface, the lateral confinement component being configured such that when the planar contact surface is contacted with the solid phase growth medium, the lateral confinement component is submerged within the solid phase growth medium.

118. (canceled)

119. The method according to claim 111 wherein the contact surface comprises a lateral contact surface located further from the aperture than the planar contact surface, the lateral contact surface being configured such that when the planar contact surface is contacted with the solid phase growth medium, the lateral contact surface is submerged within the solid phase growth medium with the lateral contact surface facing toward the subregion, such that chemical agent diffuses from both the planar contact surface and the lateral contact surface into the subregion.

120. (canceled)

121. The method according to claim 119 wherein the lateral contact surface is configured such that when the planar contact surface is contacted with the solid phase growth medium, the lateral contact surface is inserted into the solid phase growth medium to a depth exceeding 1 mm.

122. (canceled)

123. The method according to claim 110 wherein the solid support comprises a tubular component, and wherein at least a distal surface region of an inner surface of the tubular component is coated with and/or impregnated with the chemical agent, and wherein the tubular component is contacted with the solid phase growth medium such that at least a portion of the distal surface region is submerged within the solid phase growth medium, and such that the chemical agent diffuses inwardly within the subregion of the solid phase growth medium that resides within a lumen of the tubular component.

124. The method according to claim 123 wherein the tubular component is inserted into the solid phase growth medium such that a proximal portion of the tubular component extends outwardly from the solid phase growth medium, and wherein the volume of the microbial cell suspension is dispensed into the proximal portion of the tubular component.

125. The method according to claim 123 wherein the tubular component is inserted such that a distal end of the tubular component contacts a support surface that supports the solid phase growth medium, thereby enclosing the subregion and confining diffusion of the chemical agent within the tubular component.

126. The method according to claim 125 wherein the support surface comprises one or more mating features provided therein or thereon, the one or more mating features being configured to contact the distal end of the tubular component.

127-132. (canceled)

133. The method according to claim 110 wherein one or more of an area density and a subsurface density of the chemical agent spatially varies along the contact surface.

134-135. (canceled)

136. The method according to claim 110 wherein the chemical agent is provided on the contact surface with a suitable quantity and a suitable spatial distribution such that a concentration of the chemical agent immediately below a central portion of the surface of the subregion varies by less than 10% between one hour and three hours after contacting the contact surface with the solid phase growth medium.

137-139. (canceled)

140. The method according to claim 110 wherein the solid phase growth medium is contacted with the contact surface such that a concentration of the chemical agent immediately below a central portion of the surface of the subregion reaches a maximum concentration within 30 minutes of contact between the solid phase growth medium and the contact surface.

141. The method according to claim 110 wherein the solid support comprises a hydrophobic upper surface configured to facilitate retention of the volume of the microbial cell suspension over the subregion.

142. The method according to claim 141 wherein the hydrophobic upper surface is beveled toward the aperture to assist in retention of the volume of the microbial cell suspension over the subregion.

143. The method according to claim 110 wherein a minimum width of the aperture is less than 5 mm.

144-145. (canceled)

146. The method according to claim 110 wherein the number of microbial cells within the volume of the microbial cell suspension deposited onto the surface of the subregion is less than 50.

147-148. (canceled)

149. The method according to claim 110 wherein the volume of the microbial cell suspension deposited onto the surface of the subregion is less than 5 microliters.

150-155. (canceled)

156. The method according to claim 110 wherein the microbial cell suspension is obtained by processing a whole blood sample in an absence of blood culture.

157. The method according to claim 110 wherein the microbial cell suspension is obtained from a blood culture bottle in an absence of performing subculture.

158-159. (canceled)

160. The method according to claim 110 further comprising providing one or more additional solid supports, each additional solid support at least partially surrounding a respective additional aperture, each additional solid support comprising a respective additional contact surface, wherein each additional contact surface has a different amount of the chemical agent provided thereon and/or impregnated therebeneath;
contacting the solid phase growth medium with each additional contact surface such that additional subregions of the solid phase growth medium are accessible through the respective additional apertures, and such that at least a portion of the chemical agent diffuses inwardly into each respective additional subregions from each respective additional contact surface;
depositing additional volumes of the microbial cell suspension onto a respective surface of each additional subregion, such that microbial cells within the microbial cell suspension are retained on the respective surfaces of the additional subregions; and
after incubating the solid phase growth medium, detecting a presence or absence of microbial cell growth within each subregion.

161. The method according to claim 160 further comprising determining a minimum inhibitory concentration of the chemical agent based on the assessment of the presence or absence of microbial cell growth within the subregions.

162. The method according to claim 161 wherein the minimum inhibitory concentration is determined according to an estimated concentration or concentration range of the chemical agent below the surface of each subregion during incubation of the solid phase growth medium.

163. The method according to claim 160 wherein the solid support and the additional solid supports are mechanically coupled and form an array of solid supports.

164. The method according to claim 163 wherein the solid phase growth medium is supported by a solid phase growth medium support structure, the support structure comprising a plurality of microwells, each microwell comprising a respective volume of the solid phase growth medium, and wherein the array of solid supports is contacted with the solid phase growth medium such that each contact surface of the array of solid supports is contacted with a different respective volume of the solid phase growth medium in a different respective microwell.

165. The method according to claim 164 wherein one or more of the array of solid supports and the solid phase growth medium support structure comprise a keyed feature that facilitates alignment between the respective contact surfaces and the respective microwells.

166. (canceled)

167. A method of determining an effect of a chemical agent on growth of microbial cells, the method comprising:
providing a microbial cell suspension containing the microbial cells;
contacting a solid phase growth medium with the chemical agent at one or more contact regions that at least partially surround and reside adjacent to a subregion of the solid phase growth medium, such that at least a portion of the chemical agent diffuses into the subregion from the one or more contact regions, wherein the one or more contact regions are provided such that a spatial extent of the subregion, when measured in at least one direction parallel to a surface of the solid phase growth medium, is less than 5 mm;
depositing a volume of the microbial cell suspension onto a surface of the subregion, such that microbial cells within the microbial cell suspension are retained on the surface of the subregion;
incubating the solid phase growth medium over a time duration that is sufficiently long to permit exposure of the retained microbial cells to the chemical agent; and
detecting a presence or absence of microbial cell growth within the subregion.

168. A method of introducing a chemical agent into a solid phase growth medium, the method comprising:
providing a solid support that at least partially surrounds an aperture, the solid support comprising a contact surface, wherein a chemical agent is provided on the contact surface and/or impregnated beneath the contact surface;
contacting the solid phase growth medium with the contact surface of the solid support such that a subregion of the solid phase growth medium is accessible through the aperture, and such that at least a portion of the chemical agent diffuses inwardly into the subregion.

169-202. (canceled)
